(19) United States
(12) Patent Application Publication
(10) Pub. No.: US 2025/0163076 A1
(43) Pub. Date: May 22, 2025

(54) SYNTHESIS OF BRUTON'S TYROSINE KINASE INHIBITORS

(71) Applicant: JANSSEN PHARMACEUTICA NV, Beerse (BE)

(72) Inventors: Ngoc Duc TRAN, Turnhout (BE); Christos XIOURAS, Antwerpen (BE); Edward CLEATOR, Cambridgeshire (GB); William Marc MATON, Mol (BE); Thomas Joachim Landewald RAMMELOO, Vosselaar (BE); Philippe FERNANDES, Turnhout (BE); Jean-Pierre A BONGARTZ, Turnhout (BE); Duy Chi Trung CAO, Zurich (CH); Matthew Allan HORWITZ, Murcia (ES); Xavier Jean-Marie JUSSEAU, La Madeleine (FR); Sébastien Francois-Emmanuel LEMAIRE, Schaerbeek (BE); Bertrand SCHWEITZER-CHAPUT, Antwerpen (BE)

(21) Appl. No.: 18/838,678

(22) PCT Filed: Feb. 17, 2023
(86) PCT No.: PCT/EP2023/054043

§ 371 Date: Aug. 15, 2024

(30) Foreign Application Priority Data
Feb. 18, 2022 (GR) 20220100151
Apr. 6, 2022 (EP) 22167032.6
Jun. 10, 2022 (EP) 22178413.5

(51) Int. Cl. C07D 495/16

(52) U.S. Cl. CPC C07D 495/16

(57) ABSTRACT

The present disclosure is directed to methods of synthesizing a compound of Formula (I), where the R groups are defined herein, and to methods of synthesizing a compound of Formula (II). Some aspects of the present disclosure is directed to converting the compound of Formula (II) into its P form.

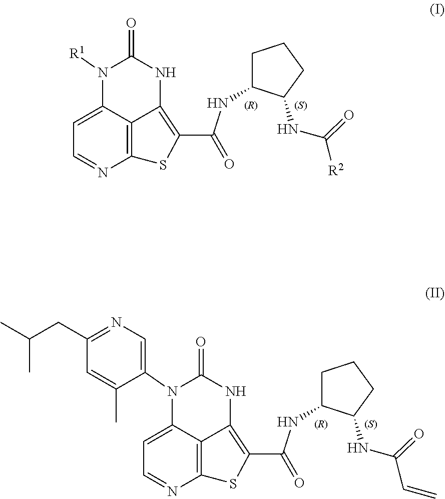

### CLAIM OF PRIORITY

This application claims priority to Greek Application No. GR20220100151 filed on Feb. 18, 2022, European Application No. EP 22167032.6 filed on Apr. 6, 2022, and European Application No. EP22178413.5 filed on Jun. 10, 2022, each of which is incorporated by reference in its entirety.

### TECHNICAL FIELD

The present disclosure is directed to the synthesis of small molecule tyrosine kinase inhibitors.

### BACKGROUND

Human Bruton's tyrosine kinase (“BTK”) is a ˜76 kDa protein belonging to the Tec family of non-receptor tyrosine kinases. Tec kinases form the second largest family of cytoplasmic tyrosine kinases in mammalian cells, which consists of four other members in addition to BTK: the eponymous kinase TEC, ITK, TXK/RLK and BMX. Tec kinases are evolutionarily conserved throughout vertebrates. They are related to, but structurally distinct from, the larger Src and Syk kinase families. Tec family proteins are abundantly expressed in hematopoietic tissues and play important roles in the growth and differentiation of blood and endothelial cells in mammals. Based upon BTK expression from IHC studies described in the art, Btk inhibition has the potential to modulate biology associated with B cells, macrophages, mast cells, osteoclasts, and platelet microparticles. Corneth, O. B., et al. Curr. Top. Microbiol. Immunol. BTK Signaling in B Cell Differentiation and Autoimmunity. 2015 Sep. 5.

### SUMMARY

Disclosed herein are methods of synthesizing a compound of Formula (I), including a solvate or a pharmaceutically acceptable salt form thereof, where the R groups are defined herein:

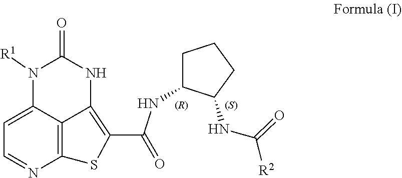

Also disclosed are methods of synthesizing a compound of Formula (II), including a solvate or a pharmaceutically acceptable salt form thereof:

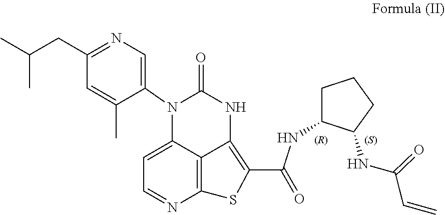

The compound of Formula (II) is also known as N-((1R,2S)-2-acrylamidocyclopentyl)-5-(6-isobutyl-4-methylpyridin-3-yl)-4-oxo-4,5-dihydro-3H-1-thia-3,5,8-triazaacenaphthylene-2-carboxamide. In some embodiments, the compound of Formula (II) exists as a mixture of atropisomers-P and M forms.

Some aspects further comprise converting the compound of Formula (II) into its P form:

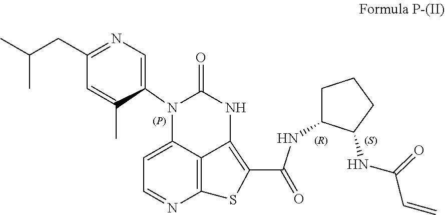

The compound of Formula P-(II) is also known as N-((1R,2S)-2-acrylamidocyclopentyl)-5-(S)-(6-isobutyl-4-methylpyridin-3-yl)-4-oxo-4,5-dihydro-3H-1-thia-3,5,8-triazaacenaphthylene-2-carboxamide.

The compound of Formula M-(II) is also known as N-((1R,2S)-2-acrylamidocyclopentyl)-5-(R)-(6-isobutyl-4-methylpyridin-3-yl)-4-oxo-4,5-dihydro-3H-1-thia-3,5,8-triazaacenaphthylene-2-carboxamide.

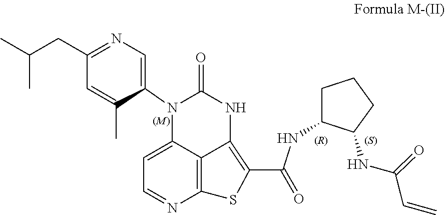

### FIGURES

FIG. 1 depicts examples of reactor configuration used for the CIDT process.

FIG. 2 depicts typical evolution of P (left bar in each time point) and M (right bar in each time point) isomer concentrations in the liquid during re-circulation in the loop reactor in 1-propanol/water system.

FIG. 3 depicts typical evolution of P (left bar in each time point) and M (right bar in each time point) isomer concentrations in the liquid during re-circulation in the loop reactor in propylene carbonate/water system.

FIG. 4 depicts X-ray powder diffraction (XRPD) the spectrum of crystalline Form B of Formula P-(II) dihydrate.

FIG. 5 depicts the infrared (IR) spectrum of crystalline Form B of Formula P-(II) dihydrate.

FIG. 6 depicts the differential scanning calorimetry (DSC) spectrum of crystalline Form B of Formula P-(II) dihydrate utilizing standard pan conditions.

FIG. 7 depicts the DSC spectrum of crystalline Form B of Formula P-(II) dihydrate utilizing Tzero hermetically sealed pan conditions.

FIG. 8 depicts the thermogravimetry (TGA) spectrum of crystalline Form B of Formula P-(II) dihydrate utilizing Tzero hermetically sealed pan conditions.

FIG. 9 depicts the decay of the diastereomeric excess in propylene carbonate at 100° C. measured by HPLC (left), and Linear plot of t vs ln (1/d.e.), the slope of which gives the value of 2kₑ (right).

### DETAILED DESCRIPTION

The disclosure may be more fully appreciated by reference to the following description, including the following glossary of terms and the concluding examples. It is to be appreciated that certain features of the disclosed compositions and methods which are, for clarity, described herein in the context of separate aspects, may also be provided in combination in a single aspect. Conversely, various features of the disclosed compositions and methods that are, for brevity, described in the context of a single aspect, may also be provided separately or in any subcombination. It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to aspects containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

The term “about” as used herein when immediately preceding a numerical value means a range of plus or minus 10% of that value, for example, “about 50” means 45 to 55, “about 25,000” means 22,500 to 27,500, etc., unless the context of the disclosure indicates otherwise, or is inconsistent with such an interpretation.

The term “alkyl,” when used alone or as part of a substituent group, refers to a straight- or branched-chain alkyl group having from 1 to 12 carbon atoms (“C₁₋₁₂”), preferably 1 to 6 carbons atoms (“C₁₋₆”), in the chain. Examples of alkyl groups include methyl (Me, C₁alkyl) ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and groups that in light of the ordinary skill in the art and the teachings provided herein would be considered equivalent to any one of the foregoing examples.

When a range of carbon atoms is used herein, for example, C₁-6, all ranges, as well as individual numbers of carbon atoms are encompassed. For example, “C₁₋₃” includes C₁₋₃, C₁₋₂, C₂₋₃, C₁, C₂, and C₃.

The term “C₁₋₆alk” refers to an aliphatic linker having 1, 2, 3, 4, 5, or 6 carbon atoms and includes, for example, CH₂, CH(CH₃), CH(CH₃)—CH₂, and C(CH₃)₂—. The term “—C₀alk-” refers to a bond. In some aspects, the C₁₋₆alk can be substituted with an oxo group or an OH group.

The term “alkenyl,” when used alone or as part of a substituent group, refers to straight and branched carbon chains having from 2 to 12 carbon atoms (“C₂₋₁₂”), preferably 2 to 6 carbon atoms (“C₂₋₆”), wherein the carbon chain contains at least one, preferably one to two, more preferably one double bond. For example, alkenyl moieties include, but are not limited to allyl, 1-propen-3-yl, 1-buten-4-yl, propa-1,2-dien-3-yl, and the like.

The term “alkynyl,” when used alone or as part of a substituent group, refers to straight and branched carbon chains having from 2 to 12 carbon atoms (“C₂₋₁₂”), preferably 2 to 6 carbon atoms (“C₂₋₆”), wherein the carbon chain contains at least one, preferably one to two, more preferably one triple bond. For example, alkynyl moieties include, but are not limited to vinyl, 1-propyn-3-yl, 2-butyn-4-yl, and the like.

The term “aryl” refers to carbocylic aromatic groups having from 6 to 10 carbon atoms (“C₆₋₁₀”) such as phenyl, naphthyl, and the like.

The term “cycloalkyl” refers to monocyclic, non-aromatic hydrocarbon groups having from 3 to 10 carbon atoms (“C₃₋₁₀”), preferably from 3 to 6 carbon atoms (“C₃₋₆”). Examples of cycloalkyl groups include, for example, cyclopropyl (C₃), cyclobutyl (C₄), cyclopentyl (C₅), cyclohexyl (C₆), 1-methylcyclopropyl (C₄), 2-methylcyclopentyl (C₄), adamantanyl (C₁₀) and the like.

The term “heterocycloalkyl” refers to any five to ten membered monocyclic or bicyclic, saturated ring structure containing at least one heteroatom selected from the group consisting of O, N and S. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahyofuranyl, tetrahydropyranyl, piperazinyl, hexahydro-5H-[1,4]dioxino[2,3-c]pyrrolyl, benzo[d][1,3]dioxolyl, and the like.

The term “heteroaryl” refers to a mono- or bicyclic aromoatic ring structure including carbon atoms as well as up to four heteroatoms selected from nitrogen, oxygen, and sulfur. Heteroaryl rings can include a total of 5, 6, 9, or 10 ring atoms (“C₅₋₁₀”). Examples of heteroaryl groups include but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like.

The term “halogen” represents chlorine, fluorine, bromine, or iodine. The term “halo” represents chloro, fluoro, bromo, or iodo.

The term “haloalkyl” refers to an alkyl moiety wherein one or more of the hydrogen atoms has been replaced with one or more halogen atoms. One exemplary substitutent is fluoro. Preferred haloalkyl groups of the disclosure include trihalogenated alkyl groups such as trifluoromethyl groups.

The term “oxo” refers to a ═O moiety, wherein two hydrogens from the same carbon atom have be replaced with a carbonyl. For example, an oxo-substituted pyrrolidinyl moiety could be a pyrrolidin-2-one moiety or a pyrrolidin-3-one moiety.

The term “phenyl” represents the following moiety:

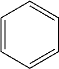

The phenyl moiety can be attached through any of the carbon atoms.

The term “pyridyl” represents the following moiety:

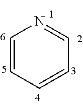

The pyridyl moiety can be attached through any one of the 2-, 3-, 4-, 5-, or 6-position carbon atoms.

“Compounds of the present disclosure,” and equivalent expressions, are meant to embrace compounds of the Formula (I), compounds of the Formula (II), compounds of the Formula P-(II), and compounds of the Formula M-(II), as described herein, which expression includes the pharmaceutically acceptable salts, and the solvates, e.g., hydrates, dihydrates, and polymorphs thereof, where the context so permits. Compounds of the present disclosure also include the stereoisomers (including but not limited to enantiomers and diastereomers) thereof and the tautomeric forms thereof. Similarly, reference to intermediates, whether or not they themselves are claimed, is meant to embrace their salts, and solvates, where the context so permits.

Suitable pharmaceutically acceptable salts of the compounds of disclosure include acid addition salts that can, for example, be formed by mixing a solution of the compound with a solution of a pharmaceutically acceptable acid such as, hydrochloric acid, sulfuric acid, fumaric acid, maleic acid, succinic acid, acetic acid, benzoic acid, citric acid, tartaric acid, carbonic acid or phosphoric acid. Furthermore, where compounds of the disclosure carry an acidic moiety, suitable pharmaceutically acceptable salts thereof may include alkali metal salts such as, sodium or potassium salts; alkaline earth metal salts such as, calcium or magnesium salts; and salts formed with suitable organic ligands such as, quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, and valerate.

As used herein, “solvate” may be a solvate with water (i.e., a hydrate, a dihydrate) or with an organic solvent.

For the purposes of this disclosure, the terms “crystalline form” and “polymorph” are synonymous. Characterizing information for crystalline forms is provided herein. It should be understood that the determination of a particular form can be achieved using any portion of the characterizing information that one skilled in the art would recognize as sufficient for establishing the presence of a particular form. For example, even a single distinguishing peak can be sufficient for one skilled in the art to appreciate that a particular form is present.

As used herein, the term “isotopic variant” refers to a compound that contains unnatural proportions of isotopes at one or more of the atoms that constitute such compound. For example, an “isotopic variant” of a compound can be radiolabeled, that is, contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the disclosure may include the preparation of isotopic variants with radioisotopes, in the instance for example, where the resulting compounds may be used for drug and/or substrate tissue distribution studies. Radiolabeled compounds of the disclosure can be used in diagnostic methods such as Single-photon emission computed tomography (SPECT). The radioactive isotopes tritium, i.e. ³H, and carbon-14, i.e. ¹⁴C, are particularly useful for their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds of the disclosure, radioactive or not, are intended to be encompassed within the scope of the disclosure.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers.

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

“Atropisomers” refer to stereoisomers that arise because of hindered rotation around a single bond. Atropisomers are named as either M or P throughout.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci-and nitro-forms of phenyl nitromethane, that are likewise formed by treatment with acid or base.

Tautomeric forms may be relevant to the attainment of the optimal chemical reactivity and biological activity of a compound of interest.

The compounds of this disclosure may possess one or more asymmetric centers; such compounds can therefore be produced as individual (R)-or(S)-stereoisomers or as mixtures thereof.

Unless indicated otherwise, the description or naming of a particular compound in the specification and claims is intended to include both individual enantiomers and mixtures, racemic or otherwise, thereof. Within the present disclosure, any open valency appearing on a carbon, oxygen, or nitrogen atom in any structure described herein indicates the presence of a hydrogen atom. Where a chiral center exists in a structure, but no specific stereochemistry is shown for that center, both enantiomers, separately or as a mixture, are encompassed by that structure. The methods for the determination of stereochemistry and the separation of stereoisomers are well-known in the art.

### Methods of Synthesis

The present disclosure is directed to a method of synthesizing a compound of Formula (I), including a solvate or a pharmaceutically acceptable salt form thereof:

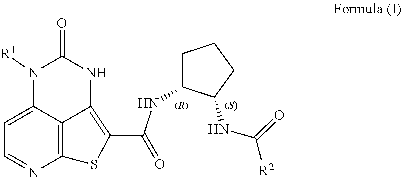

wherein:
   R¹ is selected from C₅₋₁₀ aryl and C₄₋₉ heteroaryl wherein the aryl or heteroaryl are optionally substituted with one or more groups independently selected from C₁₋₆ alkyl; and
   R² is selected from C₁₋₆ alkyl and C₂₋₆ alkenyl;
      comprising contacting the compound

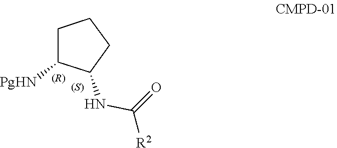

with an acid and a first solvent followed by contacting with the compound

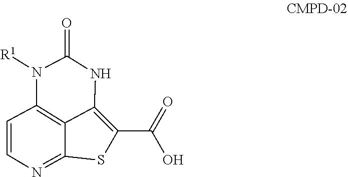

in the presence of one or more coupling reagents, an amine base, and a second solvent to obtain the compound of Formula (I).

The synthesis of a compound of Formula (I) is outlined in Scheme 1

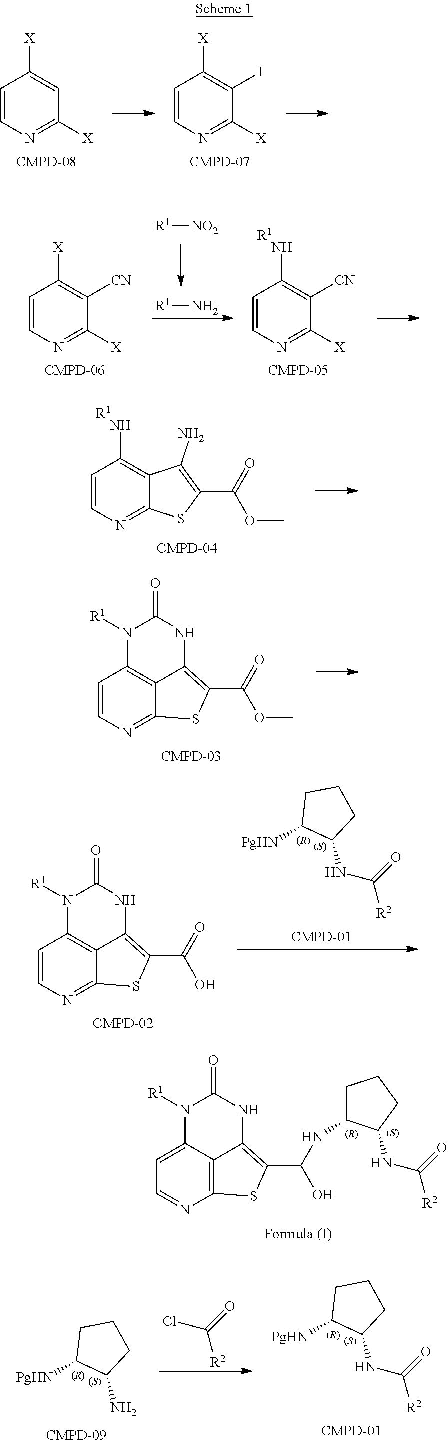

In some aspects R¹ is C₄₋₉ heteroaryl. In some aspects the C₄₋₉ heteroaryl is

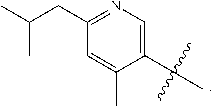 [SMILES: *c1cnc(CC(C)C)cc1C]

In some aspects R² is C₂₋₆ alkenyl. In some aspects the C₂₋₆ alkenyl is

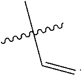 [SMILES: *C=C]

In some aspects the Pg is Boc (tert-butyloxycarbonyl).

In some aspects the acid is methanesulphonic acid. In some aspects the first solvent is selected from dichloromethane (DCM), ethyl acetate (EtOAc), 2-methyltetrahydrofuran (MeTHF), tetrahydrofuran (THF), and acetonitrile (ACN). In some aspects the coupling reagent is selected from 1-Ethyl-3-(3-dimethylaminopropyl)carbodiimide (EDCI), hydroxybenzotriazole (HOBt), 1-[Bis(dimethylamino)methylene]-1H-1,2,3-triazolo[4,5-b]pyridinium 3-oxide hexafluorophosphate (HATU), propylphosphonic anhydride (T3P), 1,1′-carbonyldiimidazole (CDI), and 2-hydroxypyridine-N-oxide (HOPO). In some aspects the amine base is selected from N,N-diisopropylethylamine (DIPEA), triethylamine, tributylamine, N-methyl morpholine, and N-methyl piperidine. In some aspects the second solvent is selected from dichloromethane, ethyl acetate, 2-methyltetrahydrofuran, tetrahydrofuran, and acetonitrile.

In another aspect of the method of synthesizing a compound of Formula (I), the method further comprises contacting the compound

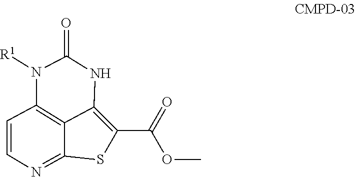

with a base and an ammonium salt in a solvent to form the compound CMPD-02. In some aspects the base is selected from potassium trimethylsilanolate (TMSOK), lithium hydroxide (LiOH), or sodium hydroxide (NaOH). In some aspects the ammonium salt is selected from tetrabutylammonium bromide (TBAB), tetrabutylammonium hydrogen sulfate, benzyltrimethylammonium bromide, benzyltrimethylammonium chloride, and Aliquat 336. In some aspects the solvent is tetrahydrofuran.

In another aspect of the method of synthesizing a compound of Formula (I), the method further comprises contacting the compound

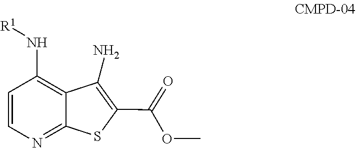

with a carbonyl source and a solvent to form the compound CMPD-03. In some aspects the carbonyl source is selected from N,N′-carbonyldiimidazole (CDI), phosgene, triphosgene, and N,N′-Disuccinimidyl carbonate (DSC). In some aspects the solvent is tetrahydrofuran.

In another aspect of the method of synthesizing a compound of Formula (I), the method further comprises contacting the compound

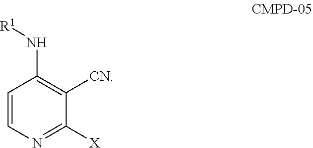

with a sulfur reagent and a base in a solvent to form the compound CMPD-04 and wherein X is selected from Cl and Br. In some aspects X is Cl. In some aspects the sulfur reagent methyl 2-mercaptoacetate. In some aspects the base is sodium methoxide (NaOMe). In some aspects the solvent is methanol.

In another aspect of the method of synthesizing a compound of Formula (I), the method further comprises contacting the compound CMPD-06

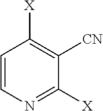

with the compound R¹—NH₂ with a base, and an amine base in a solvent system to form the compound CMPD-05. In some aspects X is Cl. In some aspects R¹ is

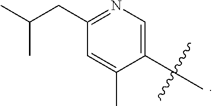 [SMILES: *c1cnc(CC(C)C)cc1C]

In some aspects the base is K₂CO₃. In some aspects the amine base is N,N-diisopropylethylamine (DIPEA). In some aspects the solvent system is toluene/NMP.

In another aspect of the method of synthesizing a compound of Formula (I), the method further comprises contacting the compound R¹—NO₂ with a hydrogen source, a catalyst, and a solvent system to form the compound R¹—NH₂. In some aspects the hydrogen source is ammonium formate, NaBH₄ or H₂. In some aspects the catalyst is from NiCl₂, Pd/C, palladium II acetate (Pd(OAc)₂), Pd(OH)₂. Raney Ni, Sponge Ni, Pt/V/C, Pt/Fe/C, NiBr₂, NiCl₂. In some aspects the solvent system is selected from toluene/water, xylene, THF, selected MeTHF, MeOH, THF/MeOH, Toluene/MeOH, MeTHF/water, and THF/water.

In another aspect of the method of synthesizing a compound of Formula (I), the method further comprises contacting the compound CMPD-07

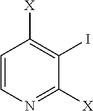

with a cyano source and a solvent to form the compound CMPD-06. In some aspects X is Cl. In some aspects the cyano source is CuCN. In some aspects the solvent is butyronitrile.

In another aspect of the method of synthesizing a compound of Formula (I), the method further comprises contacting the compound

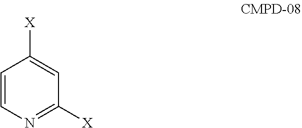

with a base and an electrophilic halogen in a solvent to form the compound CMPD-07. In some aspects X is Cl. In some aspects the base is lithium diisopropylamine. In some aspects the solvent is tetrahydrofuran.

In another aspect of the method of synthesizing a compound of Formula (I), the

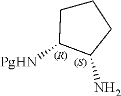

method further comprises contacting the compound CMPD-09 with a base,

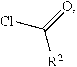

and a solvent to form the compound CMPD-01. In some aspects R² is C₂₋₆ alkenyl. In some aspects the C₂₋₆ alkenyl is

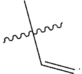 [SMILES: *C=C]

In some aspects the Pg is Boc. In some aspects the base is NaHCO₃. In some aspects the solvent is 2-methyltetrahydrofuran.

In another aspect of the method of synthesizing a compound of Formula (I), the the method further comprises synthesizing CMPD-04 according to Scheme 2:

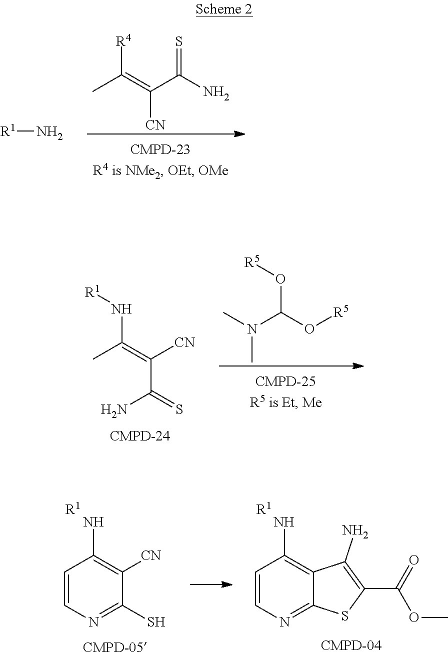

In another aspect of the method of synthesizing a compound of Formula (I), the method further comprises contacting the compound

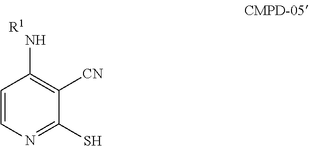

with an alkylating reagent in the presence of a base in a solvent to form the compound CMPD-04. In some aspect the alkylating reagent is methyl chloroacetate. In some aspects the base is selected from sodium carbonate (Na₂CO₃) and potassium carbonate (K₂CO₃). In some aspects the solvent is selected from one or more solvents such as N,N-dimethylformamide (DMF), acetonitrile (ACN), methanol, 2-methyltetrahydrofuran (MeTHF), and water.

In another aspect of the method of synthesizing a compound of Formula (I), the method further comprises contacting the compound

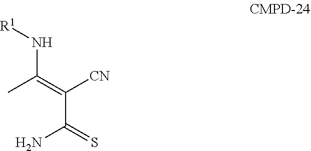

with the compound

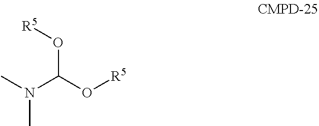

and a base in a solvent to form the compound CMPD-05′ wherein R⁵ is C₁₋₆alkyl. In some aspects C₁₋₆alkyl is methyl and ethyl. In some aspects CMPD-25 is selected from N,N-dimethylformamide dimethyl acetal and N,N-dimethylformamide diethyl acetal. In some aspects the base is selected from sodium hydroxide (NaOH), potassium phosphate tribasic (K₃PO₄), potassium carbonate (K₂CO₃), 1,8-diazabicyclo[5.4.0]undec-7-ene (DBU) and morpholine. In some aspects the solvent is selected from one or more solvents such as N,N-dimethylformamide (DMF), isopropyl acetate, 2-methyltetrahydrofuran (MeTHF), tetrahydrofuran (THF), acetonitrile (ACN). toluene, ethanol, tert-butyl methyl ether (TBME) and water.

In another aspect of the method of synthesizing a compound of Formula (I), the method further comprises contacting the compound

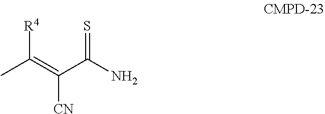

with compound R¹—NH₂ in a solvent to form the compound CMPD-24, wherein R⁴ is (C₁₋₆alkyl)₂N and C₁₋₆alkylO. In some aspects C₁₋₆alkyl is selected from methyl and ethyl. In some aspects the solvent is selected from one or more solvents such as N,N-dimethylformamide (DMF), 1-methyl-2-pyrrolidone (NMP), isopropyl acetate, 2-methyltetrahydrofuran (MeTHF), tetrahydrofuran (THF), acetonitrile (ACN), ethanol, and methanol.

In another aspect of the method of synthesizing a compound of Formula (I), the method further comprises synthesis of CMPD-02 according to scheme 3.

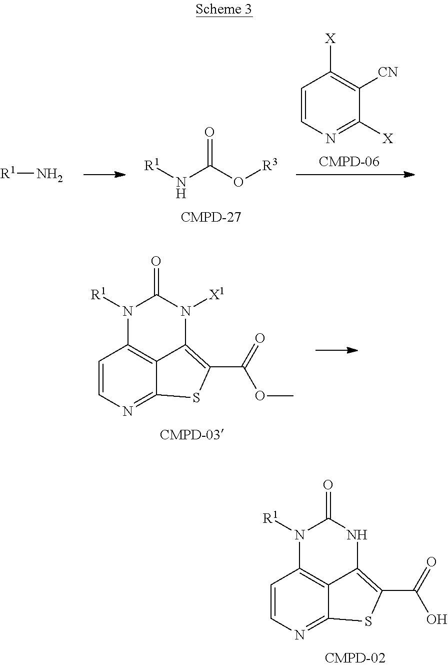

In another aspect of the method of synthesizing a compound of Formula (I), the method further comprises contacting the compound

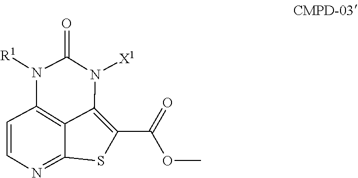

where X¹ is H or Na with a base and an ammonium salt in a solvent to form the compound CMPD-02. In some aspects the base is selected from potassium trimethylsilanolate (TMSOK), lithium hydroxide (LiOH), potassium hydroxide, (KOH), or sodium hydroxide (NaOH). In some aspects the ammonium salt is selected from tetramethyl ammonium bromide, tetraethylammonium bromide, tetraethyl ammonium chloride, tetrabutylammonium bromide (TBAB), tetrabutylammonium hydrogen sulfate, benzyltrimethylammonium bromide, benzyltrimethylammonium chloride, didodecyldimethylammonium bromide and Aliquat 336. More generally, those skilled in the art will recognize that alkylammonium salts and substituted alkylammonium salts may be used. In some aspects the solvent is tetrahydrofuran or methyltetrahydrofuran.

In another aspect of the method of synthesizing a compound of Formula (I), the method further comprises contacting the compound CMPD-06

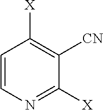

wherein X is selected from Cl and Br, with the compound

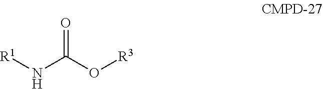

where R³ is C₁₋₆ alkyl and with a sulfur reagent, in the presence of one or more base and an ammonium salt in a solvent to form the compound CMPD-03′, where X¹ is H or Na. In some aspects, the compond CMPD-03′ where X¹ is H may be converted to CMPD-03′ where X¹ is Na. More generally, those skilled in the art will recognize that X¹ may be a monovalent alkali metal or an ammonium. In some aspects X is Cl. In some aspects C₁₋₆ alkyl is selected from methyl, ethyl and tert-butyl. In some aspects the sulfur reagent is methyl 2-mercaptoacetate In some aspects the base is selected from one or more bases such as sodium hydroxide (NaOH), potassium hydroxide (KOH), potassium phosphate tribasic (K₃PO₄), sodium carbonate (Na₂CO₃), potassium carbonate (K₂CO₃), 1,8-diazabicyclo[5.4.0]undec-7-ene (DBU), 1,1,3,3-tetramethylguanidine (TMG), sodium methanolate (NaOMe), sodium tert-butoxide (t-BuONa). In some aspects the ammonium salt is selected from tetrabutylammonium bromide (TBAB), tetrabutylammonium chloride (TBAC), benzyltrimethylammonium chloride and tetramethylammonium chloride. More generally, those skilled in the art will recognize that alkylammonium salts and substituted alkylammonium salts may be used. In some aspects the solvent is selected from one or more solvents such as N,N-dimethylformamide (DMF), isopropyl acetate, 2-methyltetrahydrofuran (MeTHF), tetrahydrofuran (THF), acetonitrile (ACN). toluene, tetrahydrofuran (THF), methanol, isopropyl alcohol, tert-butyl methyl ether (TBME) and water.

In another aspect of the method of synthesizing a compound of Formula (I), the method further comprises contacting the compound R¹—NH₂ with an acylating agent in the presence or absence of a base or an acid in a solvent to form the compound CMPD-27, wherein R³ is C₁₋₆ alkyl. In some aspects C₁₋₆ alkyl is selected from methyl, ethyl and tert-butyl. In some aspects the acylating agent is selected from methyl chloroformate, dimethyl carbonate, ethyl chloroformate, diethyl carbonate, di-tert-butyl dicarbonate. In some aspects the acid is selected from acetic acid (AcOH), pivalic acid (PivOH), ammonium chloride (NH₄Cl), sodium hydrogen sulfate (NaHSO₄). In some aspects the base is selected from potassium phosphate tribasic (K₃PO₄), potassium carbonate (K₂CO₃), sodium carbonate (Na₂CO₃), cesium carbonate (Cs₂CO₃), sodium hydrogencarbonate (NaHCO₃) and sodium bis(trimethylsilyl)amide (NaHMDS), potassium tert-butoxide. In some aspects the solvent is selected from one or more solvents such as N,N-dimethylformamide (DMF), 1-methyl-2-pyrrolidone (NMP), isopropyl acetate, ethyl acetate (EtOAc), toluene, acetone, 2-methyltetrahydrofuran (MeTHF), tetrahydrofuran (THF), acetonitrile (ACN), isopropyl alcohol, ethanol, and methanol, and water. 4-Methyl-2-pentanone, tert-butyl methyl ether (TBME), cyclopentyl methyl ether (CPME).

In another aspect of the method of synthesizing a compound of Formula (I), the method further comprises synthesis of CMPD-01 according to scheme 4.

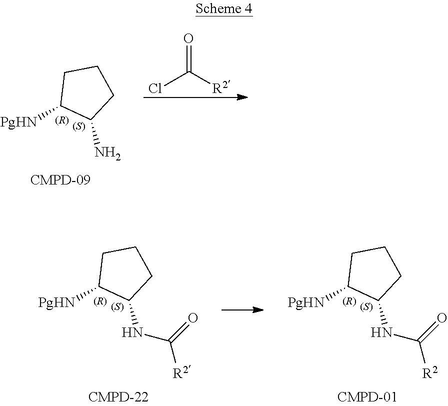

In some aspects, conversion of CMPD-09 to CMPD-01 may be accomplished by reaction with ClC(O)—R²′, in the presence of one or more base and a solvent, wherein R²′ is a precursor to R² C₂₋₆ alkenyl. In some aspects, R²′ is

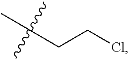 [SMILES: *CCCl]

which may be converted to

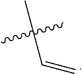 [SMILES: *C=C]

The conversion from CMPD-09 to CMPD-01 may be accomplished with or without isolation of CMPD-22. In some aspects the base is selected from one or more of the following: sodium hydrogen carbonate (NaHCO₃), lithium hydroxide (LiOH), sodium hydroxide (NaOH), potassium hydroxide (KOH), potassium phosphate monobasic (KH₂PO₄), potassium phosphate dibasic (K₂HPO₄), potassium phosphate tribasic (K₃PO₄), sodium carbonate (Na₂CO₃), potassium carbonate (K₂CO₃), pyridine, 1,8-diazabicyclo[5.4.0]undec-7-ene (DBU), 1,1,3,3-tetramethylguanidine (TMG), N,N-diisopropylethylamine (DIPEA), triethylamine (TEA), and N,N-dicyclohexylmethylamine (Cy₂NMe). In some aspects the solvent is selected from one or more solvents such as isopropyl acetate, ethyl acetate (EtOAc), toluene, 2-methyltetrahydrofuran (MeTHF), tetrahydrofuran (THF), acetonitrile (ACN), 4-Methyl-2-pentanone, tert-butyl methyl ether (TBME), cyclopentyl methyl ether (CPME) and water.

The present disclosure is also directed to a method of synthesizing a compound of Formula (II), including a solvate or a pharmaceutically acceptable salt form thereof:

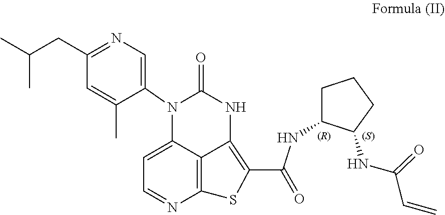

comprising contacting the compound

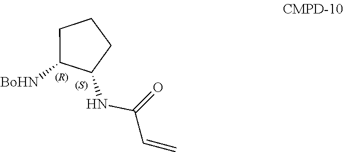

with an acid and a first solvent followed by contacting with the compound

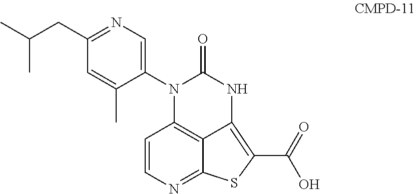 [SMILES: Cc1cc(CC(C)C)ncc1N1C(=O)Nc2c(C(=O)O)sc3nccc1c23]

in the presence of one or more coupling reagents, an amine base, and a second solvent to obtain the compound of Formula (II).

In some aspects the acid is methanesulphonic acid. In some aspects the first solvent is selected from dichloromethane (DCM), ethyl acetate (EtOAc), 2-methyltetrahydrofuran (MeTHF), tetrahydrofuran (THF), and acetonitrile (ACN). In some aspects the coupling reagent is selected from 1-Ethyl-3-(3-dimethylaminopropyl) carbodiimide (EDCI), hydroxybenzotriazole (HOBt), 1-[Bis(dimethylamino)methylene]-1H-1,2,3-triazolo[4,5-b]pyridinium 3-oxide hexafluorophosphate (HATU), propylphosphonic anhydride (T3P), 1,1′-carbonyldiimidazole (CDI), and 2-hydroxypyridine-N-oxide (HOPO). In some aspects the amine base is selected from N,N-diisopropylethylamine (DIPEA), triethylamine, tributylamine, N-methyl morpholine, and N-methyl piperidine. In some aspects the second solvent is selected from dichloromethane (DCM), ethyl acetate (EtOAc), 2-methyltetrahydrofuran (MeTHF), tetrahydrofuran (THF), and acetonitrile (ACN).

In some aspects, the synthesis of a compond of Formula (II) is according to scheme 5.

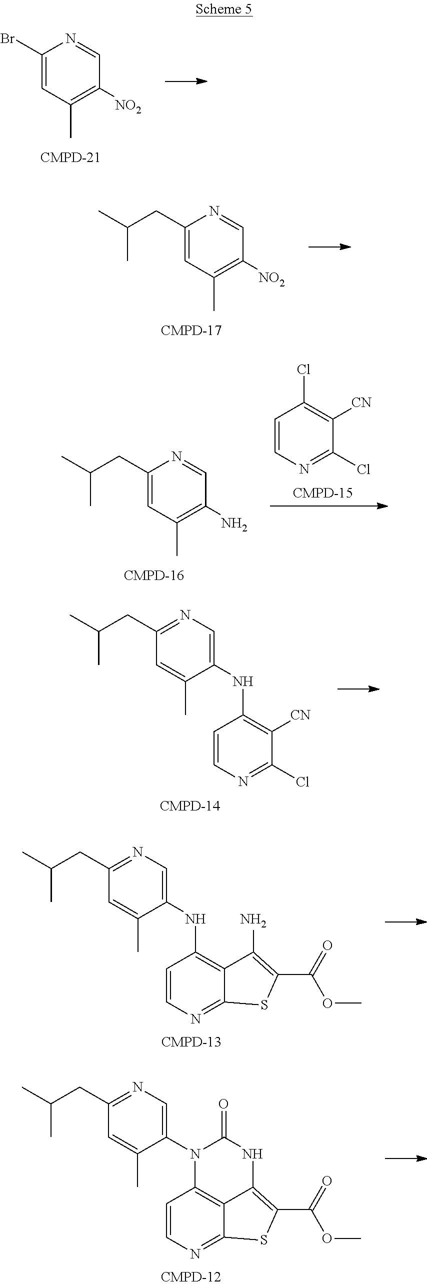

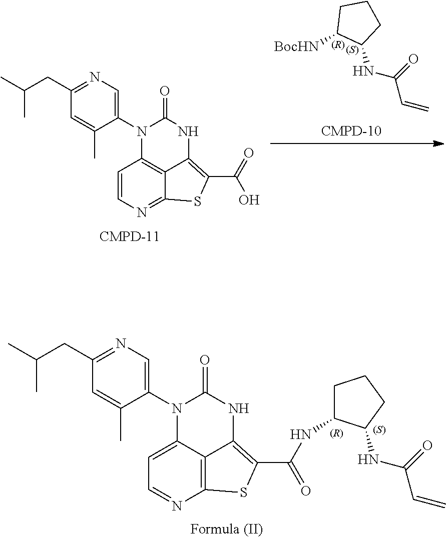

In another aspect of the method of synthesizing a compound of Formula (II), the method further comprises contacting the compound

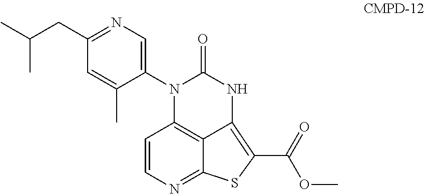 [SMILES: COC(=O)c1sc2nccc3c2c1NC(=O)N3c1cnc(CC(C)C)cc1C]

with a base and an ammonium salt in a solvent to form the compound CMPD-11. In some aspects the base is potassium trimethylsilanolate (TMSOK). In some aspects the ammonium salt is selected from tetrabutylammonium bromide (TBAB), tetrabutylammonium hydrogen sulfate, benzyltrimethylammonium bromide, benzyltrimethylammonium chloride, and Aliquat 336. In some aspects the solvent is tetrahydrofuran.

In another aspect of the method of synthesizing a compound of Formula (II), the method further comprises contacting the compound

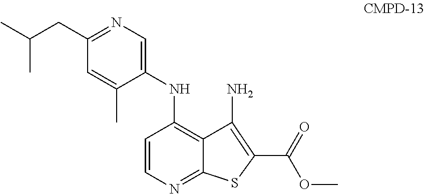 [SMILES: COC(=O)c1sc2nccc(Nc3cnc(CC(C)C)cc3C)c2c1N]

with a carbonyl source and a solvent to form the compound CMPD-12. In some aspects the carbonyl source is selected from N,N′-carbonyldiimidazole (CDI), phosgene, triphosgene, and N,N′-Disuccinimidyl carbonate (DSC). In some aspects the solvent is tetrahydrofuran.

In another aspect of the method of synthesizing a compound of Formula (II), the method further comprises contacting the compound

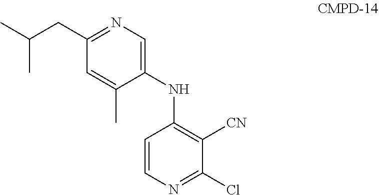 [SMILES: Cc1cc(CC(C)C)ncc1Nc1ccnc(Cl)c1C#N]

with a sulfur reagent and a base in a solvent to form the compound CMPD-13. In some aspects the sulfur reagent methyl 2-mercaptoacetate. In some aspects the base is NaOMe. In some aspects the solvent is methanol.

In another aspect of the method of synthesizing a compound of Formula (II), the method further comprises contacting the compound

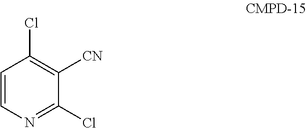

with the compound

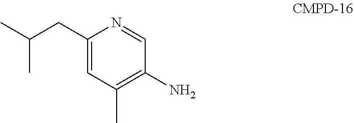

with a base, and an amine base in a solvent system to form the compound CMPD-14. In some aspects the base is K₂CO₃. In some aspects the amine base is N,N-diisopropylethylamine (DIPEA). In some aspects the solvent system is toluene/NMP.

In another aspect of the method of synthesizing a compound of Formula (II), the method further comprises contacting the compound

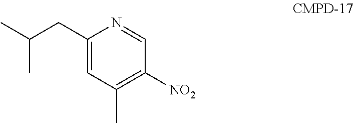

with a hydride source, a catalyst, and a solvent system to form the compound CMPD-16. In some aspects the hydride source is NaBH₄. In some aspects the catalyst is NiCl₂. In some aspects the solvent system is toluene/water.

In another aspect of the method of synthesizing a compound of Formula (II), the method further comprises contacting the compound

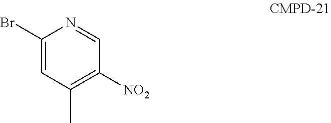

with a catalyst, phosphorus reagent, a boron reagent, a first base, a second base and a solvent system to form the compound CMPD-17. In some aspects the catalyst is Pd(OAc)₂. In some aspects the phosphorus reagent is di(1-adamantyl)-n-butylphosphine. In some aspects the boron reagent is isobutylboronic acid. In some aspects the first base is K₂CO₃. In some aspects the second base is potassium acetate (KOAc). In some aspects the solvent system is toluene/water.

In some aspects, the synthesis of a compound of Formula (II) is according to scheme 6.

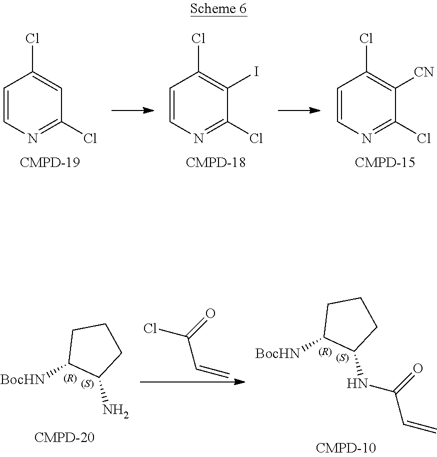

In another aspect of the method of synthesizing a compound of Formula (II), the method further comprises contacting the compound

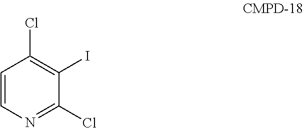

with a cyano source and a solvent to form the compound CMPD-15. In some aspects the cyano source is CuCN. In some aspects the solvent is butyronitrile.

In another aspect of the method of synthesizing a compound of Formula (II), the method further comprises contacting the compound

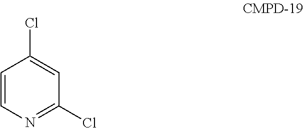

with a base and an electrophilic halogen in a solvent to form the compound CMPD-18. In some aspects the base is lithium diisopropylamine. In some aspects the solvent is tetrahydrofuran.

In another aspect of the method of synthesizing a compound of Formula (II), the

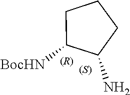

method further comprises contacting the compound CMPD-20 with a base,

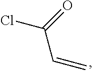

and a solvent to form the compound CMPD-10. In some aspects the base is NaHCO₃. In some aspects the solvent is 2-methyltetrahydrofuran.

In another aspect of the method of synthesizing a compound of Formula (I), the method comprises synthesis of CMPD-14 according to Scheme 7

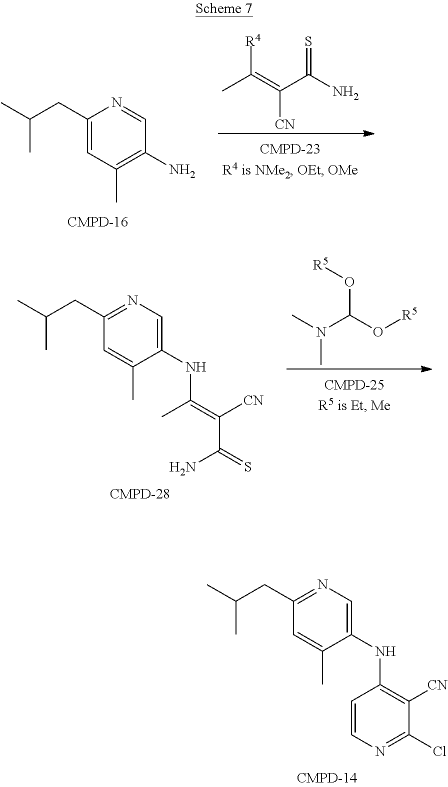

In another aspect of the method of synthesizing a compound of Formula (I), the method further comprises contacting the compound

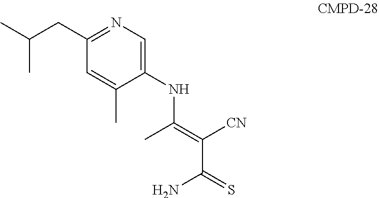 [SMILES: C/C(Nc1cnc(CC(C)C)cc1C)=C(/C#N)C(N)=S]

with compound

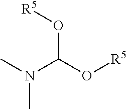

CMPD-25 and a base in a solvent to form the compound CMPD-14 wherein R⁵ is C₁₋₆alkyl. In some aspects C₁₋₆alkyl is methyl and ethyl. In some aspects CMPD-25 is selected from N,N-dimethylformamide dimethyl acetal and N,N-dimethylformamide diethyl acetal. In some aspects the base is selected from sodium hydroxide (NaOH), potassium phosphate tribasic (K₃PO₄), potassium carbonate (K₂CO₃), 1,8-diazabicyclo[5.4.0]undec-7-ene (DBU) and morpholine. In some aspects the solvent is selected from one or more solvents such as N,N-dimethylformamide (DMF), isopropyl acetate, 2-methyltetrahydrofuran (MeTHF), tetrahydrofuran (THF), acetonitrile (ACN). toluene, ethanol, tert-butyl methyl ether (TBME) and water.

In another aspect of the method of synthesizing a compound of Formula (I), the method further comprises contacting the compound

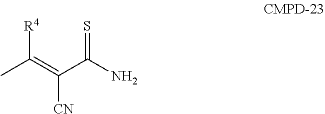

with compound

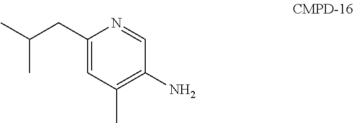

in a solvent to form the compound CMPD-28, wherein R⁴ is (C₁₋₆alkyl)₂N and C₁₋₆alkylO. In some aspects C₁₋₆alkyl is selected from methyl and ethyl. In some aspects the solvent is selected from one or more solvents such as N,N-dimethylformamide (DMF), 1-methyl-2-pyrrolidone (NMP), isopropyl acetate, 2-methyltetrahydrofuran (MeTHF), tetrahydrofuran (THF), acetonitrile (ACN), ethanol, and methanol.

In another aspect of the method of synthesizing a compound of Formula (I), the method further comprises synthesis of CMPD-11 according to Scheme 8.

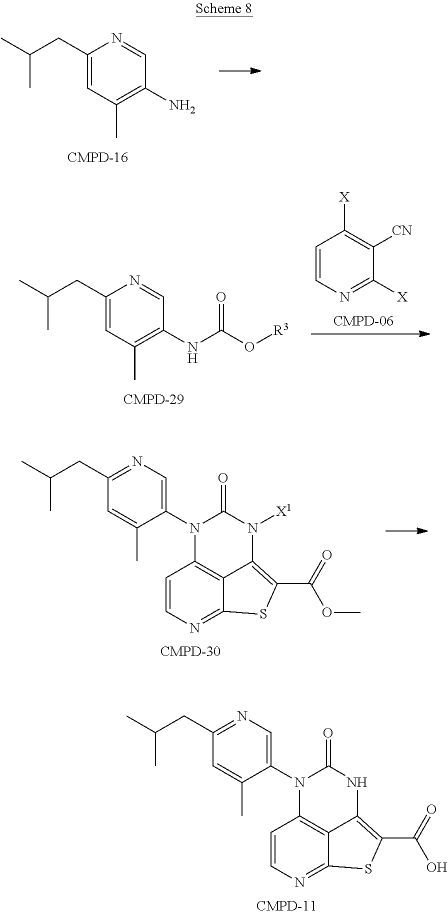

In another aspect of the method of synthesizing a compound of Formula (I), the method further comprises contacting the compound

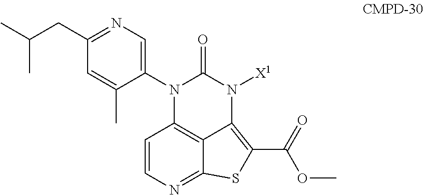

where X¹ is H or Na with a base and an ammonium salt in a solvent to form the compound CMPD-11. In some aspects the base is selected from potassium trimethylsilanolate (TMSOK), lithium hydroxide (LiOH), potassium hydroxide, (KOH), or sodium hydroxide (NaOH). In some aspects the ammonium salt is selected from tetramethyl ammonium bromide, tetraethylammonium bromide, tetraethyl ammonium chloride, tetrabutylammonium bromide (TBAB), tetrabutylammonium hydrogen sulfate, benzyltrimethylammonium bromide, benzyltrimethylammonium chloride, didodecyldimethylammonium bromide and Aliquat 336. More generally, those skilled in the art will recognize that alkylammonium salts and substituted alkylammonium salts may be used. In some aspects the solvent is tetrahydrofuran or methyltetrahydrofuran.

In another aspect of the method of synthesizing a compound of Formula (I), the

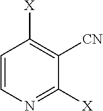

method further comprises contacting the compound CMPD-06 wherein X is selected from Cl and Br, with the compound

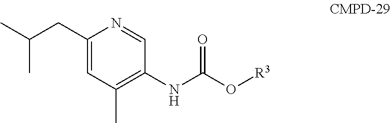

where R³ is C₁₋₆ alkyl and with a sulfur reagent, in the presence of one or more base and an ammonium salt in a solvent to form the compound CMPD-30, where X¹ is H or Na. In some aspects, the compond CMPD-30 where X¹ is H may be converted to CMPD-30 where X¹ is Na. More generally, those skilled in the art will recognize that X¹ may be a monovalent alkali metal or an ammonium. In some aspects X is Cl. In some aspects C₁₋₆ alkyl is selected from methyl, ethyl and tert-butyl. In some aspects the sulfur reagent is methyl 2-mercaptoacetate In some aspects the base is selected from one or more bases such as sodium hydroxide (NaOH), potassium hydroxide (KOH), potassium phosphate tribasic (K₃PO₄), sodium carbonate (Na₂CO₃), potassium carbonate (K₂CO₃), 1,8-diazabicyclo[5.4.0]undec-7-ene (DBU), 1,1,3,3-tetramethylguanidine (TMG), sodium methanolate (NaOMe), sodium tert-butoxide (t-BuONa). In some aspects the ammonium salt is selected from tetrabutylammonium bromide (TBAB), tetrabutylammonium chloride (TBAC), benzyltrimethylammonium chloride and tetramethylammonium chloride. More generally, those skilled in the art will recognize that alkylammonium salts and substituted alkylammonium salts may be used. In some aspects the solvent is selected from one or more solvents such as N,N-dimethylformamide (DMF), isopropyl acetate, 2-methyltetrahydrofuran (MeTHF), tetrahydrofuran (THF), acetonitrile (ACN). toluene, tetrahydrofuran (THF), methanol, isopropyl alcohol, tert-butyl methyl ether (TBME) and water.

In another aspect of the method of synthesizing a compound of Formula (I), the method further comprises contacting the compound

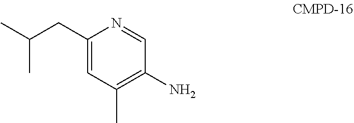

with an acylating agent in the presence or absence of a base or an acid in a solvent to form the compound CMPD-29, wherein R³ is C₁-6 alkyl. In some aspects C₁₋₆ alkyl is selected from methyl, ethyl and tert-butyl. In some aspects the acylating agent is selected from methyl chloroformate, dimethyl carbonate, ethyl chloroformate, diethyl carbonate, di-tert-butyl dicarbonate. In some aspects the acid is selected from acetic acid (AcOH), pivalic acid (PivOH), ammonium chloride (NH₄Cl), sodium hydrogen sulfate (NaHSO₄). In some aspects the base is selected from potassium phosphate tribasic (K₃PO₄), potassium carbonate (K₂CO₃), sodium carbonate (Na₂CO₃), cesium carbonate (Cs₂CO₃), sodium hydrogencarbonate (NaHCO₃) and sodium bis(trimethylsilyl)amide (NaHMDS), potassium tert-butoxide. In some aspects the solvent is selected from one or more solvents such as N,N-dimethylformamide (DMF), 1-methyl-2-pyrrolidone (NMP), isopropyl acetate, ethyl acetate (EtOAc), toluene, acetone, 2-methyltetrahydrofuran (MeTHF), tetrahydrofuran (THF), acetonitrile (ACN), isopropyl alcohol, ethanol, and methanol, and water. 4-Methyl-2-pentanone, tert-butyl methyl ether (TBME), cyclopentyl methyl ether (CPME).

### Methods of Converting to Single Atropisomer

The present disclosure is also directed to a method of isolating the P atropisomer from a mixture of M and P isomers of compound of Formula (II), comprising the steps of:
1) Dissolve the compound of Formula (II) in one or more solvents in a vessel to form a solution.
2) Optionally, heat the solution to a first temperature of about 75° C. to about 140° C. for a first time period of about 8 hours to about 12 hours, and cool the solution to a second temperature of about 20° C. to about 30° C.
3) Add water and wait for a second time period of about 2 hours to 24 hours to form a suspension. Optionally, seed with a compound of Formula P-(II) during this step. Optionally, cool the solution to a third temperature about 5° C. to about 15° C. for a third time period of about 6 hours to about 12 hours to form a suspension.
4) Filter the suspension to obtain the crystals of compound of Formula P-(II).
5) Collect the filtrate and heat it to a fourth temperature of about 75° C. to about 150° C. for a fourth time period of about about 5 minutes to about 1 hour.
6) Cool the filtrate to a fifth temperature of about 20° C. to about 30° C. 7) Repeat Steps 4-6 about 20-100 times until all the M isomer is converted to P isomer.
8) Collect the precipitate, wash, and dry the solid under humidified conditions.

In some embodiments, repeating Steps 4-6 can also be performed by adding the filtrate that is heated in Step 5 back to the vessel of Step 1, allowing it to cool and to form a suspension, and filtering the suspension to obtain the crystals of compound of Formula P-(II). This process is repeated 20-100 times until until all the M isomer is converted to P isomer.

In some aspectes the one or more solvents is selected from the group consisting of isopropanol, propylene carbonate, water, 1-propanol, 1-butanol, 2-butanol, isobutanol, propylene glycol, ethylene glycol, dimethylacetamide (DMA), benzyl alcohol, N-methyl-2-pyrrolidone (NMP), propylene glycol methyl ether, 2-ethoxyethanol, (s)-1,2-Butanediol, transcutol, ethylene glycol diacetate, and combinations thereof. In some aspectes the one or more solvents is 1-propanol. In some aspects the one or more solvents are 1-propanol and water.

In some aspects the first temperature is about 75° C. to about 140° C., about 80° C. to about 140° C., about 85° C. to about 140° C., about 90° C. to about 140° C., about 95° C. to about 140° C., about 75° C. to about 135° C., about 75° C. to about 130° C., about 75° C. to about 125° C., about 75° C. to about 120° C., about 75° C. to about 115° C., about 75° C. to about 110° C., about 75° C. to about 105° C., about 75° C. to about 100° C., about 75° C. to about 95° C., about 80° C. to about 135° C., about 85° C. to about 130° C., or about 90° C. to about 125° C. In some aspects the first temperature is about 75° C., about 80° C., about 85° C., about 90° C., about 95° C., about 100° C., about 105° C., about 110° C., about 115° C., about 120° C., about 125° C., about 130° C., about 135° C., or about 140° C. In some aspects the first temperature is about 95° C.

In some aspects the first time period is about 8 hours to about 12 hours, about 9 hours to about 12 hours, about 10 hours to about 12 hours, about 11 hours to about 12 hours, about 8 hours to about 11 hours, about 8 hours to about 10 hours, about 8 hours to about 9 hours, or about 9 hours to about 11 hours. In some aspects the first time period is about 8 hours, about 9 hours, about 10 hours, about 11 hours, or about 12 hours. In some aspects the first time period is about 10 hours.

In some aspects the second temperature is about 20° C. to about 30° C., about 25° C. to about 30° C. or about 20° C. to about 35° C. In some aspects the second temperature is about 25° C. In some aspects the second temperature is about 30° C. In some aspects the second temperature is about 35° C.

In some aspects the second time period is about 2 hours to about 24 hours, about 3 hours to about 24 hours, about 4 hours to about 24 hours, about 5 hours to about 24 hours, about 6 hours to about 24 hours, about 7 hours to about 24 hours, about 8 hours to about 24 hours, about 9 hours to about 24 hours, about 10 hours to about 24 hours, about 11 hours to about 24 hours, about 12 hours to about 24 hours, about 13 hours to about 24 hours, about 14 hours to about 24 hours, about 15 hours to about 24 hours, about 16 hours to about 24 hours, about 17 hours to about 24 hours, about 18 hours to about 24 hours, about 19 hours to about 24 hours, about 20 hours to about 24 hours, about 21 hours to about 24 hours, about 22 hours to about 24 hours, about 32 hours to about 24 hours, about 2 hours to about 23 hours, about 2 hours to about 22 hours, about 2 hours to about 21 hours, about 2 hours to about 20 hours, about 2 hours to about 19 hours, about 2 hours to about 18 hours, about 2 hours to about 17 hours, about 2 hours to about 16 hours, about 2 hours to about 15 hours, about 2 hours to about 14 hours, about 2 hours to about 13 hours, about 2 hours to about 12 hours, about 2 hours to about 11 hours, about 2 hours to about 10 hours, about 2 hours to about 9 hours, about 2 hours to about 8 hours, about 2 hours to about 7 hours, about 2 hours to about 6 hours, about 2 hours to about 5 hours, about 2 hours to about 4 hours, or about 2 hours to about 3 hours. In some aspects the second time period is about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some aspects the second time period is about 5 hours.

In some aspects the third temperature is about 5° C. to about 15° C., about 10° C. to about 15° C. or about 5° C. to about 10° C. In some aspects the third temperature is about 5° C. In some aspects the third temperature is about 10° C. In some aspects the third temperature is about 15° C.

In some aspects the third time period is about 6 hours to about 12 hours, about 7 hours to about 12 hours, about 8 hours to about 12 hours, about 9 hours to about 12 hours, about 10 hours to about 12 hours, about 11 hours to about 12 hours, about 6 hours to about 11 hours, about 6 hours to about 10 hours, about 6 hours to about 9 hours, about 6 hours to about 8 hours, about 6 hours to about 7 hours, about 7 hours to about 11 hours, or about 8 hours to about 10 hours. In some aspects the fourth time period is about 6 hours, about 7 hours, about 8 hours, about 9 hours, about 10 hours, about 11 hours, or about 12 hours. In some aspects the third time period is about 8 hours.

In some aspects the fourth temperature is about 75° C. to about 150° C., about 80° C. to about 150° C., about 85 to about 150° C., about 95° C. to about 150° C., about 100° C. to about 150° C., about 105° C. to about 150° C., about 110° C. to about 150° C., about 115° C. to about 150° C., about 120° C. to about 150° C., about 125° C. to about 150° C., about 130° C. to about 150° C., about 135° C. to about 150° C., about 140° C. to about 150° C., about 145° C. to about 150° C., about 75° C. to about 145° C., about 75° C. to about 140° C., about 75° C. to about 135° C., about 75° C. to about 130° C., about 75° C. to about 125° C., about 75° C. to about 120° C., about 75° C. to about 115° C., about 75° C. to about 110° C., about 75° C. to about 105° C., about 75° C. to about 100° C., about 75° C. to about 95° C., about 75° C. to about 90° C., about 75° C. to about 85° C., about 75° C. to about 80° C., about 75° C. to about 135° C., about 80° C. to about 130° C., about 85° C. to about 125° C., about 90° C. to about 120° C., about 95° C. to about 115° C., or about 100° C. to about 110° C. In some aspects the fourth temperature is about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., or about 150° C.

In some aspects the fourth time period is about 5 minutes to about 1 hour, about minutes to about 1 hour, about 15 minutes to about 1 hour, about 20 minutes to about 1 hour, about 25 minutes to about 1 hour, about 30 minutes to about 1 hour, about 35 minutes to about 1 hour, about 40 minutes to about 1 hour, about 45 minutes to about 1 hour, about 50 minutes to about 1 hour, about 55 minutes to about 1 hour, about 10 minutes to about 55 minutes, about 10 minutes to about 50 minutes, about 10 minutes to about 45 minutes, about 10 minutes to about 40 minutes, about 10 minutes to about 35 minutes, about 10 minutes to about 30 minutes, about 10 minutes to about 25 minutes, about 10 minutes to about 10 minutes, or about 10 minutes to about 15 minutes. In some aspects the fourth time period is about 10 minutes, about 15 minutes, about 20 minutes, about 25 minutes, about 30 minutes, about 35 minutes, about 40 minutes, about 45 minutes, about 50 minutes, about 55 minutes, or about 1 hour.

In some aspects the fifth temperature is about 20° C. to about 30° C., about 25° C. to about 30° C. or about 20° C. to about 35° C. In some aspects the fifth temperature is about 25° C. In some aspects the second temperature is about 30° C. In some aspects the second temperature is about 35° C.

In aspects, steps 4-6 are repeated about 20 times, about 25 times, about 30 times, about 35 times, about 40 times, about 45 times, about 50 times, about 55 times, about 60 times, about 65 times, about 70 times, about 75 times, about 80 times, about 85 times, about 90 times, about 95 times, about 100 times, or until all the M isomer is converted to P isomer.

The present disclosure is also directed to a method of converting a compound of Formula (II) to compound of Formula P-(II) comprising the steps of:
1) Dissolve the compound of Formula (II) in one or more solvents in a vessel to form a solution,
2) Heating the solution to a first temperature of about 75° C. to about 120° C. and hold at the first temperature for a first time period of about 4 hours to about 8 hours followed by cooling the solution to a second temperature of about 60° C. to about 100° C.,
3) Add water and to solution a compound of Formula (II), followed by cooling the solution to third temperature about 10° C. to about 30° C. over a second time period of about 2 hours to about 8 hours and waiting for a third time period of about 3 hours to about 9 hours to form a suspension,
4) Filter the suspension to obtain the crystals of compound of Formula P-(II).
5) Collect the filtrate and heat it to a fourth temperature of about 110° C. to about 150° C. for a fourth time period of about 1 hour to about 5 hours,
6) Cool the filtrate to a fifth temperature of about 20° C. to about 30° C.
7) Repeat steps 4-6 10-100 times.
8) Collect the precipitate, wash, and dry the solid under humidified conditions.

In some aspects the one or more solvents is selected from the group consisting of isopropanol, propylene carbonate, water, 1-propanol, 1-butanol, 2-butanol, isobutanol, propylene glycol, ethylene glycol, dimethylacetamide (DMA), benzyl alcohol, N-methyl-2-pyrrolidone (NMP), propylene glycol methyl ether, 2-ethoxyethanol, (s)-1,2-Butanediol, transcutol, ethylene glycol diacetate, and combinations thereof. In some aspects the one or more solvents is propylene carbonate. In some aspects the one or more solvents are propylene carbonate and water.

In some aspects the first temperature is about 80° C. to about 120° C., about 85° C. to about 120° C., about 90° C. to about 120° C., about 95° C. to about 120° C., about 100° C. to about 120° C., about 105° C. to about 120° C., about 110° C. to about 120° C., about 115° C. to about 120° C., about 75° C. to about 115° C., about 75° C. to about 110° C., about 75° C. to about 105° C., about 75° C. to about 100° C., about 75° C. to about 95° C., about 75° C. to about 90° C., about 75° C. to about 85° C., about 75° C. to about 80° C., about 80° C. to about 115° C., about 85° C. to about 110° C., or about 90° C. to about 100° C. In some aspects the first temperature is about about 75° C., about 80° C., about 85° C., about 90° C., about 95° C., about 100° C., about 105° C., about 110° C., about 115° C., or about 120° C. In some aspects the first temperature is about 100° C.

In some aspects the first time period is about 5 hours to about 8 hours, about 6 hours to about 8 hours, about 7 hours to about 8 hours, about 4 hours to about 7 hours, about 4 hours to about 6 hours, about 4 hours to about 5 hours, or about 5 hours to about 7 hours. In some aspects the first time period is about 4 hours, about 5 hours, about 7 hours, or about 8 hours. In some aspects the first time period is about 6 hours.

In some aspects the second temperature is about 65° C. to about 100° C., about 70° C. to about 100° C., about 75° C. to about 100° C., about 80° C. to about 100° C., about 85° C. to about 100° C., about 90° C. to about 100° C., about 95° C. to about 100° C., about 65° C. to about 95° C., about 65° C. to about 90° C., about 65° C. to about 85° C., about 65° C. to about 80° C., about 65° C. to about 75° C., about 65° C. to about 70° C., about 70° C. to about 95° C., about 75° C. to about 90° C., or about 80° C. to about 85° C. In some aspects the second temperature is about 65° C., about 70° C., about 75° C., about 80° C., about 85° C., about 90° C., about 95° C., about 100° C., about 105° C., about 110° C., about 115° C., or about 120° C. In some aspects the second temperature is about 80° C.

In some aspects the third temperature is about 10° C. to about 30° C., about 15° C. to about 30° C., about 20° C. to about 30° C., about 25° C. to about 30° C., about 10° C. to about 25° C., about 10° C. to about 20° C., about 10° C. to about 15° C., or about 15° C. to about 25° C. In some aspects the third temperature is about 10° C., about 15° C., about 20° C., about 25° C., or about 30° C. In some aspects the third temperature is about 25° C.

In some aspects the second time period is about 3 hours to about 8 hours, about 4 hours to about 8 hours, about 5 hours to about 8 hours, about 6 hours to about 8 hours, about 7 hours to about 8 hours, about 2 hours to about 7 hours, about 2 hours to about 6 hours, about 2 hours to about 5 hours, about 2 hours to about 4 hours, about 2 hours to about 3 hours, about 3 hours to about 7 hours, or about 4 hours to about 6 hours. In some aspects the second time period is about 2 hours, about 3 hours, about 4 hours, about 5 hours, about 6 hours, about 7 hours, or about 8 hours. In some aspects the second time period is about 4 hours.

In some aspects the third time period is about 4 hours to about 9 hours, about 5 hours to about 9 hours, about 6 hours to about 9 hours, about 7 hours to about 9 hours, about 8 hours to about 9 hours, about 3 hours to about 8 hours, about 3 hours to about 7 hours, about 3 hours to about 6 hours, about 3 hours to about 5 hours, about 3 hours to about 4 hours, about 3 hours to about 7 hours, or about 4 hours to about 6 hours. In some aspects the third time period is about 3 hours, about 4 hours, about 5 hours, about 6 hours, about 7 hours, about 8 hours, or about 9 hours. In some aspects the third time period is about 5 hours.

In some aspects the fourth temperature is about 115° C. to about 150° C., about 120° C. to about 150° C., about 125° C. to about 150° C., about 130° C. to about 150° C., about 135° C. to about 150° C., about 140° C. to about 150° C., about 145° C. to about 150° C., about 110° C. to about 145° C., about 110° C. to about 140° C., about 110° C. to about 135° C., about 110° C. to about 130° C., about 110° C. to about 125° C., about 110° C. to about 120° C., about 110° C. to about 115° C., about 115° C. to about 145° C., about 120° C. to about 140° C., or about 125° C. to about 135° C. In some aspects the fourth temperature is about 110° C., about 115° C., about 120° C., about 125° C., about 130° C., about 135° C., about 140° C., about 145° C., or about 150° C. In some aspects the fourth temperature is about 130° C.

In some aspects the fourth time period is about 35 hours to about 70 hours, about 40 hours to about 70 hours, about 45 hours to about 70 hours, about 50 hours to about 70 hours, about 55 hours to about 70 hours, about 60 hours to about 70 hours, about 65 hours to about 70 hours, about 30 hours to about 65 hours, about 30 hours to about 60 hours, about 30 hours to about 55 hours, about 30 hours to about 50 hours, about 30 hours to about 45 hours, about 30 hours to about 40 hours, about 30 hours to about 35 hours, about 35 hours to about 65 hours, about 40 hours to about 60 hours, or about 45 hours to about 55 hours. In some aspects the fourth time period is about 30 hours, about 35 hours, about 40 hours, about 45 hours, about 50 hours, about 55 hours, about 60 hours, about 65 hours, or about 70 hours. In some aspects the fourth time period is about 52 hours.

In some aspects the fifth temperature is about 10° C. to about 30° C., about 15° C. to about 30° C., about 20° C. to about 30° C., about 25° C. to about 30° C., about 10° C. to about 25° C., about 10° C. to about 20° C., about 10° C. to about 15° C., or about 15° C. to about 25° C. In some aspects the fifth temperature is about 10° C., about 15° C., about 20° C., about 25° C., or about 30° C. In some aspects the third temperature is about 25° C.

The present disclosure is also directed to a method of converting a compound of Formula (II) to compound of Formula P-(II) comprising the steps of:
1) Dissolving the compound of Formula (II) in one or more solvents to form a solution,
2) Heating the solution to a first temperature of about 50° C. to about 100° C. for a time period of about 30 hours to about 80 hours,
3) Cool the solution to second temperature of about 0° C. to about 20° C.,
4) Collect the formed crystals of the compound of Formula P-(II) by filtration.

In some aspects the one or more solvents is selected from the group consisting of isopropanol, propylene carbonate, water, 1-propanol, 1-butanol, 2-butanol, isobutanol, propylene glycol, ethylene glycol, dimethylacetamide (DMA), benzyl alcohol, N-methyl-2-pyrrolidone (NMP), propylene glycol methyl ether, 2-ethoxyethanol, (s)-1,2-Butanediol, transcutol, ethylene glycol diacetate, and combinations thereof. In some aspects the one or more solvents are isopropanol and water.

In some aspects the first temperature is about 55° C. to about 100° C., 60° C. to about 100° C., 65° C. to about 100° C., about 70° C. to about 100° C., about 75° C. to about 100° C., about 80° C. to about 100° C., about 85° C. to about 100° C., about 90° C. to about 100° C., about 95° C. to about 100° C., about 50° C. to about 95° C., about 50° C. to about 90° C., about 50° C. to about 85° C., about 50° C. to about 80° C., about 50° C. to about 75° C., about 50° C. to about 70° C., about 50° C. to about 65° C., 50° C. to about 60° C., 50° C. to about 55° C., about 55° C. to about 95° C., about 60° C. to about 90° C., about 65° C. to about 85° C., about 70° C. to about 90° C., or about 75° C. to about 85° C. In some aspects the first temperature is about 50° C., about 55° C., about 60° C., about 65° C., about 70° C., about 75° C., about 80° C., about 85° C., about 90° C., about 95° C., or about 100° C. In some aspects the first temperature is about 80° C.

In some aspects the first time period is about 35 hours to about 80 hours, about 40 hours to about 80 hours, about 45 hours to about 80 hours, about 50 hours to about 80 hours, about 55 hours to about 80 hours, about 60 hours to about 80 hours, about 65 hours to about 80 hours, about 70 hours to about 80 hours, about 75 hours to about 80 hours, about 30 hours to about 75 hours, about 30 hours to about 70 hours, about 30 hours to about 65 hours, about 30 hours to about 60 hours, about 30 hours to about 55 hours, about 30 hours to about 50 hours, about 30 hours to about 45 hours, about 30 hours to about 40 hours, about 30 hours to about 35 hours, about 35 hours to about 75 hours, about 40 hours to about 70 hours, about 45 hours to about 65 hours, or about 50 hours to about 60 hours. In some aspects the first time period is about 35 hours, about 40 hours, about 45 hours, about 50 hours, about 55 hours, about 60 hours, about 65 hours, about 70 hours, about 75 hours, or about 80 hours.

In some aspects the second temperature is about 5° C. to about 20° C., about 10° C. to about 20° C., about 15° C. to about 20° C., about 0° C. to about 15° C., about 0° C. to about 10° C., about 0° C. to about 5° C., or about 5° C. to about 15° C. In some aspects the second temperature is about 0° C., about 5° C., about 10° C., about 15° C., or about 20° C. In some aspects the second temperature is about 10° C.

### Crystalline Form B

The present disclosure includes embodiments directed to crystalline Form B of Formula P-(II) dihydrate:

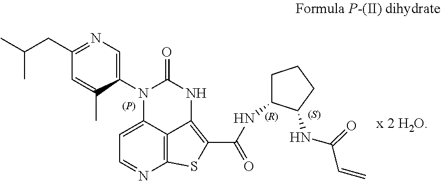

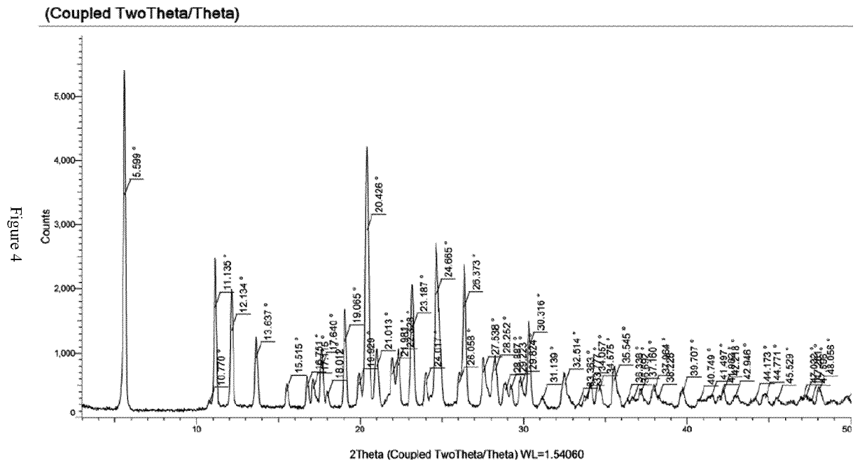

In some embodiments, crystalline Form B is characterized by a XRPD pattern having a peak expressed in degrees 2θ (±0.2) at about 5.599°. In some embodiments, crystalline Form B is further characterized by a XRPD pattern having a peak expressed in degrees 2θ (±0.2) at about 20.426°. In some embodiments, crystalline Form B is further characterized by a XRPD pattern having a peak expressed in degrees 2θ (±0.2) at about 24.665°. In some embodiments, crystalline Form B is further characterized by a XRPD pattern having peaks expressed in degrees 2θ (±0.2) at about 11.135° and about 26.373°. In some embodiments, crystalline Form B is further characterized by a XRPD pattern having peaks expressed in degrees 2θ (±0.2) at about 12.134°, about 23.187°, about 19.065°, and about 30.316°. In some embodiments, crystalline Form B is characterized by a XRPD pattern substantially as shown in FIG. 4.

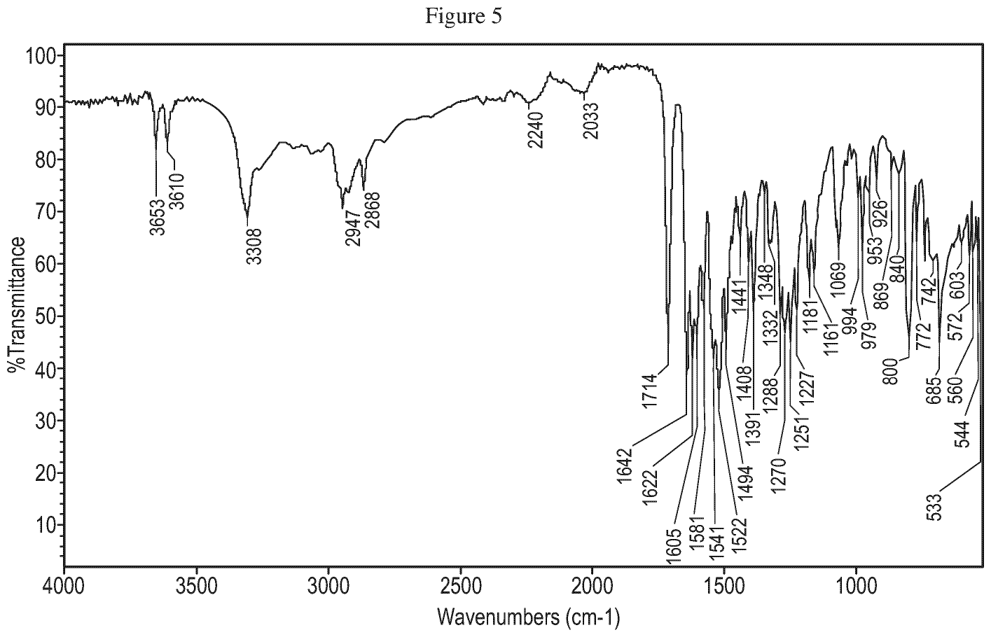

In some embodiments, crystalline Form B is characterized by an IR peak at about 1522 cm⁻¹. In some embodiments, crystalline Form B is further characterized by an IR peak at about 1714 cm⁻¹. In some embodiments, crystalline Form B is further characterized by an IR peak at about 1642 cm⁻¹ and about 1622 cm⁻¹. In some embodiments, crystalline Form B is further characterized by an IR peak at about 1270 cm⁻¹ and about 1251 cm⁻¹. In some embodiments, crystalline Form B is further characterized by an IR peak at about 1541 cm⁻¹ and about 1494 cm⁻¹. In some embodiments, crystalline Form B is further characterized by an IR pattern substantially as shown in FIG. 5.

In some embodiments, crystalline Form B may also be characterized by one or more of 1) a DSC thermograms utilizing standard pan conditions exhibiting an endotherm at about 196.8° C.; 2) a DSC thermograms utilizing Tzero hermetically sealed pan conditions exhibiting a first endotherm at about 141.8° C. and a second endotherm at about 155.9° C.; and 3) a water loss as measured by thermogravimetric analysis of about 6.5 wt. %.

### Compounds

The present disclosure is also directed to compound having the strucure:

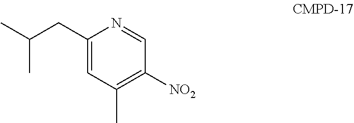

### EXAMPLES

### Example 1: Synthesis of 2,4-dichloro-3-iodopyridine (CMPD-18)

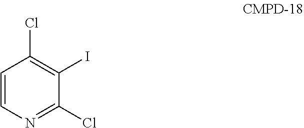

In a reactor, 100 g 2,4-dichloropyridine (100 g, 0.68 mol) was added to THF (1000 mL). The reaction mixture was cooled to −78° C. A solution of LDA (0.72 mol) was slowly added keeping reaction temperature below −70° C. Then a solution of I₂ (208 g, 0.82 mol) in THF (1200 mL) was slowly added. The reaction mixture was warmed to 10° C. and quenched with 5% NaHSO₃ aqueous solution (400 mL). The organic layer was then switched to PrCN and then washed with water (500 mL). The final solution in PrCN (400 mL) can be used directly for next step (85% assay yield). ¹H NMR (400 MHZ, CDCl₃): δ 8.22 (d, J=5.2 Hz, 1H), 7.30 (d, J=5.2 Hz, 1H).

### Example 2: Synthesis of 2,4-dichloronicotinonitrile (CMPD-15)

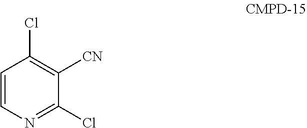

In a reactor with the solution of 2,4-dichloro-3-iodopyridine (83.1 g, 0.30 mol) in PrCN (400 mL), CuCN (51.6 g, 0.58 mol) was added. The reaction mixture was heated to 95-100° C. for 10 hours. The mixture was filtered and washed with PrCN (80 mL). The filtrate was solvent swap to n-BuOH then toluene. The final solution was filtered. The organic layer was washed with 4% EDTA solution (400 mL) and water (400 mL) before concentrated to 2-3 V. Heptane (830 mL) was added to crystallize the desired product (41.0 g, 76.5% yield). ¹H NMR (400 MHZ, DMSO-D₆): δ 8.67 (d, J=5.5 Hz, 1H), 7.92 (d, J=5.5 Hz, 1H).

### Example 3: Synthesis of tert-butyl ((1R,2S)-2-acrylamidocyclopentyl)carbamate (CMPD-10)

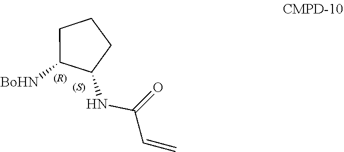

In a reactor, tert-butyl ((1R,2S)-2-aminocyclopentyl)carbamate (12.9 kg, 64.4 mol) was added to MeTHF (193.5 L). BHT (64.5 g, 0.29 mol) and a solution of 7% NaHCO₃ (193.5 L) was added and the mixture was cooled to 10° C. Acryloyl chloride (6.88 kg, 76.0 mol) was added dropwise to the mixture maintaining internal temperature below 10° C. After the reaction completed, the layer was separated. The organic layer was washed with 10% Na₂SO₄ (70 L) then evaporated to ˜64.5 L. Heptane (64.5 L) was added to crystallize out the product. After filtration, the product was isolated as a solid (14.7 kg, 90% yield). MS (ESI): mass calcd. for C₁₃H₂₂N₂O₃, 254.32; m/z found, 255.0 [M+H]⁺. ¹H NMR (400 MHZ, DMSO-D₆): δ 7.68-7.50 (m, 1H), 6.47-6.31 (m, 1H), 6.28-6.16 (m, 1H), 6.12-5.96 (m, 1H), 5.60-5.46 (m, 1H), 4.13-3.98 (m, 1H), 3.88-3.68 (m, 1H), 1.87-1.58 (m, 3H), 1.55-1.37 (m, 3H), 1.32 (s, 9H).

The title compound was also prepared in an analoguous manner using 3-chloropropionyl chloride. In a reactor, tert-butyl ((1R,2S)-2-aminocyclopentyl)carbamate (50 g, 249.7 mol) was added to MeTHF (400 mL). Triethylamine (41.8 mL, 300 mmol) was added and the mixture was cooled to 10° C. A solution of 3-chloropropionyl chloride (27.4 mL, 287.1 mmol) in MeTHF (200 mL) was added dropwise to the mixture maintaining internal temperature below 10° C. After the reaction completed, water (200 mL) was charged and the aqueous layer was discarded. Water (200 mL) and sodium hydroxide (50 wt %, 50 g, 624.1 mmol) were added and the mixture was warmed up to 60° C. After the reaction completed, the layers were separated at 60° C. The organic layer was washed at 60° C. with 10% Na₂SO₄ aqueous solution (200 mL). MeTHF (250 mL) was added and the organic layer wa dried azeotrpically by distillation. Heptane (150 mL) was charged and the reaction mixture was cooled down to 20° C. to crystallize the desired product (47.6 g, 74.5% yield).

### Example 4: Synthesis of 2-isobutyl-4-methyl-5-nitropyridine (CMPD-17)

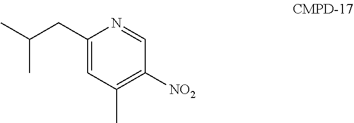

In an inert reactor, Pd(OAc)₂ (2.06 g, 9.2 mmol), Catacxium A (13.60 g, 18.4 mmol) was added into a mixture of toluene (2.0 L) and water (500 mL). After 30 min, 2-bromo-4-methyl-5-nitropyridine (200 g, 0.92 mol), isobutylboronic acid (140.7 g, 1.38 mol), K₂CO₃ (381 g, 2.76 mol) and KOAc (9.03 g, 92 mmol) were subsequently added. The reaction mixture was heated to 100° C. for 16 hours. The mixture was cooled down to 25° C. and water layer was discarded. Organic layer was washed with NaOH 30% solution (1000 mL). After filtration, the organic layer containing the desired product was used directly for the next step. (95% assay yield). ¹H NMR (400 MHZ, CDCl₃): δ 9.1 (s, 1H), 7.09 (s, 1H), 2.69 (d, J=7.6 Hz, 2H) 2.63 (s, 3H), 2.14 (m, 1H), 0.94 (d, J=6.8 Hz, 6H).

### Example 5: Synthesis of 6-isobutyl-4-methylpyridin-3-amine (CMPD-16)

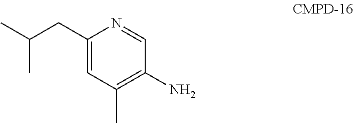

In a reactor, NiCl₂·6H₂O (2.07 g, 8.7 mmol) was added into water (680 mL). A 2.1 L toluene solution of 2-isobutyl-4-methyl-5-nitropyridine (169.5 g, 0.87 mol) was added. The reaction mixture was heated to 50° C. NaBH₄ (65.0 g, 1.74 mol) was added portion-wise keeping internal temperature between 50-60° C. The reaction mixture was heated at 60° C. for 17 hours. The organic layer was separated and filtered. The filtrate was washed with water (340 mL) to give a solution of the desired product (90-95% assay yield). This solution was solvent switched to NMP to prepare for the next step. ¹H NMR (400 MHZ, DMSO-D₆): δ 7.81 (s, 1H), 6.74 (s, 1H), 4.80 (s, 2H), 2.37 (d, J=7.2 Hz, 2H) 2.04 (s, 3H), 1.91 (m, 1H), 0.83 (d, J=6.4 Hz, 6H).

### Example 6: Synthesis of 2-chloro-4-((6-isobutyl-4-methylpyridin-3-yl)amino) nicotinonitrile (CMPD-14)

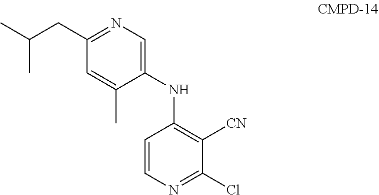 [SMILES: Cc1cc(CC(C)C)ncc1Nc1ccnc(Cl)c1C#N]

In an inert reactor, a solution of 6-isobutyl-4-methylpyridin-3-amine (30.0 g, 183 mmol) in toluene (600 mL), 2,4-dichloronicotinonitrile (229 mmol), DIPEA (183 mmol), K₂CO₃ (183 mmol) and LiCl (275 mmol) were added together in NMP (150 mL). The mixture was heated to 120° C. for 40 hours. Once the reaction was finished, the mixture was cooled down and can be used for the next step (89% assay yield). ¹H NMR (400 MHZ, CDCl₃): δ 8.37 (s, 1H), 8.04 (d, J=6 Hz, 1H), 7.10 (s, 1H), 6.72 (s, 1H), 6.26 (d, J=6 Hz, 1H), 2.65 (d, J=7.2 Hz, 2H), 2.23 (s, 3H), 2.10 (m, 1H), 0.95 (d, J=6.4 Hz, 6H).

### Example 7: Synthesis of Methyl 3-amino-4-((6-isobutyl-4-methylpyridin-3-yl)amino)thieno[2,3-b]pyridine-2-carboxylate (CMPD-13)

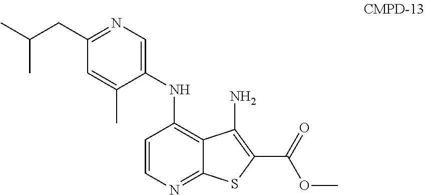 [SMILES: COC(=O)c1sc2nccc(Nc3cnc(CC(C)C)cc3C)c2c1N]

To a solution of 2-chloro-4-((6-isobutyl-4-methylpyridin-3-yl)amino) nicotinonitrile (27.5 g, 91.7 mmol) in NMP (130 mL), methyl 2-mercaptoacetate (34.8 g, 328 mmol) was added. A solution of NaOMe (12.3 g, 228 mmol) in MeOH was added slowly keeping reaction temperature below 30° C. After the reaction is finished, water (275 mL) was added. The crude product was isolated by filtration. The obtained solid was slurried in water (275 mL) and filtered to give the targeted product 24.3 g, 72% yield). ¹H NMR (400 MHZ, DMSO-D₆): δ 8.36 (s, 1H), 8.34 (s, 1H), 8.11 (d, J=5.2 Hz, 1H), 7.22 (s, 1H), 7.18 (s, 2H), 5.98 (d, J=5.6 Hz, 1H), 3.79 (s, 3H), 2.59 (d, J=6.8 Hz, 2H), 2.13 (s, 3H), 2.08 (m, 1H), 0.90 (d, J=6.4 Hz, 6H).

### Example 8: Synthesis of Methyl 5-(6-isobutyl-4-methylpyridin-3-yl)-4-oxo-4,5-dihydro-3H-1-thia-3,5,8-triazaacenaphthylene-2-carboxylate (CMPD-12)

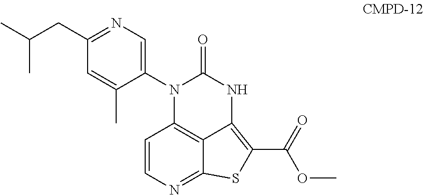 [SMILES: COC(=O)c1sc2nccc3c2c1NC(=O)N3c1cnc(CC(C)C)cc1C]

In an inert reactor, methyl 3-amino-4-((6-isobutyl-4-methylpyridin-3-yl)amino)thieno[2,3-b]pyridine-2-carboxylate (46.5 kg, 125.5 mol) was dissolved in THF (232 L). CDI (20.4 kg, 251 mol) was added. The mixture was stirred for 6-10 hours at 60° C. When the reaction is finished, water (465 L) was slowly dosed at 25° C. The desired product (44.4 kg, 89.2% yield) was obtained after filtration. ¹H NMR (400 MHZ, DMSO-D₆): δ 10.79 (s, 1H), 8.47 (s, 1H), 8.38 (d, J=5.6 Hz, 1H), 7.36 (s, 1H), 5.97 (d, J=5.6 Hz, 1H), 3.85 (s, 3H), 2.63 (d, J=7.2 Hz, 2H), 2.13 (s, 3H), 2.11 (m, 1H), 0.94 (d, J=6.8 Hz, 6H).

### Example 9: Synthesis of 5-(6-isobutyl-4-methylpyridin-3-yl)-4-oxo-4,5-dihydro-3H-1-thia-3,5,8-triazaacenaphthylene-2-carboxylic acid (CMPD-11)

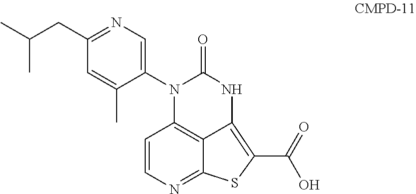 [SMILES: Cc1cc(CC(C)C)ncc1N1C(=O)Nc2c(C(=O)O)sc3nccc1c23]

In an inert reactor, methyl 5-(6-isobutyl-4-methylpyridin-3-yl)-4-oxo-4,5-dihydro-3H-1-thia-3,5,8-triazaacenaphthylene-2-carboxylate (36.0 kg, 90.8 mol), TMSOK (34.9 kg, 272 mol) and TBAB (7.0 kg, 45.4 mol) was charged into THF (720 L). The mixture was heated to 60° C. for 10-16 hours. Once full conversion was achieved, the reaction was cooled down and adjusted to pH 4-5 by adding 0.5 M H₂SO₄ solution (900 L). THF was removed under vacuum at <40° C. Water (180 L) was added and the product was obtained by filtration after washes with water and MTBE. The solid was dried in the oven at 50° C. to give desired product (32.9 kg, 98% yield). MS (ESI): mass calcd. for C₁₉H₁₈N₄O₃S, 382.11 m/z found, 383.1 [M+H]⁺.

### Example 10: Synthesis of N-((1R,2S)-2-acrylamidocyclopentyl)-5-(6-isobutyl-4-methylpyridin-3-yl)-4-oxo-4,5-dihydro-3H-1-thia-3,5,8-triazaacenaphthylene-2-carboxamide (Formula (II))

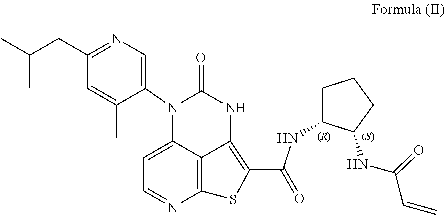

In a separated reactor, tert-butyl ((1R,2S)-2-acrylamidocyclopentyl)carbamate (730 g, 2.87 mol) was added to DCM (10 L). MSA (506.9 g, 5.22 mol) was slowly added at 25-35° C. The solution was stirred and used directly in the next step. In a solution of 5-(6-isobutyl-4-methylpyridin-3-yl)-4-oxo-4,5-dihydro-3H-1-thia-3,5,8-triazaacenaphthylene-2-carboxylic acid (1.0 kg, 2.61 mol) in DCM (10 L), EDCI (751 g, 3.92 mol) and HOBt (530 g, 3.92 mol) were added at 0-10° C. After 2 hours, the previous solution of diamine and BHT (5.0 g) were added. DIPEA (13.1 mol) was then slowly dosed keeping reaction temperature below 10° C. When the reaction is finished, the mixture was washed with water (5 L), 2 times citric acid 5% (5 L), NaHCO₃5% (5 L) and NaHSO₄ 5% (10 L). The organic layer (91% assay yield) was solvent switched to 1-propanol and used as such for the next step.

### Example 11: Synthesis of tert-butyl ((1R,2S)-2-(3-chloropropanamido)cyclopentyl)carbamate (CMPD-31)

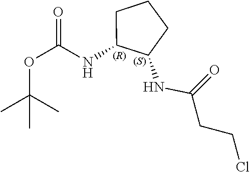

In a reactor, tert-butyl ((1R,2S)-2-aminocyclopentyl)carbamate (3.0 g, 15.0 mmol) was added to MeTHF (30 mL). Aqueous sodium hydrogen carbonate (7.0 wt %, 45 mL, 37.5 mmol) was charged and the mixture was cooled to 10° C. A solution of 3-chloropropionyl chloride (2.19 g, 17.2 mmol) in MeTHF (15 mL) was added dropwise to the mixture maintaining internal temperature below 10° C. After the reaction completed, the layers were separated. The organic layer was washed with aqueous sodium hydrogen carbonate (7.0 wt %, 45 mL). The organic layer was concentrated by distillation to crystallize the desired product (4.46 g, 100%). ¹H NMR 4 Hz,00 MHz, DMSO-D₆): δ 7.56 (d, J: =7.6 Hz, 1H), 6.28 (d, J: =7.6 Hz, 1H), 4.04 (m, 1H), 3.85-3.70 (m, 1H), 3.76 (td, J: =6.8, 1.6 Hz, 2H), 2.61-2.52 (m, 2H), 1.86-1.71 (m, 2H), 1.71-1.60 (m, 1H), 1.55-1.37 (m, 3H), 1.36 (s, 9H).

### Example 12: Synthesis of (E)-2-cyano-3-((6-isobutyl-4-methylpyridin-3-yl)amino) but-2-enethioamide (CMPD-28)

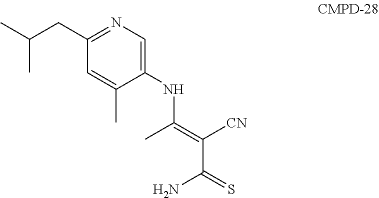 [SMILES: C/C(Nc1cnc(CC(C)C)cc1C)=C(/C#N)C(N)=S]

In a reactor, 2-cyano-3-ethoxybut-2-enethioamide (513 g, 3.02 mol) was added to tetrahydrofuran (4.5 L) at 20° C. 6-isobutyl-4-methylpyridin-3-amine (449 g, 2.74 mol) was added at 20° C. and the reaction mixture was warmed up to 60° C. After the reaction completed, the solvent was switched to isopropanol (2.25 L) and water (4.5 L) was added to crystallize the desired product (706.8 g, yield 89.4%). ¹H NMR (400 MHZ, CDCl₃): δ 14.20 (s, 1H), 8.27 (s, 1H), 7.08 (s, 1H), 7.00 (brs, 1H), 6.76 (brs, 1H), 2.65 (d, J=7.2 Hz, 2H), 2.32 (s, 3H), 2.25 (s, 3H), 2.10 (sept., J=6.4 Hz, 1H), 0.94 (d, J=6.4 Hz, 6H).

### Example 13: Synthesis of 4-((6-isobutyl-4-methylpyridin-3-yl)amino)-2-mercaptonicotinonitrile (CMPD-29)

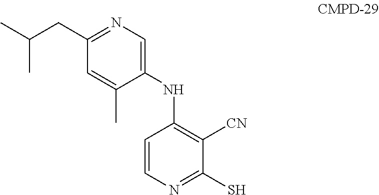 [SMILES: Cc1cc(CC(C)C)ncc1Nc1ccnc(S)c1C#N]

In a reactor, 2-cyano-3-((6-isobutyl-4-methylpyridin-3-yl)amino) but-2-enethioamide (711 g, 2.47 mol) was added to 2-methyltetrahydrofuran (7.1 L) and the reaction mixture was warmed up to 40° C. N,N-dimethylformamide diethyl acetal (909 g, 6.17 mol) was charged and the reaction mixture was stirred for 17 h. 8.7% K₂CO₃ (3.55 L, 2.47 mol) was added to the reaction mixture at 40° C. After the reaction completed, the reaction mixture was cooled down to 0° C. and glacial acetic acid (420 mL, 7.34 mol) was added to reach pH=5-6. The reaction mixture was warmed up to 20° C. and the layers were separated. The organic layer was washed with 4% Na₂SO₄ (3.55 L) then 2-methyltetrahydrofuran (5.0 L) was removed by distillation. Tert-butyl methyl ether (7.1 L) was added and the reaction mixture was cooled down to 20° C. to crystallize the desired product (653 g, yield 88.9%). ¹H NMR (400 MHZ, DMSO-D₆): δ 12.7 (brs, 1H), 9.21 (brs, 1H), 8.27 (s, 1H), 7.41 (d, J=7.2 Hz, 1H), 7.22 (s, 1H), 5.66 (d, J=7.2 Hz, 1H), 2.58 (d, J=7.2 Hz, 2H), 2.15 (s, 3H), 2.06 (sept., J=6.8 Hz, 1H), 0.89 (d, J=6.8 Hz, 6H) ppm.

### Example 14: Synthesis of tert-butyl (6-isobutyl-4-methylpyridin-3-yl)carbamate (CMPD-32)

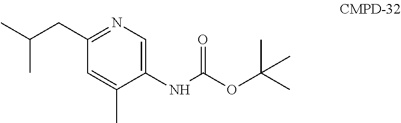 [SMILES: Cc1cc(CC(C)C)ncc1NC(=O)OC(C)(C)C]

In a reactor, a solution of 6-isobutyl-4-methylpyridin-3-amine (6.9 wt %, 2.9 kg, 1.218 mol) in toluene was concentrated to 2.0 L (10 wt % solution). A solution of ammonium chloride (33.3 wt %, 391.2 g, 2.435 mol) in water was charged at 25° C. Di-tert-butyl dicarbonate (399 g, 1.857 mol) was charged and the reaction mixture was warmed up to 50° C. After the reaction completed, the reaction mixture was cooled down to 25° C., water (200 mL) was added and the layers were separated. The organic layer was washed with water (1.0 L). The organic layer was concentrated to 0.5 L by distillation and hepatne (4.0 L) was charged at 50° C., then the reaction mixture was cooled down to 0° C. to crystallize the desired product (277 g, yield 86.2%). ¹H NMR (400 MHZ, DMSO-D₆): δ 8.66 (brs, 1H), 8.30 (s, 1H), 7.03 (s, 1H), 4.11 (q, J=6.8 Hz, 2H), 2.50 (d, J=7.2 Hz, 3H), 2.17 (s, 3H), 2.00 (m, 1H), 1.45 (s, 9H), 0.86 (d, J=6.4 Hz, 6H).

### Example 15: Synthesis of ethyl (6-isobutyl-4-methylpyridin-3-yl)carbamate (CMPD-33)

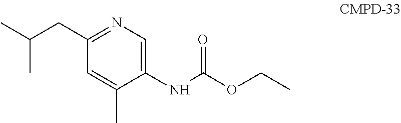 [SMILES: CCOC(=O)Nc1cnc(CC(C)C)cc1C]

In a reactor, 6-isobutyl-4-methylpyridin-3-amine (100 g, 609 mmol) in solution in tetrahydrofuran (150 mL) was added slowly (reaction is exothermic) to a mixture of tetrahydrofuran (200 mL), potassium tert-butoxide (103 g, 909 mmol), and diethyl carbonate (111 mL, 914 mmol) at 50° C. After the reaction completed, hepatne (200 mL) was charged at 50° C., then the reaction mixture was cooled down to 20° C. Water (150 mL) was added slowly (reaction is exothermic) and the layers were separated. The organic layer was washed with aqueous sodium chloide (23.5 wt %, 150 mL). This solution was solvent switched to heptane and cooled down to 5° C. to crystallize the desired product (134 g, yield 93.0%). ¹H NMR (400 MHz, DMSO-D₆): δ 8.93 (brs, 1H), 8.34 (s, 1H), 7.04 (s, 1H), 4.11 (q, J=6.8 Hz, 2H), 2.51 (d, J=7.2 Hz, 2H), 2.18 (s, 3H), 2.02 (m, 1H), 1.23 (t, J=6.8 Hz, 3H), 0.83 (d, J=6.8 Hz, 6H).

### Example 16: Synthesis of methyl (6-isobutyl-4-methylpyridin-3-yl)carbamate (CMPD-34)

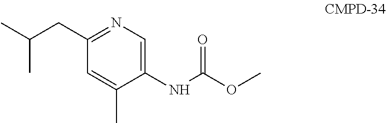 [SMILES: COC(=O)Nc1cnc(CC(C)C)cc1C]

The title compound was prepared in an analoguous manner of Example 15 using 6-isobutyl-4-methylpyridin-3-amine (60 g, 365 mmol), potassium tert-butoxide (82.8 g, 731 mmol) and dimethyl carbonate (52.5 mL, 621 mmol) instead of diethyl carbonate to obtain the desired product (76.0 g, yield 93.6%). ¹H NMR (400 MHZ, DMSO-D₆): δ 8.96 (brs, 1H), 8.34 (s, 1H), 7.05 (s, 1H), 3.65 (s, 3H), 2.51 (d, J=7.2 Hz, 2H), 2.18 (s, 3H), 2.02 (m, 1H), 0.86 (d, J=6.8 Hz, 6H).

### Example 17: Synthesis of sodium 5-(6-isobutyl-4-methylpyridin-3-yl)-2-(methoxycarbonyl)-4-oxo-4,5-dihydro-1-thia-3,5,8-triazaacenaphthylen-3-ide (CMPD-35)

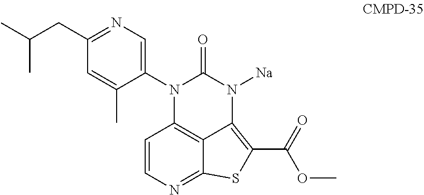

In a reactor, tert-butyl (6-isobutyl-4-methylpyridin-3-yl)carbamate (75 g, 283.7 mmol), 2,4-dichloro-3-iodopyridine (51.5 g, 297.8 mmol) and tetrabutylammonium bromide (9.2 g, 28.4 mmol) were added to toluene (450 mL) at 25° C. Water (37.5 mL) and 50 wt % sodium hydroxide (37.5 mL, 710 mmol) were added and the reaction mixture was stirred at 25° C. After the reaction completed, water (90 mL), and methyl thioglycolate (51 mL, 569.4 mmol) were added to the reaction mixture. After the reaction completed, the layers were separated. Methanol (225 mL) was added to the organic layer and the reaction mixture was warmed up to 50° C. 50 wt % sodium hydroxide (22.5 mL, 425.9 mmol) was slowly added and the reaction mixture was stirred at 50° C. After the reaction completed, the reaction mixture was cooled down to 20° C. and filtered to isolate the desired product cooled down to 20° C. to crystallize the desired product (112 g, yield 85.0%). ¹H NMR (400 MHZ, DMSO-D₆): δ 8.20 (s, 1H), 8.08 (d, J=5.6 Hz, 1H), 7.27 (s, 1H), 5.62 (d, J=5.6 Hz, 1H), 3.70 (s, 3H), 2.62 (d, J=7.2 Hz, 2H), 2.11 (m, 1H), 2.04 (s, 3H), 0.93 (d, J=6.0 Hz, 6H).

### Example 18: Synthesis of potassium 5-(6-isobutyl-4-methylpyridin-3-yl)-2-(methoxycarbonyl)-4-oxo-4,5-dihydro-1-thia-3,5,8-triazaacenaphthylen-3-ide (CMPD-36)

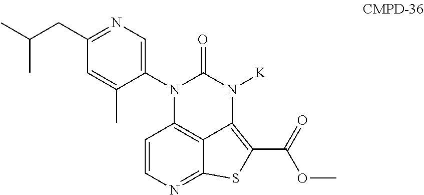

The title compound was prepared in an analoguous manner of Example 17 then methyl 5-(6-isobutyl-4-methylpyridin-3-yl)-4-oxo-4,5-dihydro-3H-1-thia-3,5,8-triazaacenaphthylene-2-carboxylate (15.0 g, 37.83 mmol) was dissolved in tetrahydrofuran (300 mL) at 20° C. and potassium tert-butoxide (4.28 g, 37.83 mmol) was slowly added to the reaction mixture and cooled down to 5° C. to crystallize the desired product (15.4 g, yield 80.8%). 1H NMR (400 MHZ, DMSO-D₆): δ 8.18 (s, 1H), 8.07 (d, J=5.6 Hz, 1H), 7.26 (s, 1H), 5.61 (d, J=5.6 Hz, 1H), 3.69 (s, 3H), 2.63 (d, J=6.8 Hz, 2H), 2.10 (m, 1H), 2.04 (s, 3H), 0.93 (d, J=6.8 Hz, 6H).

### Example 19: Crystallization-Induced Diastereomer Transformation (CIDT) of Formula (II)

Crystallization-Induced Diastereomer Transformation (CIDT) for a compound of Formula (II). The CIDT process is used to convert a mixture of P, M atropisomers (of any diastereomeric ratio, d.r. 100:0 to 0:100) of Formula (II) into the desired P-isomer, which is isolated as a dihydrate solid in the same process.

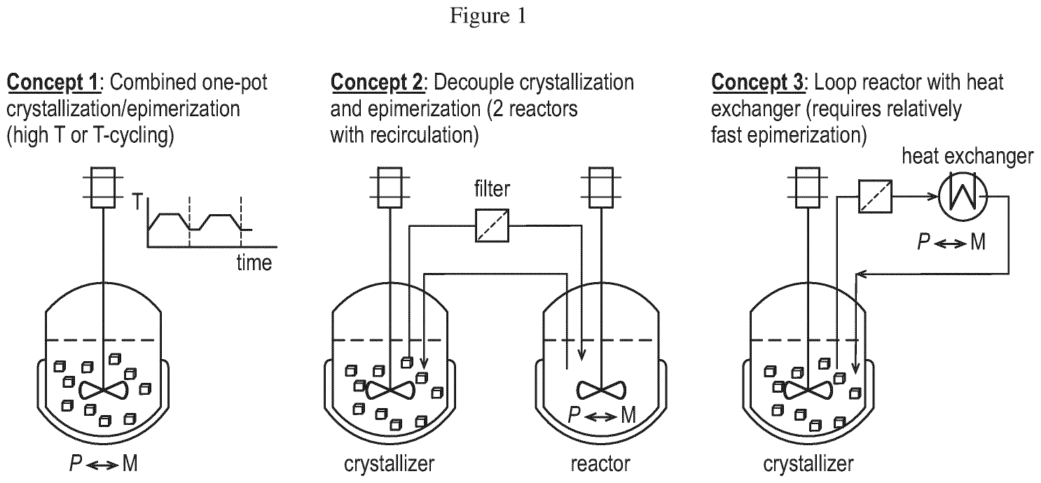

The CIDT process developed works by preferentially removing the P isomer out of a solution containing 1/1 P/M isomer ratio by crystallization, which in turn creates an enrichment in favour of the M isomer in the solution. Subsequently, an epimerization reaction converts the M isomer into the P isomer, which crystallizes further in the reactor. The process continues until most/all of the M isomer is converted to the P isomer. The preferential crystallization can take place in the same reactor as the epimerization, or most efficiently, the two processes can be spatially decoupled (by coupled stirred tanks or by stirred tanks coupled with tubular reactors in loop configuration), as shown in FIG. 1.

### Solubility and Epimierization

A broad solvent screen was performed in standard organic solvents (both water-miscible and immiscible solvents) to determine the epimerization kinetics and the main results are presented in Table 1 which shows rotational energy barrier, ΔG (KJ/mol) for epimerization of Formula (II), and half lives, t_{1/2} at 20° C., 85° C., 100° C. and 120° C. in various solvents and their water mixtures.

TABLE 1

| | | t_{1/2} at | t_{1/2} at | t_{1/2} at | t_{1/2} at |
|---|---|---|---|---|---|
| | ΔG | 20° C. | 85° C. | 100° C. | 120° C. |
| Solvent | (kJ/mol) | (years) | (min) | (min) | (min) |
| 3-methyl-1-butanol | 118.8 | 3 | 164 | 32 | 4 |
| 1-butanol | 119.2 | 3 | 189 | 36 | 5 |
| 2-butanol | 119.4 | 3 | 199 | 38 | 5 |
| 2-MeTHF | 119.4 | 3 | 203 | 39 | 5 |
| MIBK | 119.4 | 3 | 203 | 39 | 5 |
| EA | 119.6 | 4 | 217 | 41 | 6 |
| 3-methyl-1-butanol/water 91/9 | 119.7 | 4 | 224 | 43 | 6 |
| w/w % | | | | | |
| 1-butanol/water 85/15 w/w % | 120.0 | 4 | 248 | 47 | 6 |
| 1-propanol | 120.1 | 4 | 252 | 48 | 6 |
| IPAC | 120.2 | 5 | 260 | 49 | 7 |
| 2-butanol/water 70/30 w/w % | 120.4 | 5 | 285 | 54 | 7 |
| MEK | 120.5 | 5 | 291 | 55 | 7 |
| 1-propanol/water 80/20 w/w % | 120.6 | 6 | 301 | 57 | 7 |
| 1-propanol/water 67/33 w/w % | 120.7 | 6 | 312 | 59 | 8 |
| IPA | 120.8 | 6 | 324 | 61 | 8 |
| Propylene carbonate | 120.9 | 6 | 335 | 63 | 8 |
| propylene carbonate/water 93/7 | 121.1 | 7 | 360 | 67 | 9 |
| w/w % | | | | | |
| 1-propanol/water 50/50 w/w % | 121.2 | 7 | 369 | 69 | 9 |
| EtOH | 121.3 | 7 | 381 | 71 | 9 |
| 1-propanol/water 33/67 w/w % | 121.7 | 9 | 439 | 82 | 11 |
| Acetonitrile | 122.0 | 10 | 473 | 88 | 11 |
| Toluene | 125.6 | 43 | 1618 | 285 | 34 |

In parallel, an extensive solvent screen for solubility and form of P and M isomers showed that an important difference of solubility between P and M form exists in many solvent systems (Table 2, which shows the solubilities of P and M forms of Formula (II) at 10° C., 25° C. and 50° C. in various solvent systems and their water mixtures). In several solvent compositions (for example 1-propanol/water), the solubility difference is more than 10 fold. Moreover, this solubility difference can be tuned by changing the ratio between 1-propanol/water. Therefore, M form can be efficiently converted even starting from a 1:1 d.r. mixture.

TABLE 2

| | Solubility in mg/mL solution | | | | | |
|---|---|---|---|---|---|---|
| | 10° C. | | 25° C. | | 50° C. | |
| | P | M | P | M | P | M |
| Solvent | form | form | form | form | form | form |
| 1-Propanol | 206.8 | 500.4 | 197.2 | 265.4 | 255.0 | two |
| | | | | | | layers |
| 1-Propanol/water (20/80 w/w %) | 0.2 | 4.2 | 0.4 | 3.7 | 0.5 | 4.9 |
| 1-Propanol/water (33/67 w/w %) | 2.2 | two | 1.9 | 39.9 | 1.8 | 285.9 |
| | | layers | | | | |
| 1-Propanol/water (50/50 w/w %) | 8.1 | two | 8.5 | 187.5 | 15.6* | 276.3 |
| | | layers | | | | |
| 1-Propanol/water (67/33 w/w %) | 18.0 | 341.7 | 20.8 | 336.5 | 20.3* | 363.3 |
| 1-Propanol/water (80/20 w/w %) | 24.8 | 394.0 | 26.3 | 386.8 | 32.3* | 394.5 |
| Propylene carbonate | 55.8 | 163.1 | 50.1 | 73.7 | 97.3 | 107.0* |
| Propylene | 10.9 | 129.8 | 9.0 | 123.9 | 8.6 | 106.5 |
| carbonate/water (93/7 w/w %) | | | | | | |
| 1-Butanol | 137.3 | 397.5* | 132.8 | 266.2 | 197.6 | 444.7 |
| Isobutanol | 105.8 | 272.5* | 102.2 | 238.0 | 134.3 | 389.4 |
| 1-Butanol/water (~6/94 w/w %) | 0.01 | 0.2 | 0.01 | 0.3 | immiscible | immiscible |
| 1-Butanol/water (~80/20 w/w %) | 18.2 | 346.6 | 20.7 | 334.0 | immiscible | immiscible |
| Isobutanol/water (~8/92 w/w %) | 0.1 | 0.2 | 0.1 | 0.2 | 0.1 | 0.1 |
| 2-butanol/water (~17/83 w/w %) | 0.5 | 2.5 | 0.3 | 1.7 | 0.2 | 0.2 |
| Isopropanol/water (~16/84 w/w %) | 0.1 | 0.6 | 0.2 | 0.6 | 0.3 | 4.0 |
| Isopropanol/water (~28/72 w/w %) | 0.4 | 3.0 | 0.7 | 4.6 | 1.1 | 10.0 |
| Isopropanol/water (~44/56 w/w %) | 2.0 | 31.0 | 3.0 | 31.0 | 2.0 | >200 |
| Isopropanol/water (~61/39 w/w %) | 4.0 | 126.0 | 6.0 | 109.0 | 4.0 | >200 |

*form change was observed after 24 h equilibration.

### CIDT Protocol in 1-Propanol Water Solvent Mixture

### Phase 1: Crude Dissolution and Epimerization

During this phase, the crude P/M isomer mixture (of any d.r.) is dissolved in 1-propanol to give a homogeneous solution. Subsequently, the mixture is heated to an appropriate temperature and held for an appropriate amount of time to allow complete epimerization (P/M ratio of 1/1).

### Detailed Process Steps:

1) Charge 1 kg crude P or M isomer or mixture (purity crude used ≈92.2 wt % adjust values accordingly if different crude quality is used)
2) Add 2.49 L/kg 1-propanol
3) Start stirring at 300 rpm
4) Heat to 95° C. (all solids dissolve before reaching 95° C.)
5) Wait 10 h to fully epimerize
6) Cool to 25° C.
   Sample liq. to confirm complete epimerization→LC for diastereomeric ratio.

In case crude solution is used, perform solvent switch to 1-propanol. If crude is already a P/M mixture of 1/1 ratio, holding time of 10 h is not needed. Hence, holding time can be adapted to ensure complete dissolution (homogeneous mixture).

### Phase 2: Seeded Preferential Crystallization of P-Isomer

During this phase, supersaturation is generated in the P/M isomer mixture (1/1 ratio) by addition of water and seeds of the P-isomer are introduced to start preferential crystallization of that isomer. However, even in the absence of seeds, P-isomer will preferentially crystallize, and M will stay in solution.

### Detailed Process Steps:

1) Add 0.5 L/kg water
2) Seed with 0.01 kg/kg pure P-isomer (dihydrate)
3) Wait 5 h (seed ageing)
4) Add 1.5 L/kg water in 3 h
5) Cool to 10° C. at 0.2° C./min
6) Wait overnight to fully desaturate
   Sample liq. to confirm preferential P-isomer crystallization→LC for P and M concentration.

### Phase 3: Coupled Crystallization Epimerization in Flow Reactor

During this phase, the enriched mother liquor in the M-isomer is continuously filtered from the suspension and pumped into a tubular reactor held at a higher temperature to allow conversion to the desired P-isomer. The converted mother liquor is then returned to the crystallizer to allow additional P isomer crystallization.

### Detailed Process Steps:

1) Start mother liquor re-circulation through heated tubular loop reactor
   Parameters:
      a. In-line filter (KIMBLE 2 um Ultra-Ware SS Inlet filter)
      b. Loop reactor jacket: T=120° C.
      c. Flow rate=0.055 L/kg min
      d. Volume heated tube part ≈0.776 L/kg
      e. Residence time ≈14 min
2) Keep recirculating for 30 h
3) Add 2.66 L/kg water in 10 h while re-circulating (increase stirring to 550 rpm for EMAX 102 scale halfway to water dosing)
4) Wait 24 h while re-circulating
5) Return all liquid to crystallizer and stop re-circulation
   Sample liq. in time to track process→LC for P and M concentration.

### Phase 4: Product Isolation

During this phase, product is isolated from the suspension by filtration, washing is performed, followed by humidified drying.

### Detailed Process Steps:

1) Filter out the solids
2) Wash solids twice each time with 0.7 L/kg of 1-propanol/water 30/70 w/w %
3) Dry solids in vacuum at 40° C. under humidified conditions for 24 h
   Crystal Yield 86%/purity ≈(99/1 P/M).

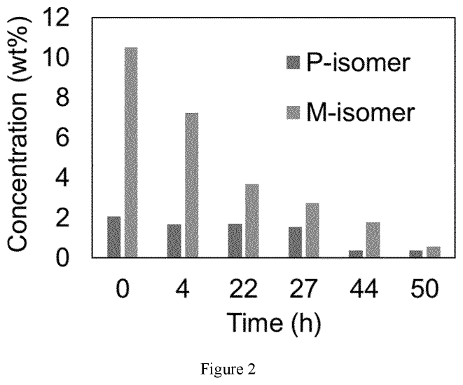

FIG. 2 shows typical evolution of P and M isomer concentrations in the liquid during re-circulation in the loop reactor in 1-propanol/water system.

### CIDT Protocol in Propylene Carbonate Water Solvent Mixture

### Phase 1: Crude Dissolution and Epimerization

During this phase, the crude P/M isomer mixture (of any d.r.) is dissolved in propylene carbonate to give a homogeneous solution. Subsequently, the mixture is heated to an appropriate temperature and held for an appropriate amount of time to allow complete epimerization (P/M ratio of 1/1).

### Detailed Process Steps:

1) Charge 1 kg crude P or M isomer or mixture (purity crude used ˜ 92.2 wt % adjust values accordingly if different crude quality is used)
2) Add 4.7 L/kg propylene carbonate
3) Start stirring at 350 rpm (on EMAX 102, adjust accordingly per setup/scale)
4) Heat to 100° C. (all solids dissolve before reaching 100° C.)
5) Wait 6 h to fully epimerize
6) Cool to 80° C.
   Sample liq. to confirm complete epimerization→LC for diastereomeric ratio.

### Phase 2: Preferential Crystallization of P-Isomer

During this phase, supersaturation is generated in the P/M isomer mixture (1/1 ratio) by addition of water and P-isomer preferential crystallization occurs.

### Detailed Process Steps:

1) Add 0.42 L/kg water
2) Cool to 25° C. over 4 h
3) Wait 5 h
   Sample liq. to confirm preferential P-isomer crystallization→LC for P and M concentration.
   Phase 3: Coupled crystallization epimerization in flow reactor

During this phase, the enriched mother liquor in the M-isomer is continuously filtered from the suspension and pumped into a tubular reactor held at a higher temperature to allow conversion to the desired P-isomer. The converted mother liquor is then returned to the crystallizer to allow additional P isomer crystallization.

### Detailed Process Steps:

1) Start mother liquor re-circulation through heated tubular loop reactor
   Parameters for JNJ setup:
      a. In-line filter (KIMBLE 2 um Ultra-Ware SS Inlet filter)
      b. Loop reactor jacket: T=130° C.
      c. Flow rate=0.055 L/kg min
      d. Volume heated tube part ≈0.776 L/kg
      e. Residence time ≈14 min
2) Keep recirculating for 52 h
3) Return all liquid to crystallizer and stop re-circulation
   Sample liq. to track process→LC for P and M concentration both<1 wt % process can be stopped.

### Phase 4: Product Isolation

During this phase, product is isolated from the suspension by filtration, washing is performed, followed by humidified drying.

### Detailed Process Steps:

1) Filter out the solids
2) Wash solids twice each time with 0.75 L/kg of propylene carbonate/water 93/7 w/w %
3) Dry solids in vacuum at 45° C. under humidified conditions for 24 h
   Crystal yield ≈83%/purity ≈(99/1 P/M).

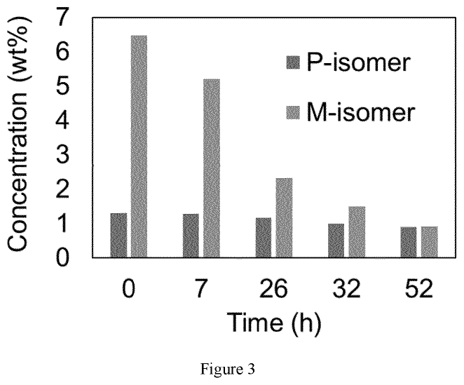

FIG. 3 shows typical evolution of P and M isomer concentrations in the liquid during re-circulation in the loop reactor in propylene carbonate/water system.

### Batch CIDT Protocol

In a flask, 5.0 g of 1:1 ratio P/M was added to a mixture of IPA/water 2/1 (25 mL). The mixture was heated to 80° C. for 1 hour then cooled down to 50° C. The mixture was stirred at 50° C. for 54 hours. The suspension was then cooled down to 10° C. The in-situ yield was 60% of P (and 40% of M). The suspension was filtered to yield P as a white solid (2.7 g, 88.9% assay, 48% yield).

### Example 20: NMR of N-((1R,2S)-2-acrylamidocyclopentyl)-5-(6-isobutyl-4-methylpyridin-3-yl)-4-oxo-4,5-dihydro-3H-1-thia-3,5,8-triazaacenaphthylene-2-carboxamide dihydrate (Formula P-(II))

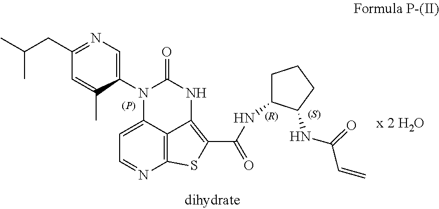

The following is a NMR spectra of material produced in Example 11. ¹H NMR (400 MHZ, DMSO-D₆): δ 10.1 (s, 1H), 8.46 (s, 1H), 8.33 (d, J=5.6 Hz, 1H), 7.88 (d, J=7.6 Hz, 1H), 7.77 (d, J=7.6 Hz, 1H), 7.36 (s, 1H), 6.22 (dd, J=6.8, 16.8 Hz, 1H), 6.11 (dd, J=2.4, 17.2 Hz, 1H), 5.94 (d, J=5.6 Hz, 1H), 5.57 (dd, J=2, 10 Hz, 1H), 4.32 (m, 2H), 2.65 (d, J=6.8 Hz, 2H), 2.13 (m, 4H), 2.10-1.4 (m, 6H), 0.93 (d, J=6.8 Hz, 1H).

### Example 21: SAR Studies

Initial structure-activity relationships for a new series of BTK inhibitors exemplified by Compound A (Table 3) have been previously conducted, (Tichenor, et al., ACS Med. Chem. Lett. 2021, 12, 782-790) with an acrylamide electrophile designed to engage in a covalent bond with Cys481. Despite the potent covalent inhibition potency in vitro and in vivo, the developability of Compound A was limited by the low aqueous solubility and low oral bioavailability (<1%) when dosed as a crystalline suspension, precluding its development as a therapeutic agent. Therefore, subsequent optimization of Compound A focused on increasing aqueous solubility and oral bioavailability from solid formulations suitable for preclinical tolerability assessment and advancement into human clinical trials. The development of the structure-activity relationship was conducted by characterizing the non-covalent affinity of each inhibitor for BTK (K_{I}) and the first order rate constant for irreversible covalent bond formation (k_{inact}). The second order rate constant k_{inact}/K_{I} ratio is used to assess the covalent inhibitor potency because it is independent of incubation time and substrate concentrations (Copeland, R. A. Evaluation of Enzyme Inhibitors in Drug Discovery: a Guide for Medicinal Chemists and Pharmacologists; 2nd ed.; Wiley: Hoboken, N.J., 2013).

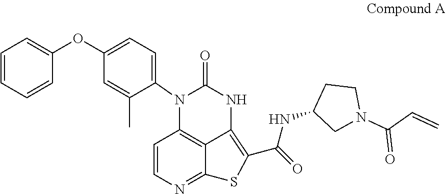 [SMILES: C=CC(=O)N1CC[C@@H](NC(=O)c2sc3nccc4c3c2NC(=O)N4c2ccc(Oc3ccccc3)cc2C)C1]

TABLE 3

| Parameter | Value^{a} |
|---|---|
| Kᵢ (μM) | 0.0288 ± 0.0210 |
| k_{inact} (s⁻¹) | 0.000346 ± 0.000025 |
| k_{inact}/K_{I} (M⁻¹s⁻¹) | 15,900 ± 11,000 |
| cLogP | 4.5 |
| pH 2/7 solubility (μM) | <4/<4 |
| Rat % F | 29.7% |
| (20% HP-β-CD solution) | |
| Rat % F (1 g/kg, crystalline | <1% |
| suspension) | |
| Liver microsomes T_{1/2} (min) | 23/40 |

^{a}Each k_{inact} and K_{I} value was obtained from an average of two independent determinations (±SD)

An initial set of analogs was designed to reduce lipophilicity by conducting a nitrogen scan on the proximal phenyl right (Table 4) while maintaining the tricyclic core and acrylamide. Compared to the highly potent phenyl derivative 2 (k_{inact}/K_{I}=13,832 s⁻¹), pyridyl isomers C and D imparted moderately increased aqueous solubility but a 20-fold loss in BTK potency. Incorporation of a methyl group ortho to the core heterocycle (5-10) restricts rotation around the N-aryl bond, yielding a pair of stable atropisomers (Tichenor, et al., ACS Med. Chem. Lett. 2021, 12, 782-790). Incorporation of an ortho-methyl into examples C and D increased BTK potency by improving the BTK k_{inact}/K_{I} ratio up to 16-fold (Compound F) by increasing the reversible binding affinity K_{I} (0.0276 μM) while the first order rate constant for covalent bond formation k_{inact} (0.000352±6e⁻⁰⁵ s⁻¹) remained unchanged across the series. The 4-methyl pyridyl Compound F maintained BTK potency that was comparable to Compound A with reduced cLogP, albeit with persistent low aqueous solubility and metabolic instability.

TABLE 4
Introduction of Back-Pocket Modifications to Increase Aqueous Solubility

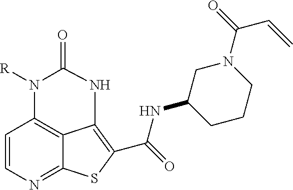

| | | | | | | Human liver | Solubility |
|---|---|---|---|---|---|---|---|
| | | | | k_{inact}/K_{I},^{a} | | microsomes | pH 2/7, |
| Compound | R = | k_{inact},^{a} s⁻¹ | K_{I},^{a} μM | M⁻¹s⁻¹ | cLogP | T_{1/2}, min | μM |
| B | 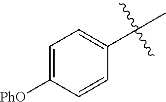 [SMILES: *c1ccc(Oc2ccccc2)cc1] | 0.000390 ± 0.00011 | 0.0320 ± 0.020 | 13800 ± 5200 | 4.3 | <4 | <4/<4 |
| C | 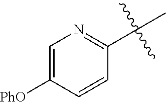 [SMILES: *c1ccc(Oc2ccccc2)cn1] | 0.000303 ± 2.5e⁻⁰⁵ | 0.456 ± 0.007 | 664 ± 44 | 3.8 | <4 | 10/9 |
| D | 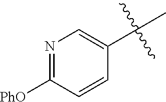 [SMILES: *c1ccc(Oc2ccccc2)nc1] | 0.000278 ± 0 | 0.354 ± 0.029 | 786 ± 63 | 3.4 | 6 | 18/4 |
| E^{b} | 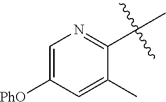 [SMILES: *c1ncc(Oc2ccccc2)cc1C] | 0.000333 ± 5.3e⁻⁰⁵ | 0.0699 ± 0.0002 | 4770 ± 7450 | 4.2 | 4 | 16/9 |
| F^{b} | 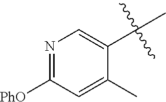 [SMILES: *c1cnc(Oc2ccccc2)cc1C] | 0.000352 ± 6e⁻⁰⁵ | 0.0276 ± 0.0083 | 13000 ± 1700 | 3.9 | 5 | 15/7 |
| G^{b} | 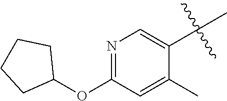 [SMILES: *c1cnc(OC2CCCC2)cc1C] | 0.000307 ± 3.6e⁻⁰⁵ | 0.0222 ± 0.0033 | 13800 ± 430 | 3.9 | <4 | 15/<4 |
| H^{b} | 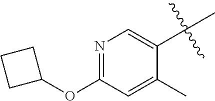 [SMILES: *c1cnc(OC2CCC2)cc1C] | 0.000316 ± 3.4e⁻⁰⁵ | 0.064 ± 0.014 | 5000 ± 560 | 3.4 | <4 | 10/4 |
| I^{b} | 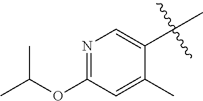 [SMILES: *c1cnc(OC(C)C)cc1C] | 0.000345 ± 1.2e⁻⁰⁵ | 0.208 ± 0.015 | 1660 ± 62 | 3.3 | 19 | 119/68 |
| J^{b} | 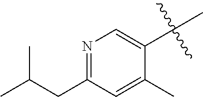 [SMILES: *c1cnc(CC(C)C)cc1C] | 0.000319 ± 5e⁻⁰⁶ | 0.145 ± 0.020 | 2225 ± 272 | 3.6 | 6 | 350/23 |

^{a}Each k_{inact} and K_{I} value was obtained from an average of 2 independent determinations (± SD).
^{b}Data corresponds to the more potent atropisomer

Subsequent analogs focused on replacing the distal aromatic ring with lower molecular weight aliphatic substituents to reduce lipophilicity and increase the fraction of sp³ hybridized carbon atoms (Compound B, Fsp³=13%). The cyclopentyloxy substituent in Compound G displayed equivalent potency compared to the phenoxy derivative F, but as the alkoxy substituent was further truncated with iteratively smaller alkoxy groups (examples H-J) with a corresponding reduction in cLogP, the corresponding BTK k_{inact}/K_{I} values declined up to 8-fold for Compound I (1663 M⁻¹s⁻¹) compared to Compound B with a concomitant improvement in microsomal stability and pH 2 and 7 aqueous solubility. Replacing the oxygen connection with a carbon as in isobutyl derivative J further improved pH 2 and 7 aqueous solubility (350/23 μM), with a moderate improvement in potency relative to Compound I. The improved profile of Compound I corresponded to increasing Fsp³ (32%) and reducing cLogP (3.6) relative to Compound B. Consistent with the SAR trends in Table 4, the non-covalent binding affinity component K_{I} had the largest impact on differences in inhibitor potency, while the k_{inact} values varied by less than 2-fold across a structurally diverse set of examples.

Further optimization of J was approached with the goal of identifying the optimal linker length to position the acrylamide to bind and engage Cys481 in covalent bond formation (Table 5). The BTK K_{I} proved to be sensitive to the length and orientation of the linker. Replacing the amino-piperidine in J with an amino-pyrrolidine (K) or an ethylene diamine (L) caused a reduction in potency by approximately 20-fold by reducing K_{I} to micromolar levels (2.55, 2.43, respectively). However, the improved microsomal stability of K and L relative to J justified additional linker exploration. The linker based on cis-cyclopentyl diamine (M) imparted considerably improved potency compared to other linkers (10, 11, 12) and increased microsomal stability compared to the piperidine derivative J (38 min vs 6 min). Compound M also maintained high pH 2 solubility, and measurable pH 7 solubility, making it an attractive candidate for in vivo profiling.

TABLE 5
Optimization of Diamine Acrylamide Linkers

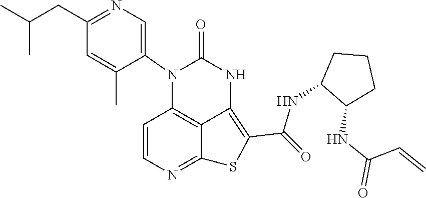 [SMILES: C=CC(=O)N[C@H]1CCC[C@H]1NC(=O)c1sc2nccc3c2c1NC(=O)N3c1cnc(CC(C)C)cc1C]

| | | | | | | Human liver | Solubility |
|---|---|---|---|---|---|---|---|
| | | | | k_{inact}/K_{I},^{a} | | microsomes | pH 2/7, |
| Compound | R = | k_{inact},^{a} s⁻¹ | K_{I},^{a} μM | M⁻¹s⁻¹ | cLogP | T_{1/2}, min | μM |
| K^{b} | 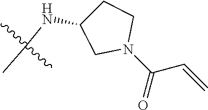 [SMILES: *N[C@@H]1CCN(C(=O)C=C)C1] | 0.000255 ± 1.0e⁻⁰⁵ | 2.55 ± 0.21 | 98.5 ± 12 | 3.1 | 53 | >400/172 |
| L^{b} | 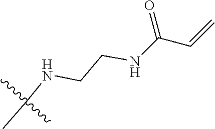 [SMILES: *NCCNC(=O)C=C] | 0.000317 ± 5.5e⁻⁰⁵ | 2.43 ± 0.45 | 131 ± 2 | 2.7 | 27 | >400/220 |
| M^{b} | 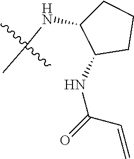 [SMILES: *N[C@@H]1CCC[C@@H]1NC(=O)C=C] | 0.000348 ± 1.9e⁻⁰⁵ | 0.0485 ± 0.0029 | 7290 ± 590 | 3.5 | 38 | 320/7 |

^{a}Each k_{inact} and K_{I} value was obtained from an average of 2 independent determinations ( ± SD).
^{b}Data corresponds to the more potent atropisomer

Relative to the series benchmark Compound A, Compound M is characterized by reduced lipophilicity and increased Fsp³ (32%), corresponding to increased pH 2 and 7 aqueous solubility while maintaining equivalent potency at suppressing anti-IgM induced B cell activation in human whole blood (Table 6). The activity of Compound M was assessed in rat and human whole blood assays using anti-IgD and anti-IgM stimulation, respectively, to trigger B-cell activation via B-cell receptor. While Compound M displays reduced potency in the mouse CD69 splenocyte assay, Compound M is 3-fold more potent in rat whole blood and equipotent in human whole blood potency compared to Compound A. The potent rat whole blood activity of Compound M may be attributed to the 6 to 10-fold increase in free fraction.

TABLE 6
Profile of Compound M (compound of Formula II)

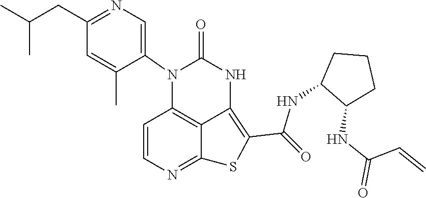 [SMILES: C=CC(=O)N[C@H]1CCC[C@H]1NC(=O)c1sc2nccc3c2c1NC(=O)N3c1cnc(CC(C)C)cc1C]

| Parameter | Ex. 13 | Ex. 1 |
|---|---|---|
| pH 2/7 solubility, μM | 320/7 | <4/<4 |
| Mouse CD69 cell, μM | 0.099 ± 0.068 | 0.011 ± 0041 |
| Rat whole blood IC₅₀, μM | 0.041 ± 0.012 | 0.13 ± 0.011 |
| Human whole blood IC₅₀, μM | 0.089 ± 0.012 | 0.084 ± 0.039 |
| PPB % free (Hu/rat/dog/cyno) | 3.1/3.7/6.3/3.2 | 0.16/0.47/0.12/0.25 |

### Example 22: XRPD Analysis of Crystalline Form B of Formula P-(II) Dihydrate

X-ray powder diffraction (XRPD) test was carried out on a Bruker D8 Advance X-ray powder diffractometer. The compound was spread on a mono-crystalline silicon plate and using weighing paper and a slight pressure to obtain a flat and homogeneous surface before testing. Details of the XRPD method used in the tests are shown in Table 7.

TABLE 7
XRPD Parameters

| Tube (s) | Cu/K-Alpha1 (λ = 1.54056 Å) |
|---|---|
| Voltage (KV) | 40 |
| Current (mA) | 40 |

Primary Beam path Radius 280

| | |
|---|---|
| TubeMount | Tube |
| Twin_Primary | Motorized Slit |
| SlitMount | No Slit 10.5 [mm] |
| SollerMount (deg) | Axial Soller 2.5 |

Secondary Beam path Radius 280

| | |
|---|---|
| LYNXEYE_XE_T | LYNXEYE_XE_T(1D mode) |
| Detector OpticsMount 2 (deg) | Soller_25 2.5 |
| Detector OpticsMount1 | Slit_open_1 18[mm] |
| SollerMount (deg) | Deflection 0 |
| SlitMount | No Slit 10.5 [mm] |
| Twin_Secondary | Motorized Slit |
| Sample stage | 1 |
| AUTOCHANGER | AirScatter |

Scan parameters

| | |
|---|---|
| Scan type | Coupled TwoTheta/Theta |
| Scan mode | Continuous PSD fast |
| Rotation | 60 r/min |
| 2 Theta (deg) | 3-50 |

The XRPD spectra is shown in FIG. 4 and the with significant peaks, d-spacing, and their intensities shown in Table 8.

TABLE 8
XRPD Peaks

| | | Net | Gross | Rel. | C. | |
|---|---|---|---|---|---|---|
| Angle | d Value | Intensity | Intensity | Intensity | Size | h, k, l |
| 5.599° | 15.77075 Å | 3344.64 | 3462.63 | 100.0% | | n.a. |
| 10.770° | 8.20788 Å | 83.5413 | 224.146 | 2.5% | | n.a. |
| 11.135° | 7.93955 Å | 1552.24 | 1702.19 | 46.4% | | n.a. |
| 12.134° | 7.28835 Å | 1185.29 | 1350.81 | 35.4% | | n.a. |
| 13.637° | 6.48800 Å | 768.502 | 934.228 | 23.0% | | n.a. |
| 15.515° | 5.70690 Å | 257.128 | 415.515 | 7.7% | | n.a. |
| 16.751° | 5.28831 Å | 307.000 | 465.275 | 9.2% | | n.a. |
| 17.115° | 5.17655 Å | 285.907 | 449.158 | 8.5% | | n.a. |
| 17.640° | 5.02383 Å | 602.749 | 769.733 | 18.0% | | n.a. |
| 18.012° | 4.92085 Å | 165.992 | 333.173 | 5.0% | | n.a. |
| 19.065° | 4.65126 Å | 1017.82 | 1183.58 | 30.4% | | n.a. |
| 19.929° | 4.45156 Å | 335.459 | 509.840 | 10.0% | | n.a. |
| 20.426° | 4.34442 Å | 2733.66 | 2914.14 | 81.7% | | n.a. |
| 21.013° | 4.22436 Å | 609.635 | 792.650 | 18.2% | | n.a. |
| 21.981° | 4.04053 Å | 490.786 | 681.079 | 14.7% | | n.a. |
| 22.328° | 3.97840 Å | 628.355 | 821.098 | 18.8% | | n.a. |
| 23.187° | 3.83294 Å | 1177.32 | 1372.43 | 35.2% | | n.a. |
| 24.017° | 3.70231 Å | 324.206 | 521.006 | 9.7% | | n.a. |
| 24.665° | 3.60659 Å | 1706.96 | 1904.61 | 51.0% | | n.a. |
| 26.058° | 3.41680 Å | 356.474 | 544.423 | 10.7% | | n.a. |
| 26.373° | 3.37670 Å | 1523.38 | 1708.15 | 45.5% | | n.a. |
| 27.538° | 3.23641 Å | 525.178 | 706.431 | 15.7% | | n.a. |
| 28.252° | 3.15627 Å | 541.798 | 730.277 | 16.2% | | n.a. |
| 28.887° | 3.08826 Å | 237.005 | 425.617 | 7.1% | | n.a. |
| 29.223° | 3.05354 Å | 249.312 | 435.598 | 7.5% | | n.a. |
| 29.824° | 2.99342 Å | 293.183 | 474.249 | 8.8% | | n.a. |
| 30.316° | 2.94595 Å | 907.731 | 1082.81 | 27.1% | | n.a. |
| 31.139° | 2.86994 Å | 115.840 | 275.285 | 3.5% | | n.a. |
| 32.514° | 2.75163 Å | 352.999 | 505.336 | 10.6% | | n.a. |
| 33.363° | 2.68347 Å | 38.1570 | 207.750 | 1.1% | | n.a. |
| 33.777° | 2.65156 Å | 88.1453 | 262.297 | 2.6% | | n.a. |
| 34.057° | 2.63040 Å | 276.376 | 452.189 | 8.3% | | n.a. |
| 34.575° | 2.59214 Å | 234.396 | 410.236 | 7.0% | | n.a. |
| 35.545° | 2.52361 Å | 397.811 | 575.093 | 11.9% | | n.a. |
| 36.338° | 2.47035 Å | 89.3288 | 273.685 | 2.7% | | n.a. |
| 36.692° | 2.44731 Å | 80.1799 | 267.389 | 2.4% | | n.a. |
| 37.161° | 2.41752 Å | 174.623 | 362.774 | 5.2% | | n.a. |
| 37.964° | 2.36817 Å | 228.818 | 411.070 | 6.8% | | n.a. |
| 38.228° | 2.35242 Å | 97.3911 | 275.630 | 2.9% | | n.a. |
| 39.707° | 2.26816 Å | 219.149 | 380.761 | 6.6% | | n.a. |
| 40.749° | 2.21254 Å | 69.2190 | 243.806 | 2.1% | | n.a. |
| 41.497° | 2.17436 Å | 127.204 | 318.675 | 3.8% | | n.a. |
| 41.982° | 2.15037 Å | 73.9256 | 271.939 | 2.2% | | n.a. |
| 42.218° | 2.13887 Å | 187.440 | 387.389 | 5.6% | | n.a. |
| 42.946° | 2.10428 Å | 121.873 | 322.626 | 3.6% | | n.a. |
| 44.173° | 2.04866 Å | 88.0773 | 288.021 | 2.6% | | n.a. |
| 44.771° | 2.02264 Å | 101.915 | 303.899 | 3.0% | | n.a. |
| 45.529° | 1.99073 Å | 58.4160 | 259.184 | 1.7% | | n.a. |
| 47.032° | 1.93053 Å | 67.9829 | 265.831 | 2.0% | | n.a. |
| 47.261° | 1.92173 Å | 111.501 | 312.671 | 3.3% | | n.a. |
| 47.596° | 1.90897 Å | 57.4924 | 262.147 | 1.7% | | n.a. |
| 48.056° | 1.89178 Å | 193.417 | 400.161 | 5.8% | | n.a. |
| 49.792° | 1.82982 Å | 67.4505 | 286.327 | 2.0% | | n.a. |

### Example 23: IR Analysis of Crystalline Form B of Formula P-(II) Dihydrate

IR was carried out on Thermo Nicoletis5 FT-IR Spectrometer with iD7 ATR-Diamond accessory. The compound was placed on the center of the crystal surface, and then the pressure control ring was rotated until the sample was pressed firmly against the crystal surface, then analyzed using the method shown in Table 9. The IR spectrum is shown in FIG. 5.

TABLE 9
IR Parameters

| Instrument | Thermo Nicoletis5 FT-IR Spectrometer |
|---|---|
| Accessory | iD7 ATR-Diamond |
| Number of scans | 32 |
| Resolution | 4 |
| Sample compartment | Main |
| Detector | DTGS KBr |
| Beamsplitter | KBr |
| Source | IR |
| Accessory | ID7 |
| Window | Diamond |
| Recommended range | 4000-525 cm⁻¹ |
| Gain: 1 | 1.0 |
| Optical velocity | 0.4747 |
| Aperture | 100 |

### Example 24: DSC Analysis of Crystalline Form B of Formula P-(II) Dihydrate

### Tested by Standard Pan:

Differential scanning calorimetry was carried out on a TA Instruments discovery DSC-2500 differential scanning calorimeter equipped with a RCS cooling unit. Transfer ˜3.4 mg of the compound into a standard aluminum sample pan from TA instruments. Close the sample pan with the standard lid and record the DSC curve. DSC testing with standard pan parameters are shown in Table10.

TABLE 10
DSC testing with standard pan parameters

| | | |
|---|---|---|
| Initial temperature | 30° | C. |
| Heating rate | 10° | C./min |
| Final temperature | 280° | C. |
| Nitrogen flow | 50 | mL/min |

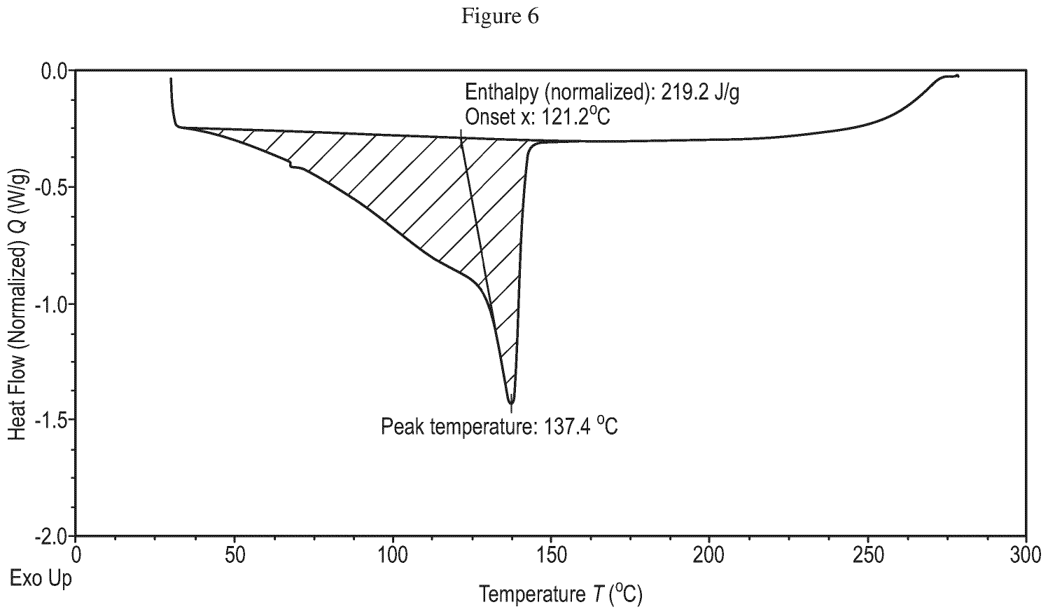

The DSC curve of Crystalline Form B of Formula P-(II) dihydrate tested by standard DSC pan shows one broad endothermic peak (middle point @ 137.4° C. with a heat fusion of 219 J/g), which is due to dehydration of water present in crystalline structure concomitant to the melting of the dehydrated structure (FIG. 6).

### Tested by Tzero Hermetically Sealed Pan:

Differential scanning calorimetry was carried out on a TA Instruments discovery DSC-2500 differential scanning calorimeter equipped with a RCS cooling unit. Transfer ˜1.6 mg of the compound into a Tzero aluminum sample pan from TA instruments. Close the sample pan with the Tzero lid (hermetically sealed) and record the DSC curve. DSC testing with Tzero hermetically sealed pan parameters are shown in Table11.

TABLE 11
DSC testing with by Tzero hermetically sealed pan parameters

| | | |
|---|---|---|
| Initial temperature | 30° | C. |
| Heating rate | 10° | C./min |
| Final temperature | 280° | C. |
| Nitrogen flow | 50 | mL/min |

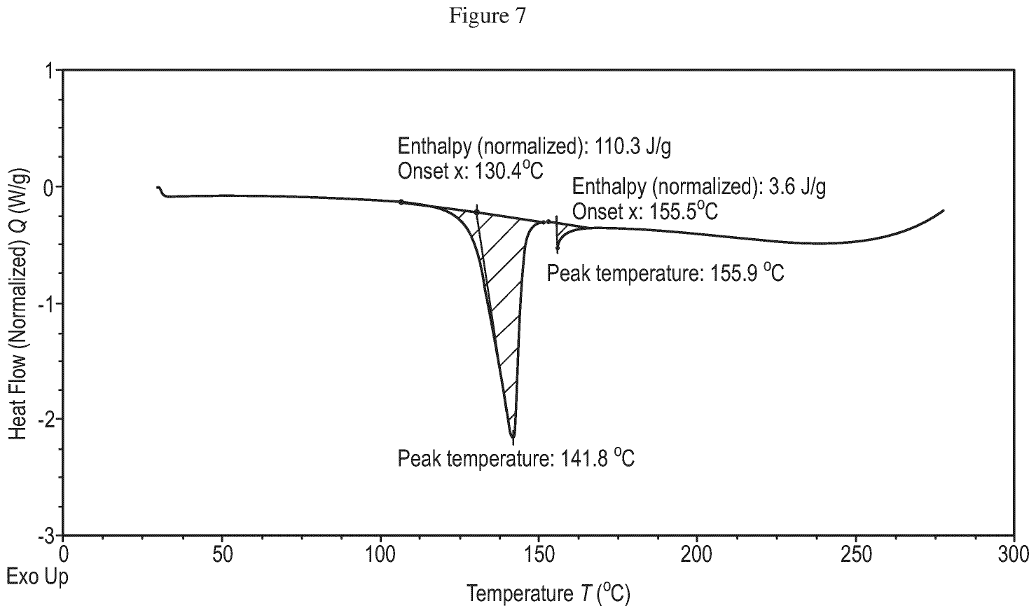

The DSC curve of Crystalline Form B of Formula P-(II) dihydrate tested by Tzero hermetically sealed pan shows two endothermic peaks, first endothermic peak should be the melting point of the compound at 141.8° C. (middle point) with a heat fusion of 110 J/g, the second endothermic peak occurs at 155.9° C. (middle point) with a heat fusion of 4 J/g (FIG. 7). The second endotherm cannot be assigned to a specific form under the specific measurement conditions.

### Example 25: XRPD Analysis of Crystalline Form B of Formula P-(II) Dihydrate

Thermogravimetric analysis (TGA) was carried out on a TA Instruments Q5000IR thermogravimetric analyzer. About 8.7 mg of the compound was transferred into a standard aluminum sample pan from TA instruments after it was tared and record the TGA curve. TGA testing parameters are shown in Table12.

TABLE 12
TGA Parameters

| | |
|---|---|
| Initial temperature | Room temperature |
| Heating rate | 20° C./min |
| Resolution factor | 4 (HiRes TGA) |
| Final condition | 350° C. or <80[(w/w)%] |

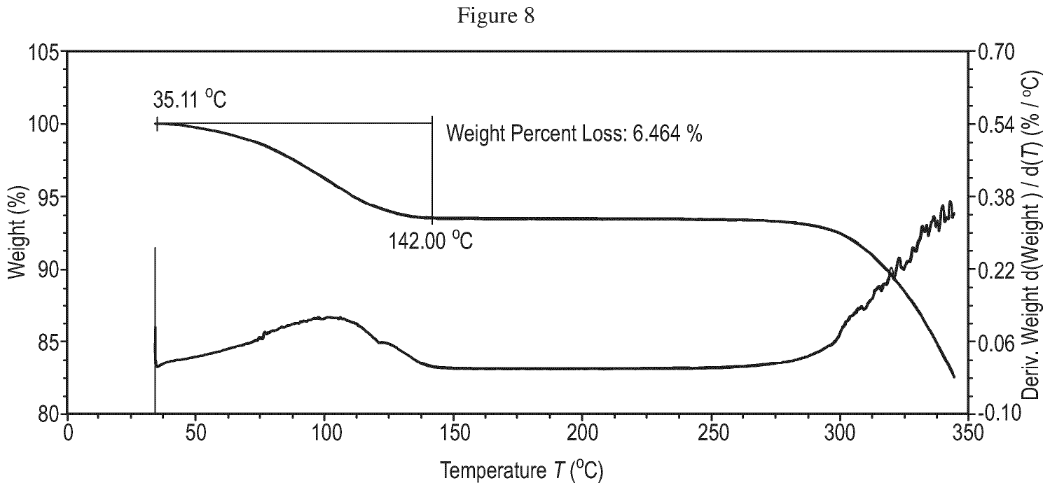

For Crystalline Form B of Formula P-(II) dihydrate, a weight loss of 6.5% (two equivalent of water) is registered in the temperature region from 35° C. to 142° C. (FIG. 8).

### Example 26: Rotational Barrier of Compound of Formula (II)

The rotational energy barrier of compound of Formula (II) atropisomers was estimated according to the method known in the art. The rotational barrier was determined in several solvents and their aqueous mixtures and the results are reported in Table 13, where an average value of 120.5 kJ mol⁻¹ or 28.8 kcal mol⁻¹, corresponding to epimerization half-lives of 63.8 and 4.0 months at 20° C. and 37° C., respectively, were obtained, placing this compound on the top segment of Class 2 atropisomers according to the classification systems known in the art. The rotational barrier is slightly influenced by the solvent and increases in the order 1-butanol<2-butanol<1-propanol<propylene carbonate. Additionally, increasing amounts of water increased substantially the rotational barrier in all solvents with the highest value obtained in 1-propanol/water 33/67 w/w % system. Unfortunately, due to the low aqueous solubility of compound of Formula (II) in water, epimerization studies could not be accurately conducted in pure water.

TABLE 13
Experimentally determined (80-100° C.) rotational energy barriers,
ΔGₑ^{‡}, and epimerization half-lives, t_{1/2} for compound of Formula
(II) in alcohols, propylene carbonate and various aqueous mixtures thereof.

| | ΔGₑ^{‡} | t_{1/2} at 20° C./37° C. |
|---|---|---|
| Solvent system | (kJ mol⁻¹)/(kcal mol⁻¹) | (months) |
| 1-butanol | 119.2/28.5 | 37.9/2.5 |
| 2-butanol | 119.4/28.5 | 40.1/2.6 |
| 1-butanol/water 85/15 w/w % | 120.0/28.7 | 52.6/3.3 |
| 1-propanol | 120.1/28.7 | 53.6/3.4 |
| 2-butanol/water 70/30 w/w % | 120.4/28.8 | 62.5/3.9 |
| 1-propanol/water 80/20 w/w % | 120.6/28.8 | 66.8/4.2 |
| 1-propanol/water 67/33 w/w % | 120.7/28.9 | 69.7/4.4 |
| propylene carbonate | 120.9/28.9 | 76.1/4.7 |
| propylene carbonate/water 93/7 w/w % | 121.1/29.0 | 83.2/5.2 |
| 1-propanol/water 50/50 w/w % | 121.2/29.0 | 85.6/5.3 |
| 1-propanol/water 33/67 w/w % | 121.7/29.1 | 105.9/6.5 |
| Average | 120.5 ± 0.77/28.8 ± 0.19 | |

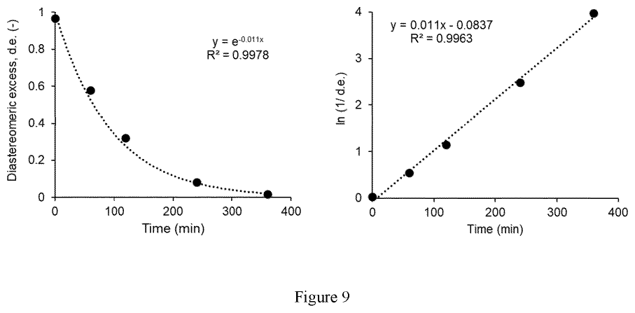

Enriched compound of Formula (II) of the P isomer was dissolved in the respective solvent to create a concentration of ˜1 wt % and was placed in a standard screw cap vial (1.8 mL) equipped with a magnetic stirring bar. Subsequently, the vial was placed in the heating block of a parallel reactor setup (Crystal16, Technobis B.V.) which was preheated to the desired temperature (80° C.-100° C., depending on the solvent). At given time intervals, small aliquot samples were taken in an HPLC vial, diluted, and the diastereomeric excess of the mixture was determined by an UPLC apparatus, equipped with an Acquity UPLC BEH C18 column (0.6 ml/min, gradient 10 mM ammonium acetate acetonitrile 95-5 v/v % and acetonitrile at 35° C.). The time points and corresponding decay of diastereomeric excess (d.e.) were plotted as t vs ln (1/d.e.) to determine the interconversion rate constant, rotational energy barrier and epimerization half-life at the respective solvent and temperature:

$$ln ( \frac{1 + [ P ] / [ M ]}{1 - [ P ] / [ M ]} ) = 2 {k}_{e} t + C = ln ( 1 / d . e . ) \qquad ( 1 )$$

$$Δ {G}_{e}^{‡} = - RT ln ( \frac{{k}_{e} h}{{k}_{B} T} ) \qquad ( 2 )$$

$${t}_{1 / 2} = ln 2 / 2 {k}_{e} \qquad ( 3 )$$

where [P], [M] are the concentrations of the P and M isomers, respectively, kₑ is the interconversion rate constant (s⁻¹), ΔGₑ^{‡} is the rotational energy barrier (KJ mol⁻¹), T is the absolute temperature, k_{B}, h and R are the Boltzmann, Planck and universal gas constants, respectively and t_{1/2} is the epimerization half-life. An example of the data treatment and determination procedure for kₑ is shown in FIG. 9 for propylene carbonate system at 100° C.

### Aspects

The disclosure is also directed to the following aspects:

Aspect 1. A method of synthesizing a compound of Formula (I):

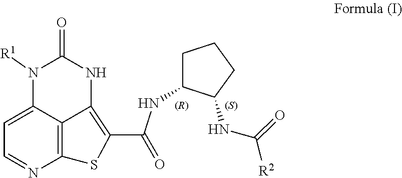

wherein:
   R¹ is selected from C₅₋₁₀ aryl and C₄₋₉ heteroaryl wherein the aryl or heteroaryl are optionally substituted with one or more groups independently selected from C₁₋₆ alkyl; and
   R² is selected from C₁₋₆ alkyl and C₂₋₆ alkenyl;
      comprising contacting the compound

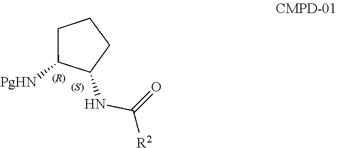

with an acid and a first solvent followed by contacting with the compound

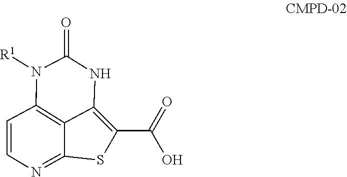

in the presence of one or more coupling reagents, an amine base, and a second solvent to obtain the compound of Formula (I).
Aspect 2. The method of aspect 1, wherein R¹ is C₄₋₉ heteroaryl optionally substituted with one or more groups independently selected from C₁₋₆ alkyl.
Aspect 3. The method of aspect 2, wherein R¹ is

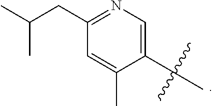 [SMILES: *c1cnc(CC(C)C)cc1C]

Aspect 4. The method of aspect 1, 2 or 3, wherein R² is C₂₋₆ alkenyl.
Aspect 5. The method of aspect 4, wherein the C₂₋₆ alkenyl is

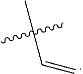 [SMILES: *C=C]

Aspect 6. The method of any of aspects 1-5, wherein the Pg is Boc.
Aspect 7. The method of any of aspects 1-6, wherein the acid is methanesulphonic acid.
Aspect 8. The method of any of aspects 1-7, wherein the first solvent is selected from dichloromethane (DCM), ethyl acetate (EtOAc), 2-methyltetrahydrofuran (MeTHF), tetrahydrofuran (THF), and acetonitrile (ACN).
Aspect 9. The method of aspect 8, wherein the first solvent is dichloromethane.
Aspect 10. The method of any of aspects 1-9, wherein the coupling reagent is selected from 1-Ethyl-3-(3-dimethylaminopropyl) carbodiimide (EDCI), hydroxybenzotriazole (HOBt), 1-[Bis(dimethylamino)methylene]-1H-1,2,3-triazolo[4,5-b]pyridinium 3-oxide hexafluorophosphate (HATU), propylphosphonic anhydride (T3P), 1,1′-carbonyldiimidazole (CDI), 2-hydroxypyridine-N-oxide (HOPO), and combinations thereof.
Aspect 11. The method of aspect 10, wherein the coupling reagent is 1-Ethyl-3-(3-dimethylaminopropyl) carbodiimide (EDCI), hydroxybenzotriazole (HOBt), or a combination thereof.
Aspect 12. The method of any of aspects 1-11, wherein the amine base is selected from N,N-diisopropylethylamine (DIPEA), triethylamine, tributylamine, N-methyl morpholine, and N-methyl piperidine.
Aspect 13. The method of aspects 12, wherein the amine base is N,N-diisopropylethylamine (DIPEA).
Aspect 14. The method of any of aspects 1-13, wherein the second solvent is is selected from dichloromethane (DCM), ethyl acetate (EtOAc), 2-methyltetrahydrofuran (MeTHF), tetrahydrofuran (THF), and acetonitrile (ACN).
Aspect 15. The method of aspect 14, wherein the second solvent is dichloromethane.
Aspect 16. The method of any of aspects 1-15, further comprising contacting the compound

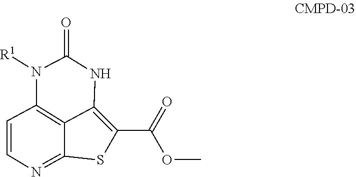

with a base and an ammonium salt in a solvent to form the compound CMPD-02.
Aspect 17. The method of aspect 16, wherein the base contacted with CMPD-03, is selected from potassium trimethylsilanolate (TMSOK), lithium hydroxide (LiOH), or sodium hydroxide (NaOH).
Aspect 18. The method of aspect 16 or 17, wherein the ammonium salt is selected from tetrabutylammonium bromide (TBAB), tetrabutylammonium hydrogen sulfate, benzyltrimethylammonium bromide, benzyltrimethylammonium chloride, and Aliquat 336.
Aspect 19. The method of aspect 18, wherein the ammonium salt is tetrabutylammonium bromide (TBAB).
Aspect 20. The method of any of aspects 16-19, wherein the solvent in which CMPD-03 is converted to CMPD-02, is tetrahydrofuran.
Aspect 21. The method of any of aspects 16 to 20, further comprising contacting the compound

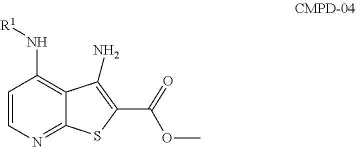

with a carbonyl source and a solvent to form the compound CMPD-03.
Aspect 22. The method of aspect 21, wherein the carbonyl source is selected from N,N′-carbonyldiimidazole (CDI), phosgene, triphosgene, and N,N′-disuccinimidyl carbonate (DSC).
Aspect 23. The method of aspect 22, wherein the carbonyl source is N,N′-carbonyldiimidazole (CDI).
Aspect 24. The method of aspect 21, 22 or 23, wherein the solvent in which CMPD-04 is converted to CMPD-03, is tetrahydrofuran.
Aspect 25. The method of any of aspects 21-24, further comprising contacting the compound

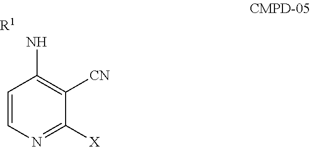

with a sulfur reagent and a base in a solvent to form the compound CMPD-04 and wherein X is selected from Cl and Br.
Aspect 26. The method of aspect 25, wherein X is Cl.
Aspect 27. The method of aspect 25 or 26, wherein the sulfur reagent is methyl 2-mercaptoacetate.
Aspect 28. The method of aspect 25, 26 or 27, wherein the base contacted with CMPD-05, is NaOMe.
Aspect 29. The method of aspect 25, 26, 27 or 28, wherein the solvent in which CMPD-05 is converted to CMPD-04, is methanol.
Aspect 30. The method of any of aspects 25, 26, 27, 28 or 29, further comprising contacting the compound

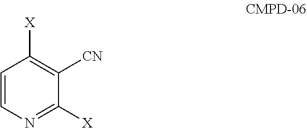

with the compound R¹—NH₂ with a base, and an amine base in a solvent system to form the compound CMPD-05.
Aspect 31. The method of aspect 30, wherein X is Cl.
Aspect 32. The method of aspect 30 or 31, wherein R¹ is

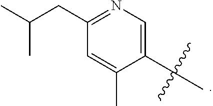 [SMILES: *c1cnc(CC(C)C)cc1C]

Aspect 33. The method of aspect 30, 31 or 32, wherein the base contacted with CMPD-06, is K₂CO₃.
Aspect 34. The method of aspect 30, 31, 32 or 33, wherein the amine base contacted with CMPD-06, is N,N-diisopropylethylamine (DIPEA).
Aspect 35. The method of aspect 30, 31, 32, 33 or 34, wherein the solvent system in which CMPD-06 is converted to CMPD-05, is toluene/NMP.
Aspect 36. The method of aspect 30, 31, 32, 33, 34 or 35, further comprising contacting the compound R¹—NO₂ with a hydrogen source, a catalyst, and a solvent system to form the compound R¹—NH₂.
Aspect 37. The method of aspect 36, wherein the hydrogen source is NaBH₄ or H₂.
Aspect 38. The method of aspect 36 or 37, wherein the catalyst is selected from NiCl₂, Pd/C, Pd(OAc)₂, Pd(OH)₂. Raney Ni, Sponge Ni, Pt/V/C, Pt/Fe/C, NiBr₂, NiCl₂ (DME).
Aspect 39. The method of aspect 38, wherein the catalyst is NiCl₂ or Pd/C.
Aspect 40. The method of aspect 36, 37, 38 or 39, wherein the solvent system in which R¹—NO₂ is converted to R¹—NH₂, is selected from toluene/water, xylene, THF, MeTHF, MeOH, THF/MeOH, Toluene/MeOH, MeTHF/water, and THF/water.
Aspect 41. The method of aspect 40, wherein the solvent system in which R¹—NO₂ is converted to R¹—NH₂, is toluene/water.
Aspect 42. The method of aspect 32, 33, 34, 35, 36, 37, 38, 39, 41 or 41, further comprising contacting the compound

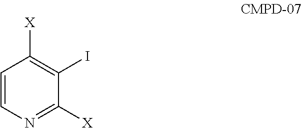

with a cyano source and a solvent to form the compound CMPD-06.
Aspect 43. The method of aspect 42, wherein X is Cl.
Aspect 44. The method of aspect 42 or 43, wherein the cyano source is CuCN.
Aspect 45. The method of aspect 42, 43 or 44, wherein the solvent in which CMPD-07 is converted to CMPD-06, is butyronitrile.
Aspect 46. The method of aspect 42, 43, 44 or 45, further comprising contacting the compound

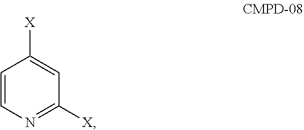

with a base and an electrophilic halogen in a solvent to form the compound CMPD-07.
Aspect 47. The method of aspect 46, wherein X is Cl.
Aspect 48. The method of aspect 46 or 47, wherein the base contacted with CMPD-08, is lithium diisopropylamine.
Aspect 49. The method of aspect 46, 47 or 48, wherein the solvent in which CMPD-08 is converted to CMPD-07, is tetrahydrofuran.
Aspect 50. The method of any of aspects 1-49, further comprising contacting the compound

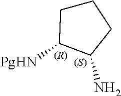

CMPD-09 with a base,

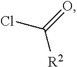

and a solvent to form the compound CMPD-01.
Aspect 51. The method of aspect 50, wherein R² is C₂₋₆ alkenyl.
Aspect 52. The method of aspect 51, wherein the C₂₋₆ alkenyl is

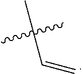 [SMILES: *C=C]

Aspect 53. The method of aspect 50, 51 or 52, wherein the Pg is Boc.
Aspect 54. The method of aspect 50, 51, 52 or 53, wherein the base contacted with CMPD-09, is NaHCO₃.
Aspect 55. The method of aspect 50, 51, 52, 53 or 54, wherein the solvent in which CMPD-09 is converted to CMPD-01, is 2-methyltetrahydrofuran.
Aspect 56. A method of synthesizing a compound of Formula (II):

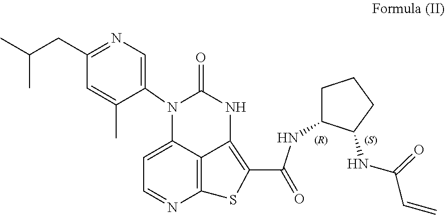

comprising contacting the compound

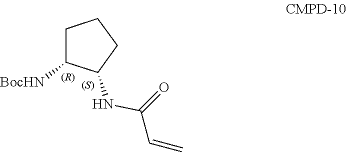

with an acid and a first solvent followed by contacting with the compound

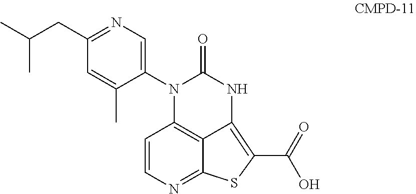 [SMILES: Cc1cc(CC(C)C)ncc1N1C(=O)Nc2c(C(=O)O)sc3nccc1c23]

in the presence of one or more coupling reagents, an amine base, and a second solvent to obtain the compound of Formula (II).
Aspect 57. The method of aspect 56, wherein the acid is methanesulphonic acid.
Aspect 58. The method of aspect 56 or 57, wherein the first solvent is dichloromethane.
Aspect 59. The method of aspect 56, 57 or 58, wherein the coupling reagent is 1-Ethyl-3-(3-dimethylaminopropyl) carbodiimide (EDCI).
Aspect 60. The method of aspect 56, 57, 58 or 59, wherein the amine base is N,N-diisopropylethylamine (DIPEA).
Aspect 61. The method of aspect 56, 57, 58, 59 or 60, wherein the second solvent is dichloromethane.
Aspect 62. The method of aspect 56, 57, 58, 59, 60 or 61, further comprising contacting the compound

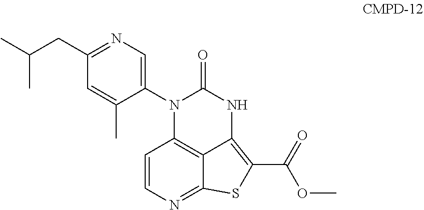 [SMILES: COC(=O)c1sc2nccc3c2c1NC(=O)N3c1cnc(CC(C)C)cc1C]

with a base and an ammonium salt in a solvent to form the compound CMPD-11.
Aspect 63. The method of aspect 62, wherein the base contacted with CMPD-12, is potassium trimethylsilanolate (TMSOK).
Aspect 64. The method of aspect 62 or 63, wherein the ammonium salt is tetrabutylammonium bromide (TBAB).
Aspect 65. The method of aspect 62, 63 or 64, wherein the solvent in which CMPD-12 is converted to CMPD-11, is tetrahydrofuran.
Aspect 66. The method of aspect 62, 63, 64 or 65, further comprising contacting the compound

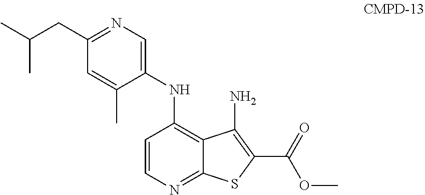 [SMILES: COC(=O)c1sc2nccc(Nc3cnc(CC(C)C)cc3C)c2c1N]

with a carbonyl source and a solvent to form the compound CMPD-12.
Aspect 67. The method of aspect 66, wherein the carbonyl source is N,N′-carbonyldiimidazole (CDI).
Aspect 68. The method of aspect 66 or 67, wherein the solvent in which CMPD-13 is converted to CMPD-12, is tetrahydrofuran.
Aspect 69. The method of aspect 66, 67 or 68, further comprising contacting the compound

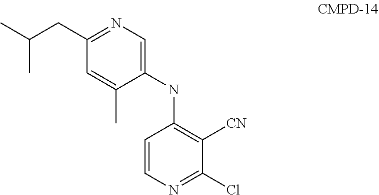 [SMILES: Cc1cc(CC(C)C)ncc1Nc1ccnc(Cl)c1C#N]

with a sulfur reagent and a base in a solvent to form the compound CMPD-13.
Aspect 70. The method of aspect 69, wherein the sulfur reagent is methyl 2-mercaptoacetate.
Aspect 71. The method of aspect 69 or 70, wherein the base contacted with CMPD-14, is NaOMe.
Aspect 72. The method of aspect 69, 70 or 71, wherein the solvent in which CMPD-14 is converted to CMPD-13, is methanol.
Aspect 73. The method of aspect 69, 70, 71 and 72, further comprising contacting the compound

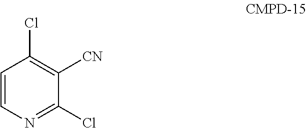

with the compound

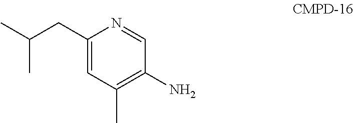

with a base, and an amine base in a solvent system to form the compound CMPD-14.
Aspect 74. The method of aspect 73, wherein the base contacted with CMPD-15 and CMPD-16, is K₂CO₃.
Aspect 75. The method of aspect 73 or 74, wherein the amine base contacted with CMPD-15 and CMPD-16, is N,N-diisopropylethylamine (DIPEA).
Aspect 76. The method of aspect 73, 74 or 75, wherein the solvent system in which CMPD-15 and CMPD-16 are converted to CMPD-14, is toluene/NMP.
Aspect 77. The method of aspect 73, 74, 75 or 76, further comprising contacting the compound

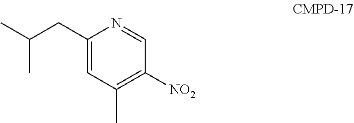

with a hydride source, a catalyst, and a solvent system to form the compound CMPD-16.
Aspect 78. The method of aspect 77, wherein the hydride source is NaBH₄.
Aspect 79. The method of aspect 77 or 78, wherein the catalyst is NiCl₂.
Aspect 80. The method of aspect 77, 78 or 79, wherein the solvent system in which CMPD-17 is converted to CMPD-14, is toluene/water.
Aspect 81. The method of aspect 77, 78, 79 or 80, further comprising contacting the compound

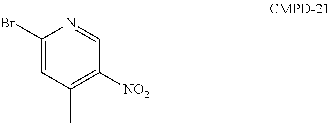

with a catalyst, phosphorus reagent, a boron reagent, a first base, a second base and a solvent system to form the compound CMPD-17.
Aspect 82. The method of aspect 81, wherein the catalyst contacted with CMPD-17 is Pd(OAc)₂.
Aspect 83. The method of aspect 81 or 82, wherein the phosphorus reagent is di(1-adamantyl)-n-butylphosphine.
Aspect 84. The method of aspect 81, 82 or 83, wherein the boron reagent is isobutylboronic acid.
Aspect 85. The method of aspect 81, 82, 83 or 84, wherein the first base is K₂CO₃.
Aspect 86. The method of aspect 81, 82, 83, 84 or 85, wherein the second base is KOAc.
Aspect 87. The method of aspect 81, 82, 83, 84, 85 or 86, wherein the solvent system in which CMPD-20 is converted to CMPD-17, is toluene/water.
Aspect 88. The method of any of aspects 81-87, further comprising contacting the compound

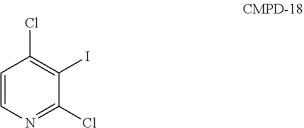

with a cyano source and a solvent to form the compound CMPD-15.
Aspect 89. The method of aspect 88, wherein the cyano source is CuCN.
Aspect 90. The method of aspect 88 or 89, wherein the solvent in which CMPD-18 is converted to CMPD-15, is butyronitrile.
Aspect 91. The method of aspect 88, 89 or 90, further comprising contacting the compound

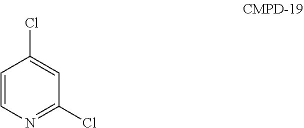

with a base and an electrophilic halogen in a solvent to form the compound CMPD-18.
Aspect 92. The method of aspect 91, wherein the base contacted with CMPD-19 is lithium diisopropylamine.
Aspect 93. The method of aspect 91 or 92, wherein the solvent in which CMPD-19 is converted to CMPD-18, is tetrahydrofuran.
Aspect 94. The method of aspect 91, 92 or 93, further comprising contacting the compound

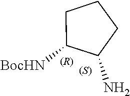

CMPD-20 with a base,

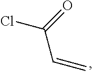

and a solvent to form the compound CMPD-10.
Aspect 95. The method of aspect 94, wherein the base contacted with CMPD-20 is NaHCO₃.
Aspect 96. The method of aspect 94 or 95, wherein the solvent in which CMPD-20 is converted to CMPD-10, is 2-methyltetrahydrofuran.
Aspect 97. A method of isolating the P atropisomer from a mixture of M and P isomers of compound of Formula (II), comprising the steps of:
1) Dissolve the compound of Formula (II) in one or more solvents in a vessel to form a solution,
2) Optionally, heat the solution to a first temperature of about 75° C. to about 140° C. for a first time period of about 8 hours to about 12 hours, and cool the solution to a second temperature of about 20° C. to about 30° C.,
3) Add water and wait for a second time period of about 2 hours to 24 hours to form a suspension. Optionally, seed with a compound of Formula P-(II) during this step. Optionally, cool the solution to a third temperature about 5° C. to about 15° C. for a third time period of about 6 hours to about 12 hours to form a suspension,
4) Filter the suspension to obtain the crystals of compound of Formula P-(II),
5) Collect the filtrate and heat it to a fourth temperature of about 75° C. to about 150° C. for a fourth time period of about about 5 minutes to about 1 hour,
6) Repeat steps 3-5 about 20-100 times until all the M isomer is converted to P isomer,
7) Collect the precipitate, wash, and dry the solid under humidified conditions.
   Aspect 98. The method of aspect 97, wherein the first one or more solvents is selected from the group consisting of isopropanol, propylene carbonate, water, 1-propanol, 1-butanol, 2-butanol, isobutanol, propylene glycol, ethylene glycol, dimethylacetamide (DMA), benzyl alcohol, N-methyl-2-pyrrolidone (NMP), propylene glycol methyl ether, 2-ethoxyethanol, (s)-1,2-Butanediol, transcutol, ethylene glycol diacetate, and combinations thereof.
   Aspect 99. The method of aspect 98, wherein the first one or more solvents is 1-propanol.
   Aspect 100. The method of aspect 97, 98 or 99, wherein the first temperature is about 95° C.
   Aspect 101. The method of aspect 97, 98, 99 or 100, wherein the first time period is about 10 hours.
   Aspect 102. The method of aspect 97, 98, 99, 100 or 101, wherein the second temperature is about 25° C.
   Aspect 103. The method of aspect 97, 98, 99, 100, 101 or 102, wherein the second time period is about 5 hours.
   Aspect 104. The method of aspect 97, 98, 99, 100, 101, 102 or 103, wherein the third temperature is about 10° C.
   Aspect 105. The method of aspect 97, 98, 99, 100, 101, 102, 103 or 104, wherein the third time period is about 8 hours.
   Aspect 106. A method of converting a compound of Formula (II) to compound of Formula P-(II) comprising the steps of:
1) Dissolving the compound of Formula (II) in a first one or more solvents in a first vessel to form a solution,
2) Heating the solution to a first temperature of about 75° C. to about 120° C. and hold at the first temperature for a first time period of about 4 hours to about 8 hours,
3) Cool the solution to a second temperature of about 60° C. to about 100° C.,
4) Add water and to solution a compound of Formula (II)
5) Cool the solution to third temperature about 10° C. to about 30° C. over a second time period of about 2 hours to about 8 hours and wait for a third time period of about 3 hours to about 9 hours to Form B suspention.
6) Transfer the mother liquor from the formed suspention to a second vessel,
7) Heat the mother liquor to a fourth temperature of about 110° C. to about 150° C. for a fourth time period of about 30 hours to about 70 hours,
8) Return the mother liquor to the first vessel,
9) Collect the formed crystals of the compound of Formula P-(II) by filtration,
10) Wash the crystals with a second one or more solvents, and
11) Dry the crystals of the compound of Formula P-(II) at a fifth temperature of about 30° C. to about 50° C. under humidified condtions for a fifth time period of about 12 hours to about 36 hours.
   Aspect 107. The method of aspect 106, wherein the first one or more solvents is selected from the group consisting of isopropanol, propylene carbonate, water, 1-propanol, 1-butanol, 2-butanol, isobutanol, propylene glycol, ethylene glycol, dimethylacetamide (DMA), benzyl alcohol, N-methyl-2-pyrrolidone (NMP), propylene glycol methyl ether, 2-ethoxyethanol, (s)-1,2-Butanediol, transcutol, ethylene glycol diacetate, and combinations thereof.
   Aspect 108. The method of aspect 107, wherein the first one or more solvents is propylene carbonate.
   Aspect 109. The method of aspect 106, 107 or 108, wherein the first temperature is about 100° C.
   Aspect 110. The method of aspect 106, 107, 108 or 109, wherein the first time period is about 6 hours.
   Aspect 111. The method of aspect 106, 107, 108, 109 or 110, wherein the second temperature is about 80° C.
   Aspect 112. The method of aspect 106, 107, 108, 109, 110 or 111, wherein the third temperature is about 25° C.
   Aspect 113. The method of aspect 106, 107, 108, 109, 110, 111 or 112, wherein the second time period is about 4 hours.
   Aspect 114. The method of aspect 106, 107, 108, 109, 110, 111, 112 or 113, wherein the third time period is about 5 hours.
   Aspect 115. The method of aspect 106, 107, 108, 109, 110, 111, 112, 113 or 114, wherein the fourth temperature is about 130° C.
   Aspect 116. The method of aspect 106, 107, 108, 109, 110, 111, 112, 113, 114 or 115, wherein the fourth time period is about 52 hours.
   Aspect 117. The method of any of aspects 106-116, wherein the second one or more solvents is selected from the group consisting of isopropanol, propylene carbonate, water, 1-propanol, 1-butanol, 2-butanol, isobutanol, propylene glycol, ethylene glycol, dimethylacetamide (DMA), benzyl alcohol, N-methyl-2-pyrrolidone (NMP), propylene glycol methyl ether, 2-ethoxyethanol, (s)-1,2-Butanediol, transcutol, ethylene glycol diacetate, and combinations thereof.
   Aspect 118. The method of aspect 117, wherein the second one or more solvents are propylene carbonate and water.
   Aspect 119. The method of any of aspects 106-118, wherein the fifth temperature is about 45° C.
   Aspect 120. The method of any of aspects 106-119, wherein the fifth time period is about 24 hours.
   Aspect 121. Crystalline Form B of Formula P-(II) dihydrate:

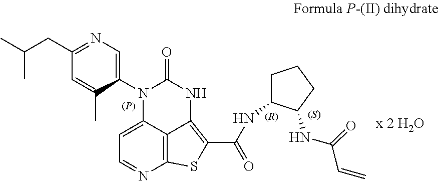

Aspect 122. The crystalline Form B of aspect 121, wherein crystalline Form B is characterized by a XRPD pattern having a peak expressed in degrees 2θ (±0.2) at about 5.599°.
Aspect 123. The crystalline Form B of aspect 122, wherein Form B is further characterized by a XRPD pattern having a peak expressed in degrees 2θ (±0.2) at about 20.426°.
Aspect 124. The crystalline Form B of aspect 123, wherein Form B is further characterized by a XRPD pattern having a peak expressed in degrees 2θ (±0.2) at about 24.665°.
Aspect 125. The crystalline Form B of aspect 124, wherein Form B is further characterized by a XRPD pattern having peaks expressed in degrees 2θ (±0.2) at about 11.135° and about 26.373°.
Aspect 126. The crystalline Form B of aspect 125, wherein Form B is further characterized by a XRPD pattern having peaks expressed in degrees 2θ (±0.2) at about 12.134°, about 23.187°, about 19.065°, and about 30.316°.
Aspect 127. The crystalline Form B of aspect 121, wherein Form B is characterized by a XRPD pattern substantially as shown in FIG. 4.
Aspect 128. The crystalline Form B of any of aspects 121-127, wherein crystalline Form B is characterized by an IR peak at about 1522 cm⁻¹.
Aspect 129. The crystalline Form B of aspect 128, wherein Form B is further characterized by an IR peak at about 1714 cm⁻¹.
Aspect 130. The crystalline Form B of aspect 129, wherein Form B is further characterized by an IR peak at about 1642 cm⁻¹ and about 1622 cm⁻¹.
Aspect 131. The crystalline Form B of aspect 130, wherein Form B is further characterized by an IR peak at about 1270 cm⁻¹ and about 1251 cm⁻¹.
Aspect 132. The crystalline Form B of aspect 131, wherein Form B is further characterized by an IR peak at about 1541 cm⁻¹ and about 1494 cm⁻¹.
Aspect 133. The crystalline Form B of aspect 121, wherein Form B is characterized by an IR pattern substantially as shown in FIG. 5.
Aspect 134. The crystalline Form B of aspect 121, wherein Form B is characterized by one or more of 1) a DSC thermograms utilizing standard pan conditions exhibiting an endotherm at about 196.8° C.; 2) a DSC thermograms utilizing Tzero hermetically sealed pan conditions exhibiting a first endotherm at about 141.8° C. and a second endotherm at about 155.9° C.; and 3) a water loss as measured by thermogravimetric analysis of about 6.5 wt. %.
Aspect 135. A compound having the structure:

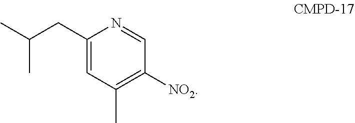

Aspect 136. A compound selected from

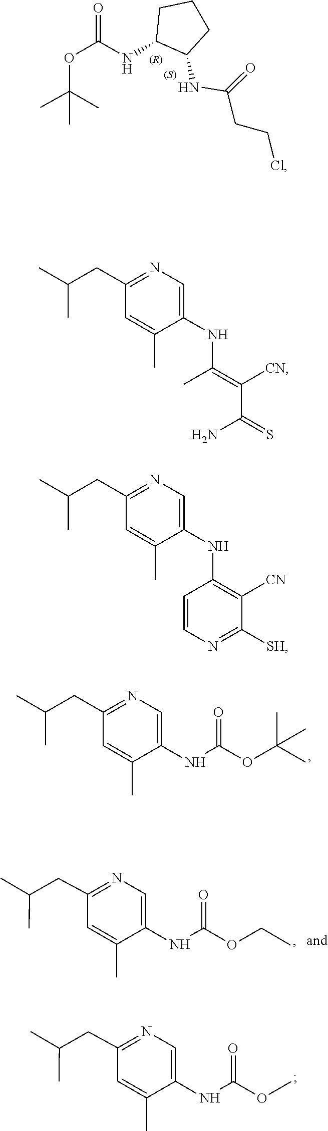

and pharmaceutically acceptable salts thereof.
Aspect 137. A compound selected from

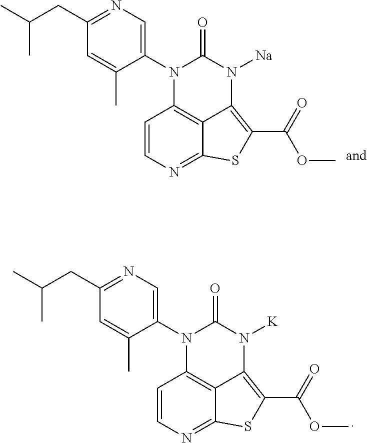

Aspect 138. A method of making tert-butyl ((1R,2S)-2-(3-chloropropanamido)cyclopentyl)carbamate

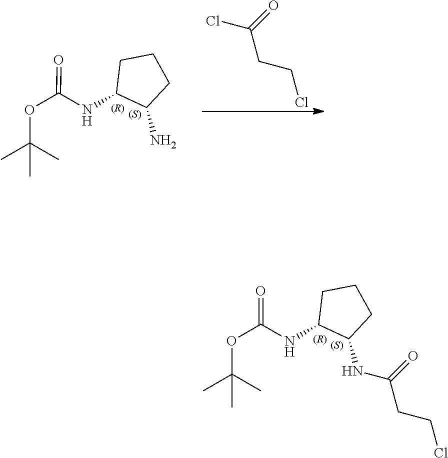

comprising contacting tert-butyl ((1R,2S)-2-aminocyclopentyl)carbamate with 3-chloropropionyl chloride in the presence of a solvent and a base.
Aspect 139. The method of aspect 138 where the solvent is methyltetrahdrofuran,
Aspect 140. The method of aspect 138 or 139 where the base is sodium hydrogen carbonate.
Aspect 141. A method of making (E)-2-cyano-3-((6-isobutyl-4-methylpyridin-3-yl)amino) but-2-enethioamide

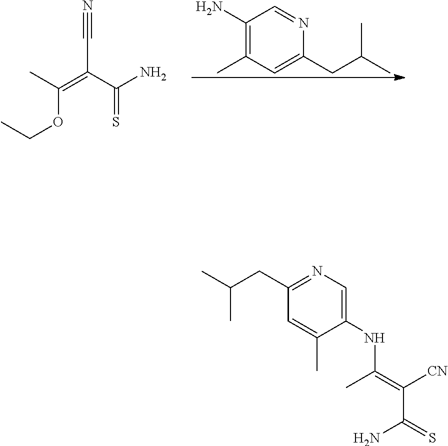 [SMILES: CCO/C(C)=C(/C#N)C(N)=S ; Cc1cc(CC(C)C)ncc1N ; C/C(Nc1cnc(CC(C)C)cc1C)=C(/C#N)C(N)=S]

comprising contacting 2-cyano-3-ethoxybut-2-enethioamide with 6-isobutyl-4-methylpyridin-3-amine in the presence of a solvent.
Aspect 142. The method of aspect 141 where the solvent is tetrahydrofuran.
Aspect 143. A method of making 4-((6-isobutyl-4-methylpyridin-3-yl)amino)-2-mercaptonicotinonitrile

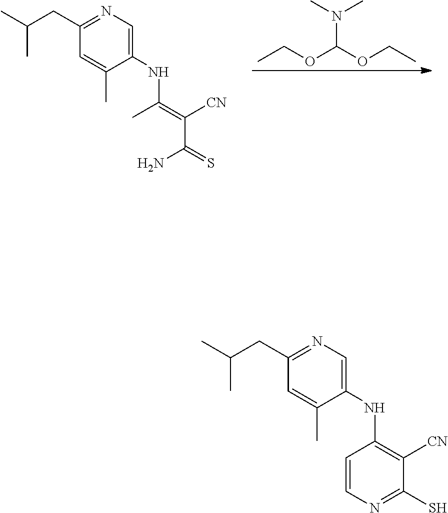

comprising contacting 2-cyano-3-((6-isobutyl-4-methylpyridin-3-yl)amino) but-2-enethioamide with N,N-dimethylformamide diethyl acetal in a solvent, in the presence of a base, followed by the addition of an acid.
Aspect 144. The method of aspect 143 where the solvent is 2-methyltetrahydrofuran.
Aspect 145. The method of aspect 143 or 144 where the base is K₂CO₃.
Aspect 146. The method of aspect 143, 144, or 145, where the acid is acetic acid.
Aspect 147. A method of making tert-butyl (6-isobutyl-4-methylpyridin-3-yl)carbamate

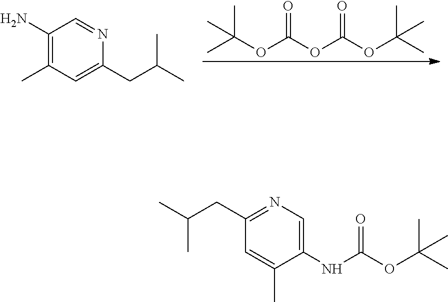

comprising contacting 6-isobutyl-4-methylpyridin-3-amine with di-tert-butyl decarbonate in the presence of a solvent system and a salt.
Aspect 148. The method of aspect 147 where the solvent system is toluene and water
Aspect 149. The method of aspect 147 or 148 where the salt is ammonium chloride.
Aspect 150. A method of making ethyl (6-isobutyl-4-methylpyridin-3-yl)carbamate

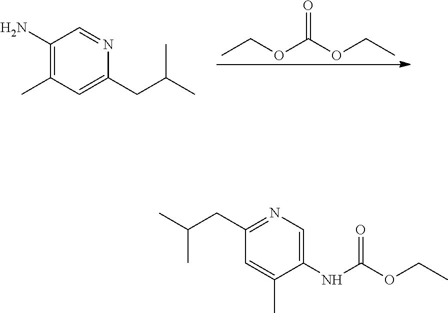

comprising contacting 6-isobutyl-4-methylpyridin-3-amine with diethyl carbonate in the presence of a solvent and a base.
Aspect 151. The method of aspect 150 where the solvent is tetrahydrofuran.
Aspect 152. The method of aspect 150 or 151 where the base is potassium tert-butoxide.
Aspect 153. A method of making methyl (6-isobutyl-4-methylpyridin-3-yl)carbamate

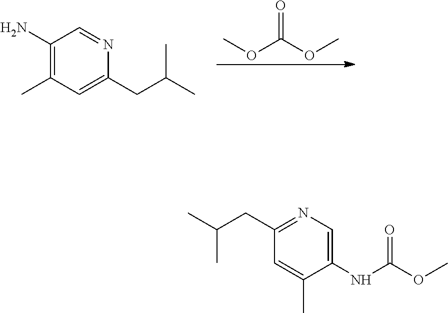

comprising contacting 6-isobutyl-4-methylpyridin-3-amine with dimethyl carbonate in the presence of a solvent and a base.
Aspect 154. The method of aspect 153 where the solvent is tetrahydrofuran.
Aspect 155. The method of aspects 153 or 154 where the base is potassium tert-butoxide.
Aspect 156. A method of making sodium 5-(6-isobutyl-4-methylpyridin-3-yl)-2-(methoxycarbonyl)-4-oxo-4,5-dihydro-1-thia-3,5,8-triazaacenaphthylen-3-ide

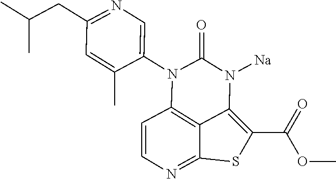

comprising the steps of
a) creating a mixture of tert-butyl (6-isobutyl-4-methylpyridin-3-yl)carbamate, with 2,4-dichloro-3-iodopyridine, and tetrabutylammonium bromide in toluene;
b) creating a second reaction mixture by adding water and sodium hydroxide to the mixture created in step a, followed by stirring the second reaction mixture for a time interval between 10 minutes and 10 hours;
c) at the conclusion of the time interval from step b, creating a third reaction mixture by adding water and methyl thioglycolate to the second reaction mixture, followed by stirring for a time interval between 10 minutes and 10 hours;
d) at the conclusion of the time interval from step c, partitioning the reaction mixture into aqueous and organic layers;
e) creating a fourth reaction mixture by adding methanol to the organic layer from step d;
f) creating a fifth reaction mixture by adding sodium hydroxide to the fourth reaction mixture, followed by heating said fifth reaction mixture to a temperature between 40° C. and 70° C. for a time interval between 10 minutes and 10 hours;
g) at the conclusion of the time interval from step f, cooling the fifth reaction mixture to a temperature between 10° C. and 30° C., followed by filtration to isolate sodium 5-(6-isobutyl-4-methylpyridin-3-yl)-2-(methoxycarbonyl)-4-oxo-4,5-dihydro-1-thia-3,5,8-triazaacenaphthylen -3-ide.
   Aspect 157. A method of making potassium 5-(6-isobutyl-4-methylpyridin-3-yl)-2-(methoxycarbonyl)-4-oxo-4,5-dihydro-1-thia-3,5,8-triazaacenaphthylen-3-ide

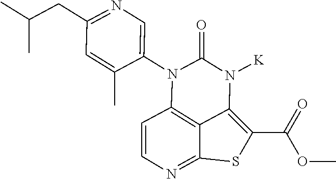

comprising the steps of
a) creating a mixture of tert-butyl (6-isobutyl-4-methylpyridin-3-yl)carbamate, with 2,4-dichloro-3-iodopyridine, and tetrabutylammonium bromide in toluene;
b) creating a second reaction mixture by adding water and sodium hydroxide to the mixture created in step a, followed by stirring the second reaction mixture for a time interval between 10 minutes and 10 hours;
c) at the conclusion of the time interval from step b, creating a third reaction mixture by adding water and methyl thioglycolate to the second reaction mixture, followed by stirring for a time interval between 10 minutes and 10 hours;
d) at the conclusion of the time interval from step c, partitioning the reaction mixture into aqueous and organic layers;
e) creating a fourth reaction mixture by adding tetrahydrofuran to the organic layer from step d;
f) creating a fifth reaction mixture by adding potassium tert-butoxide to the fourth reaction mixture, for a time interval between 10 minutes and 10 hours;
g) at the conclusion of the time interval from step f, cooling the fifth reaction mixture to a temperature between −5° C. and 15° C., followed by filtration to isolate potasium 5-(6-isobutyl-4-methylpyridin-3-yl)-2-(methoxycarbonyl)-4-oxo-4,5-dihydro-1-thia-3,5,8--triazaacenaphthylen -3-ide.

1-53. (canceled)

54. A method of synthesizing a compound of Formula (I): 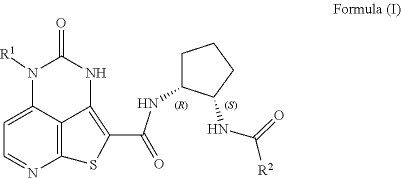
wherein:
R¹ is selected from C₅₋₁₀ aryl and C₄₋₉ heteroaryl wherein the aryl or heteroaryl are optionally substituted with one or more groups independently selected from C₁₋₆ alkyl; and
R² is selected from C₁₋₆ alkyl and C₂₋₆ alkenyl;
comprising contacting the compound 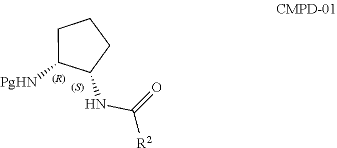
with an acid and a first solvent followed by contacting with the compound 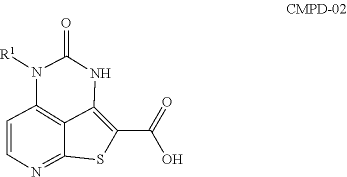
in the presence of one or more coupling reagents, an amine base, and a second solvent to obtain the compound of Formula (I).

55. The method of claim 54, wherein R¹ is

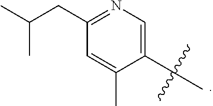 [SMILES: *c1cnc(CC(C)C)cc1C]

56. The method of claim 54, wherein R² is C₂₋₆ alkenyl.

57. The method of claim 54, wherein the acid is methanesulphonic acid.

58. The method of claim 54, wherein the coupling reagent is selected from 1-Ethyl-3-(3-dimethylaminopropyl) carbodiimide (EDCI), hydroxybenzotriazole (HOBt), 1-[Bis(dimethylamino)methylene]-1H-1,2,3-triazolo[4,5-b]pyridinium 3-oxide hexafluorophosphate (HATU), propylphosphonic anhydride (T3P), 1,1′-carbonyldiimidazole (CDI), 2-hydroxypyridine-N-oxide (HOPO), and combinations thereof.

59. The method of claim 54, wherein the amine base is selected from N,N-diisopropylethylamine (DIPEA), triethylamine, tributylamine, N-methyl morpholine, and N-methyl piperidine. In some aspects the second solvent is selected from dichloromethane, ethyl acetate, 2-methyltetrahydrofuran, tetrahydrofuran, and acetonitrile.

60. The method of claim 54, further comprising contacting the compound 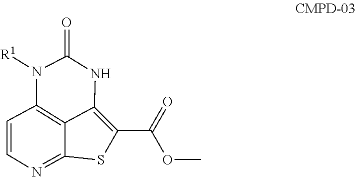
with a base and an ammonium salt in a solvent to form the compound

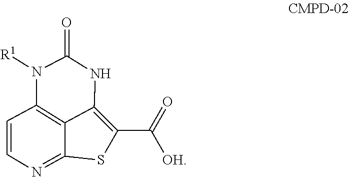

61. The method of claim 60, further comprising contacting the compound 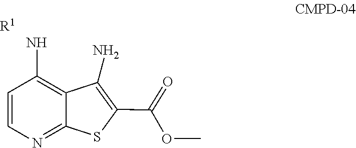
with a carbonyl source and a solvent to form the compound

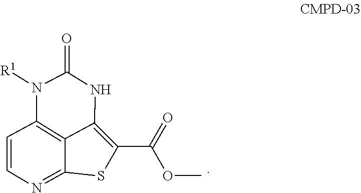

62. The method of claim 61, further comprising contacting the compound 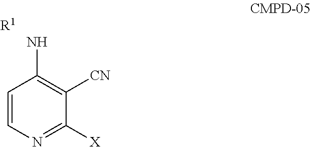
with a sulfur reagent and a base in a solvent to form the compound 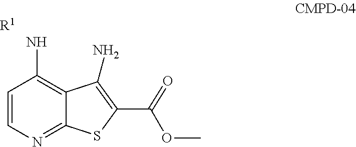
and wherein X is selected from Cl and Br.

63. The method of claim 62, further comprising contacting the compound 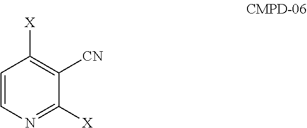
with the compound R¹—NH₂ with a base, and an amine base in a solvent system to form the compound

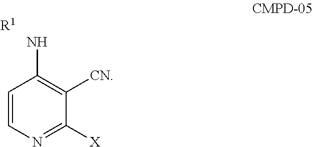

64. The method of claim 63, further comprising contacting the compound 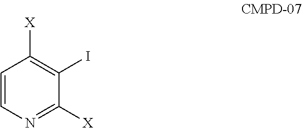
with a cyano source and a solvent to form the compound

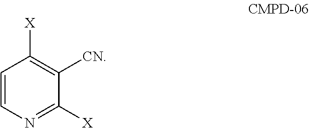

65. The method of claim 64, further comprising contacting the compound 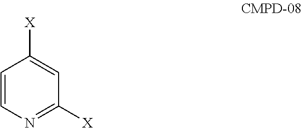
with a base and an electrophilic halogen in a solvent to form the compound

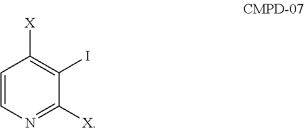

66. The method of claim 54, further comprising contacting the compound 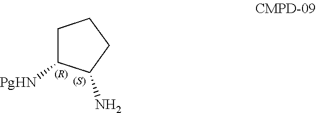
with a base, 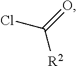
and a solvent to form the compound

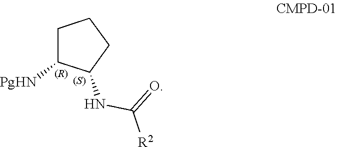

67. A method of synthesizing a compound of Formula (II): 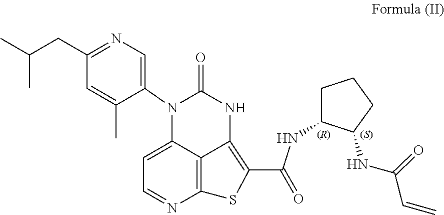
comprising contacting the compound 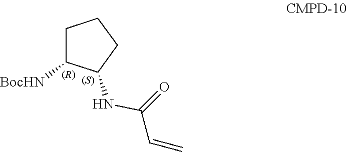
with an acid and a first solvent followed by contacting with the compound 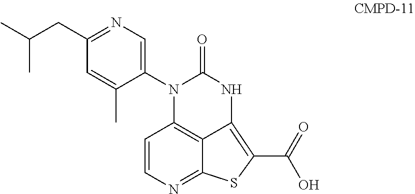 [SMILES: Cc1cc(CC(C)C)ncc1N1C(=O)Nc2c(C(=O)O)sc3nccc1c23]
in the presence of one or more coupling reagents, an amine base, and a second solvent to obtain the compound of Formula (II).

68. The method of claim 67, further comprising contacting the compound 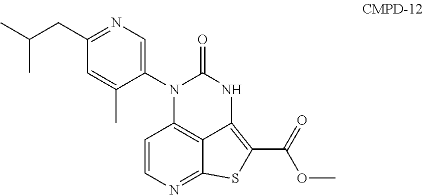 [SMILES: COC(=O)c1sc2nccc3c2c1NC(=O)N3c1cnc(CC(C)C)cc1C]
with a base and an ammonium salt in a solvent to form the compound

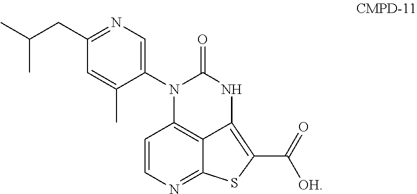 [SMILES: Cc1cc(CC(C)C)ncc1N1C(=O)Nc2c(C(=O)O)sc3nccc1c23]

69. The method of claim 68, further comprising contacting the compound 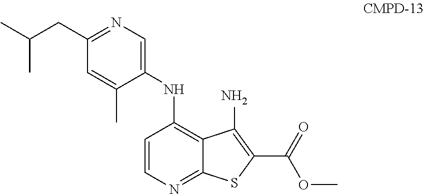 [SMILES: COC(=O)c1sc2nccc(Nc3cnc(CC(C)C)cc3C)c2c1N]
with a carbonyl source and a solvent to form the compound

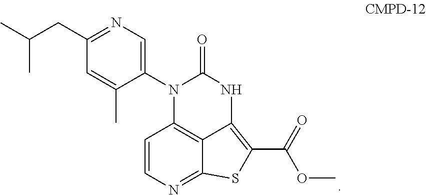 [SMILES: COC(=O)c1sc2nccc3c2c1NC(=O)N3c1cnc(CC(C)C)cc1C]

70. The method of claim 69, further comprising contacting the compound 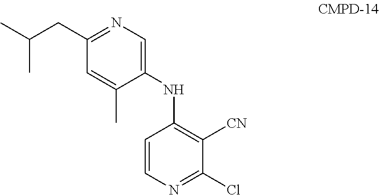 [SMILES: Cc1cc(CC(C)C)ncc1Nc1ccnc(Cl)c1C#N]
with a sulfur reagent and a base in a solvent to form the compound

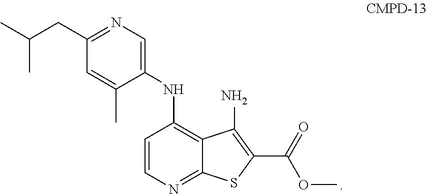 [SMILES: COC(=O)c1sc2nccc(Nc3cnc(CC(C)C)cc3C)c2c1N]

71. The method of claim 70, further comprising contacting the compound 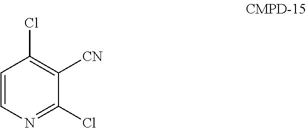
with the compound 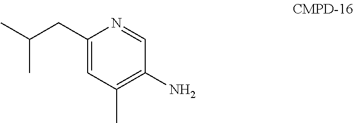
with a base, and an amine base in a solvent system to form the compound

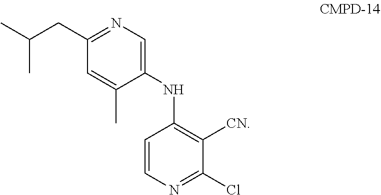 [SMILES: Cc1cc(CC(C)C)ncc1Nc1ccnc(Cl)c1C#N]

72. The method of claim 71, further comprising contacting the compound 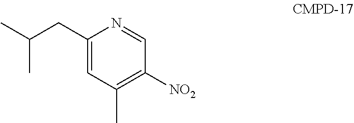
with a hydride source, a catalyst, and a solvent system to form the compound

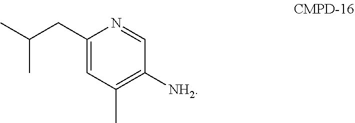

73. The method of claim 72, further comprising contacting the compound 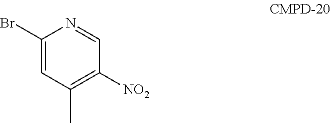
with a catalyst, phosphorus reagent, a boron reagent, a first base, a second base and a solvent system to form the compound

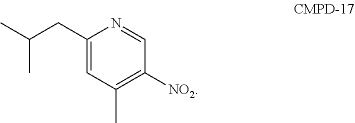

74. The method of claim 73, further comprising contacting the compound 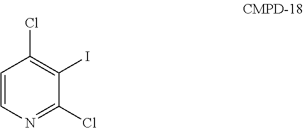
with a cyano source and a solvent to form the compound

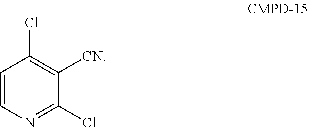

75. The method of claim 74, further comprising contacting the compound 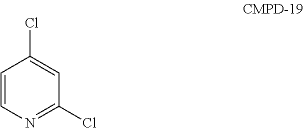
with a base and an electrophilic halogen in a solvent to form the compound

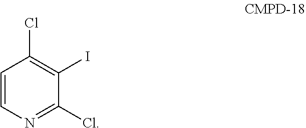

76. The method of claim 75, further comprising contacting the compound 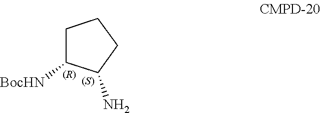
with a base, 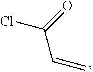
and a solvent to form the compound

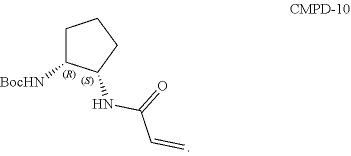

77. A method of isolating the P atropisomer from a mixture of M and P isomers of compound of Formula (II), comprising the steps of:
1) Dissolving the compound of Formula (II) in one or more solvents in a vessel to form a solution;
2) Adding water to the solution and waiting for a time period of about 2 hours to 24 hours to form a suspension;
3) Filtering the suspension to obtain the crystals of compound of Formula P-(II);
4) Collecting the filtrate and heating the filtrate to a temperature of about 75° C. to about 150° C. for a time period of about about 5 minutes to about 1 hour;
5) Cooling the filtrate to a temperature of about 20° C. to about 30° C.;
6) Repeating steps 3-5 about 20-100 times until the M isomer is converted to the P isomer,
7) Collecting the precipitate, washing the precipitate, and drying the precipitate under humidified conditions to obtain the compound of Formula P-(II).

78. A crystalline Form B of Formula P-(II) dihydrate:

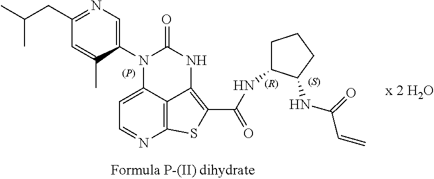

79. The crystalline Form B of claim 78, wherein crystalline Form B is characterized by a XRPD pattern having a peak expressed in degrees 2θ (±0.2) at about 5.599°, and further characterized by a XRPD pattern having peaks expressed in degrees 2θ (±0.2) at about 20.426°, about 24.665°, about 11.135°, about 26.373°, about 12.134°, about 23.187°, about 19.065°, and about 30.316°.
